(19) United States
(12) Patent Application Publication
(10) Pub. No.: US 2008/0311159 A1
(43) Pub. Date: Dec. 18, 2008

(54) Methods and Means for Diagnostics, Prevention and Treatment of Mycobacterium Infections and Tuberculosis Disease

(76) Inventors: Michel Robert Klein, Zaandijk (NL); Min Yong Lin, Leiden (NL); Krista Elisabeth van Meijgaarden, Delft (NL); Cornelus Leonardus Maria Coleta Franken, Leiden (NL); Eliane Madeleine Sophie Leyten, Den Haag (NL); Tom Henricus Maria Ottenhof, Leiden (NL)

Correspondence Address: MORGAN LEWIS & BOCKIUS LLP, 1111 PENNSYLVANIA AVENUE NW, WASHINGTON, DC, 20004, US

(21) Appl. No.: 11/910,125

(22) PCT Filed: Mar. 31, 2006
(86) PCT No.: PCT/NL2006/050068

§ 371 Date: May 13, 2008

(30) Foreign Application Priority Data
Mar. 31, 2005 (EP) 05075748.3

(51) Int. Cl. A61K 39/00; C12Q 1/02; A61P 37/00

(52) U.S. Cl. 424/248.1; 435/29

(57) ABSTRACT

The invention identifies a narrow subset of Mycobacterium latency associated antigens and or epitopes that are capable of eliciting an immune response in vivo and in vitro in a mammal. The invention provides methods and compositions for detection and immunization against latent Mycobacterium infections. These compositions comprise those Mycobacterial latency antigens that are actually capable of eliciting an immune response in vivo, in mammals experiencing a latent Mycobacterium infection. More preferably the compositions comprise those antigens that are preferentially recognized by latently infected individuals and which antigens are not, or to a much lesser extent, recognized in individuals having an active Mycobacterium infection or in individuals having Mycobacterium induced symptoms or diseases, such as in patients infected with M. tuberculosis suffering from tuberculosis disease (TB).

### FIELD OF THE INVENTION

The current invention relates to the field of medicine, in particular to diagnosis, prevention and treatment of Mycobacterial diseases, more in particular to those infections caused by Mycobacterium tuberculosis. The invention also relates to the field of vaccination.

### BACKGROUND OF THE INVENTION

Tuberculosis (TB) is a major threat to global health, with a conservative estimate of four persons dying of TB every minute, corresponding to two million yearly. It has been estimated that one third of the world population is latently infected with M. tuberculosis. This enormous reservoir of latent tuberculosis, from which most cases of active TB arise, embodies a major obstacle in achieving control of TB.

There are several reasons why latent M. tuberculosis infections complicate the efforts to eliminate TB. Firstly, contact tracing and treatment of latent infection is only achievable in a setting where most persons are tuberculin skin test negative, this being the case in industrialized countries where TB incidence is already low. Even in that setting, the effectiveness of the currently available regimens used for the treatment of latent M. tuberculosis infection is limited. Apart from obvious problems with low treatment adherence and prevalence of antibiotic resistant strains, this could be related to the finding that dormant M. tuberculosis organisms are moderately to highly resistant to commonly used drugs such as rifampin and isoniazid that are bactericidal only to replicating bacilli, as has been demonstrated in vitro (1,2). The idea of M. tuberculosis non-replicating persistence during latency is supported by the finding that the genotype of M. tuberculosis hardly changes during many years of latency, while the rate of changes in DNA patterns is much higher during active disease (3,4). Secondly, the only currently available vaccine against TB, M. bovis bacillus Calmette-Guérin (BCG), does not prevent the establishment of latent M. tuberculosis infection. BCG provides a highly variable level of protection against reactivation TB that differs between geographic regions and which is probably dependent on the level of exposure to environmental mycobacteria (5). Recent efforts towards the development of an improved vaccine have mainly focused on prophylactic vaccines that are intended to be administered before infection with M. tuberculosis has occurred, and that have been evaluated in animal models of acute primary infection. These prophylactic vaccine candidates were ineffective or even deleterious when used in a post-exposure setting using animal models mimicking either chronic or latent infection (6-8). In contrast, a post-exposure vaccine that can be safely administered to already latently infected individuals and that prevents reactivation of TB will have the immediate advantage that it can be applied in high endemic areas where latent infection with M. tuberculosis is present in the majority of the population. The antigens to be included in such a vaccine should enhance a protective immune response able to recognize and eliminate M. tuberculosis bacilli during latent infection.

CD4 T cells play an important role in controlling and maintaining M. tuberculosis infection, yet the precise mechanisms involved and the target antigens recognized during latent TB are largely unknown (9). Until recently, few studies have addressed differential gene expression and changes in metabolism of M. tuberculosis during latency. Even fewer studies have analyzed specific human host immune responses that are associated with maintenance of latency. Up to now only one well characterized M. tuberculosis protein is identified which seems to be of importance during latency (10). This protein, HspX (Rv2031c or Acr), is strongly upregulated during hypoxia, an in vitro condition used as a proxy of the environmental stress associated with latent infection in human granulomas (11). Cellular immune responses to this heat-shock protein were observed in latently infected individuals, thus in association with a protected state, while antibodies to this antigen were found in persons with active TB disease (12, 13).

Identification of additional latency associated antigens is beneficial for the development of successful post-exposure vaccines. Several studies have focused on the persistent state of M. tuberculosis infection using in vitro and in vivo models that were developed to mimic the natural state of latency. First, Wayne et al. established an in vitro model of latency by growing M. tuberculosis under gradually decreasing oxygen tensions which resulted in a reversible growth arrest of the bacilli, named non-replicating persistence (NRP) (14). Others used constant hypoxic culture conditions to study the metabolic changes of M. tuberculosis (11,15,16). In addition, Voskuil et al. studied expression profiles of M. tuberculosis, when cultured in the presence of low dose nitric oxide, as yet another in vitro condition encountered by bacilli during latency and coincide with the (onset) of Th1 immunity (19). Using whole genome DNA microarrays, Voskuil et al. observed that a set of 48 genes of M. tuberculosis was upregulated consistently, observed in all three in vitro models of latency, namely during NRP, constant hypoxia and during low dose nitric oxide exposure (17). Genes of this so-called dormancy regulon (DosR) were also found to be upregulated when M. tuberculosis was grown in activated murine macrophages in vitro (18). Also in the lungs of chronically infected mice and of TB patients, the transcription pattern of M. tuberculosis showed characteristics of NRP (19, 20). This suggests that during the course of TB disease most likely a subpopulation of bacilli encounters hypoxic conditions and low concentrations of nitric oxide, and is adapting to a nonreplicating state. It appears that dormancy-associated proteins are induced during the transition process to latent infection and that during latency most likely tubercle bacilli will be expressing dormancy-associated proteins. The function of most putative proteins encoded by this regulon is unknown. In this patent specification, we will refer to the proteins encoded by genes of the dormancy regulon as latency antigens.

Based upon the discovery of the dormancy (DosR) regulon genes, use of the encoded proteins as potential antigens for detection purposes and protective or curative immunization and/or vaccination purposes was envisaged in GB 0116385.6/US2004/0241826. Furthermore, WO 0179274 and US2004/0057963 provide methods and compositions aimed at inducing an immune response to latent Mycobacterium tuberculosis infections, using polypeptides which are induced specifically during the latent stage of mycobacterial infections. The polypeptides therein are selected from a pool of 45 dormancy regulon genes, which are upregulated during latency in the aforementioned in vitro models. One of the antigens that has been put forward for immunization purposes is HspX (Rv2031c or Acr), encoding an alpha crystallin homolog, and is described to be useful for diagnostic and immunization purposes in US2004/0146933.

It is currently not known whether NRP/dormancy (DosR) regulon encoded proteins are actually expressed at sufficiently high levels by M. tuberculosis during the latency phase of infections in humans in order to induce an significant immune response, because the prior art used in vitro models and mouse models for latency. Also it is not known whether the putative antigens from the latency or dormancy regulon are sufficiently immunogenic and whether immunity to these hypothetical latency antigens and/or epitopes is indeed relevant for providing protection against latent or newly acquired Mycobacterium infections in mammals. In particular, it is currently not known which of the 48 dormancy/latency regulon encoded putative antigens might be most relevant during actual human Mycobacterium tuberculosis latent infections.

It is not feasible or desirable, nor effective to combine, for instance in a pharmaceutical composition or a vaccine, all 48 putative latency antigens in order to elicit an immune response in an individual having a latent Mycobacterium infection. Many of the identified putative latency antigens will not be effective in eliciting an immune response, because they are not expressed at sufficient levels or because they do not contain sufficient (dominant) cytotoxic T cell (CTL) or T helper (Th) epitopes that are recognizable for the immune system of the latently infected mammal.

### SUMMARY OF THE INVENTION

The problem to be solved by the invention is to provide an optimal choice from the 48 known putative latency antigens and to select only those antigens that are actually capable of eliciting in vivo immune responses in healthy individuals with latent Mycobacterium infection. The current invention addressed the problems discussed above by the ex vivo identification of dominant human immune responses against Mycobacterium latency associated antigens and/or epitopes in vivo and thereby provides new methods and compositions for detection and immunization against latent Mycobacterium infections. These compositions comprise only those latency antigens that actually are capable of eliciting an immune response in vivo, in mammals experiencing a latent Mycobacterium infection, and more preferably comprises only those antigens that are preferentially recognized by latently infected individuals and that are not, or to a much lesser extent, recognized in individuals having an active Mycobacterium infection or in individuals having Mycobacterium induced diseases or symptoms, such as in patients suffering from tuberculosis (TB). The invention achieves this goal by identifying a narrow subset of dominant antigens and/or epitopes from the group of at least 48 M. tuberculosis latency antigens known in the art.

Surprisingly, the most preferred antigens identified in the current invention differ from those putative latency antigens that have been most studied and applied so far in the prior art; mainly Rv2031c (HspX/acr) and Rv0569. The current invention teaches away from the preferred putative latency antigens selected and applied in prior art publications, patents and patent applications, demonstrating that the narrow subset of the current invention is far removed from known examples. The distinct and small subset of latency antigens according to this disclosure is a purposive selection from the known group of putative latency antigens. The antigens and/or epitopes according to this invention have been selected after extensive analysis of the group of putative antigens. Whereas putative antigens in the prior art were identified in in vitro models under laboratory conditions and in mouse models, the current invention discloses which of those antigens are actually capable of differentially eliciting an immune response in vivo under normal circumstances by latent Mycobacterium infections, and not or to a lesser extent in healthy individuals or patients suffering from TB disease.

### DETAILED DESCRIPTION OF THE INVENTION

The current invention provides methods and compositions for inducing an immune response to Mycobacterium infections in a vertebrate, preferably a mammal, in particular to latent Mycobacterium infections, the method comprising the step of administering to the vertebrate a composition comprising a source of one or more polypeptides or fragments thereof selected from the group of polypeptides comprising Mycobacterium NRP/dormancy (DosR) regulon encoded proteins that are capable of eliciting an immune response in vivo in vertebrates having a Mycobacterium infection.

The term Mycobacterium infection herein is meant to comprise both latently infected mammals, newly infected mammals not yet exhibiting symptoms and vertebrates suffering from Mycobacterium induced disease and symptoms, such as in active tuberculosis. Preferably, the Mycobacterium infection to be treated according to the invention is a latent infection in order to prevent development of tuberculous disease. The method according to the invention may also be advantageously applied as adjunctive therapy during or following antibiotic treatment of TB patients, with or without (multiple) drug-resistant TB; and to healthy but exposed persons, preferably but not exclusively children from TB endemic countries who have previously been vaccinated with BCG.

The invention provides methods and compositions which may be aimed at latent Mycobacterium infections, but may also easily be combined by the skilled person with polypeptides or compositions comprising epitopes aimed at eliciting an immune response to non-latent infections, such as prophylactic vaccines and/or multiphase vaccines against Mycobacteria.

A latent Mycobacterial infection is herein understood to refer to a stage in the infection where the bacilli remain viable but are slowly replicating or persisting in a non-replicating state and may be encapsulated in localized lesions within an organ or tissue, not causing active necrotic disease, as typically observed in TB. The latent stage may exist for the remainder of a host's life, or the infection may reactivate during, for instance, a period of decreased host immunity or in response to other stressors, such as other (myco)bacterial or viral infections like HIV-1 or treatment for cancer and other immune suppressive conditions or treatments.

The invention provides methods and compositions which are suitable for use as 1) preventive (prophylactic), 2) post-exposure/infection or 3) therapeutic/curative vaccines against latent Mycobacterium infections and related diseases, such as, but not limited to (live-attenuated and/or recombinant) Mycobacterium tuberculosis, M. bovis (including Bacillus Calmette-Guerin (BCG), M. africanum, M. smegmatis, M. leprae, M. vaccae, M. intracellulare, M. avium (including subsp. paratuberculosis), M. canettii, M. leprae, M. microti and M. ulcerans.

M. tuberculosis, M. bovis (including BCG strains), M. microti, M. africanum and M. canettii (i.e. Mycobacterium species and strains belonging to the TB complex) are the most preferred sources of latency induced polypeptides or fragments thereof to be used according to the invention.

For the methods and compostions of the invention, the vertebrate to be treated or diagnosed preferably is a human, but also comprises all laboratory and farm animals, such as but not limited to, mice, rats, guinea pigs, rabbits, cats, dogs, sheep, goats, cows, horses, camels and poultry like e.g. chicken, ducks, turkey and geese.

The source of the polypeptide may be a protein, a digest of the protein and/or fragments thereof, which may be in a purified form or may be comprised within a crude composition, preferably of biological origin, such as bacterial lysates, sonicates or fixates. Alternatively, the (poly)peptide may be chemically synthesized or enzymatically produced in vitro. The source of the polypeptide or fragment thereof may also be a nucleic acid encoding the polypeptide or fragment thereof, from a RNA or DNA template. The RNA or DNA molecules may be ‘naked’ DNA, preferably comprised in vesicles or liposomes, or may be comprised in a vector. The vector may be any (recombinant) DNA or RNA vector known in the art, and preferably is a plasmid wherein genes encoding latency antigens are operably linked to regulatory sequences conferring expression and translation of the encoded messengers. The vector may also be any DNA or RNA virus, such as but not limited to Adenovirus, Adeno-Associated Virus (AAV), a retrovirus, a lentivirus, modified Vaccinia Ankara virus (MVA) or Fowl Pox virus, or any other viral vector capable of conferring expression of polypeptides comprising latency epitopes to a host. DNA vectors may be non-integrating, such as episomally replicating vectors or may be vectors integrating in the host genome by random integration or by homologous recombination.

DNA molecules comprising genes encoding the polypeptides or fragments thereof according to the current invention, optionally embedded in vectors such as viruses or plasmids, may be integrated in a genome of a host. In a preferred embodiment of the invention, such a host may be a micro-organism. Preferably such a recombinant micro-organism is a Mycobacterium, for instance of the species M. tuberculosis or M. bovis and most preferably M. bovis Bacillus Calmette Guerin (BCG), capable of delivering to a host the polypeptides or fragments thereof according to the invention. Recombinant BCG and methods for recombination are known in the art, for instance in WO2004094469. Such a recombinant micro-organism may be formulated as a live recombinant and/or live attenuated vaccine, as for instance in Jacobs et al. 1987, Nature, 327(6122):532-5). The vector may also be comprised in a host of bacterial origin, such as but not limited to live-attenuated and/or recombinant Shigella or Salmonella bacteria.

In one embodiment, the current invention provides a method for the induction of an immune response to a Mycobacterium infection in a mammal, the method comprising the step of administering to the mammal a source of one or more polypeptides or fragments thereof selected from the group of polypeptides comprising Mycobacterium NRP/dormancy (DosR) regulon encoded proteins that are capable of eliciting an IFN-γ response in human T cell lines, consisting of Rv079, Rv0569, Rv0572c, Rv1733c, Rv1738, Rv1813c, Rv1996, Rv2007c (FdxA), Rv2029c (PfkB), Rv2030c, Rv2031c (HspX, Acr, 16-kDa alpha crystallin homolog), Rv2032, Rv2623, Rv2624c, Rv2626c, Rv2627c, Rv2628, Rv3126c, Rv3127, Rv3129, Rv3130c, Rv3131, Rv3132c, Rv3133c (DosR), Rv3134c, Rv0080, Rv1737c (NarK2), Rv1735c and Rv1736c (NarX) and analogues or homologues thereof, and optionally one or more adjuvants.

Said antigens are recognizable to short-term T-cell lines generated from infected individuals and brought into contact with a M. tuberculosis sonicate. The T cell lines exhibit an interferon gamma (IFN-γ) response of preferably at least >50 pg IFNγ/ml in an assay as described in examples 1 and 2. The Rv nomenclature for Mycobacterial antigens and the DNA and protein sequences of the NRP/dormancy (DosR) regulon are well known in the art and may for instance be found at: http://genolist.pasteur.fr/TubercuList/ or or at http://www.ncbi.nlm.nih.gov/entrez (Accession number AL123456). The Rv nomenclature as used herein may refer to either the amino acid sequence of the antigen or the nucleotide sequence encoding the antigen.

In a preferred embodiment of the invention, the method for the induction of an immune response to a Mycobacterium infection in a vertebrate comprises the administration of a source of polypeptides or fragments thereof which are selected from the group of Mycobacterium NRP/dormancy (DosR) regulon sequences that contain latency antigens capable of eliciting an immune response in vertebrates having a latent Mycobacterium infection, consisting of Rv0079, Rv0569, Rv1733c, Rv1738, Rv1813c, Rv1996, Rv2007c (FdxA), Rv2029c (PfkB), Rv2030c, Rv2031c (HspX, Acr, 16-kDa alpha crystallin homolog), Rv2032, Rv2626c, Rv2627c, Rv2628, Rv3126c, Rv3129, Rv3130c, Rv3132c, Rv3133c (DosR), Rv0080, Rv1737c (NarK2), Rv1735c and Rv1736c (NarX) and homologues or analogues thereof. This particular subset of latency antigens is capable of inducing an interferon γ (IFN γ) response in peripheral blood monocytes (PBMC's) from individuals having a latent Mycobacterial infection of more than 100>pg IFNγ/ml and in at least 5, 10, 20, 30, 40 or 50% of all Mycobacterium infected individuals.

In a most preferred embodiment of the invention, there is provided a method of inducing an immune response in a vertebrate, preferably in an individual suffering from or at risk of acquiring a latent Mycobacterium infection, comprising the administration of a source of polypeptides or fragments thereof which are selected from the group of Mycobacterium NRP/dormancy (DosR) regulon sequences that are capable of preferentially eliciting an immune response in individuals having a latent Mycobacterium infection, consisting of antigens Rv1733c, Rv2029c (PfkB), Rv2627c, Rv2628, Rv0080, Rv1737c (NarK2), Rv1735c and Rv1736c (NarX). Said eight antigens comprise dominant epitopes which are preferentially recognized in latently infected individuals and which are not, or to a much lesser extent, capable of inducing an IFN-γ response in non infected individuals or in individuals having an active Mycobacterium infection causing disease symptoms. Said antigens induced the highest levels of IFN-γ in peripheral blood mononuclear cells (PBMC's) from the 48 latency antigens tested. In addition, these eight antigens are capable of inducing a significant IL-10 production in PBMC's from latently infected individuals, but not in PBMC's from patients suffering from symptoms associated with active Mycobacterium tuberculosis infections.

The three most preferred polypeptides to be used in the method according to this invention are the Mycobacterium NRP/dormancy (DosR) regulon sequence Rv1733c and Rv2029c (PfkB) and Rv2627c. Of all 48 polypeptides tested Rv1733c and Rv2029c (PfkB) and Rv0080 are the most frequently detected to elicit immune responses in individuals with latent Mycobacterium infection, as determined by induction of IFN-γ and/or IL-10 in PBMC's obtained from these individuals.

In another aspect of the invention, the invention provides compositions comprising a source of polypeptides or fragments thereof of Mycobacterium NRP/dormancy (DosR) regulon sequences that are capable of eliciting an immune response in individuals having a latent Mycobacterium infection. Preferably the composition for immunization against Mycobacterium infections and induced diseases according to the invention comprises a source of one or more polypeptides or fragments thereof selected from the group of polypeptides comprising Mycobacterium NRP/(DosR) regulon encoded proteins capable of eliciting an IFNγ response in human T-cell lines, consisting of Rv0079, Rv0569, Rv0572c, Rv1733c, Rv1738, Rv1813c, Rv1996, Rv2007c (FdxA), Rv2029c (PfkB), Rv2030c, Rv2031c (HspX, Acr, 16-kDa alpha crystallin homolog), Rv2032, Rv2623, Rv2624c, Rv2626c, Rv2627c, Rv2628, Rv3126c, Rv3127, Rv3129, Rv3130c, Rv3131, Rv3132c, Rv3133c (DosR), Rv3134c, Rv0080, Rv1737c (NarK2), Rv1735c and Rv1736c (NarX) and analogues or homologues thereof, and optionally comprising at least one adjuvant.

A homologue or analogue herein is understood to comprise a peptide having at least 70% 80, 90, 95, 98 or 99% amino acid sequence identity with the native M. tuberculosis NRP/dormancy (DosR) regulon encoded polypeptides mentioned above and is still capable of eliciting at least the immune response obtainable by the M. tuberculosis polypeptide. A homologue or analogue may comprise substitutions, insertions, deletions and additional N- or C-terminal amino acids or chemical moieties to increase stability, solubility and immunogenicity.

A fragment of the polypeptide antigens of the invention is understood to be a fragment comprising at least an epitope. The fragment therefore at least comprises 4, 5, 6, 7 or 8 contiguous amino acids from the sequence of the polypeptide antigen. More preferably the fragment comprises at least a T cell epitope, i.e. at least 8, 9, 10, 11, 12, 13, or 14 contiguous amino acids from the sequence of the polypeptide antigen. Still more preferably the fragment comprises both a CTL and a T helper epitope. Most preferably however, the fragment is a peptide that requires processing by an antigen presenting cell, i.e. the fragment has a length of at least about 18 amino acids, which 18 amino acids are not necessarily a contiguous sequence from the polypeptide antigen.

More preferably, the composition of the invention comprises a source of polypeptides or fragments thereof which are selected from the group of Mycobacterium NRP/dormancy (DosR) regulon sequences comprising latency antigens capable of eliciting an immune response in Mycobacterium latently infected individuals, consisting of Rv0079, Rv0569, Rv1733c, Rv1738, Rv1813c, Rv1996, Rv2007c (FdxA), Rv2029c (PfkB), Rv2030c, Rv2031c (HspX, Acr, 16-kDa alpha crystallin homolog), Rv2032, Rv2626c, Rv2627c, Rv2628, Rv3126c, Rv3129, Rv3130c, Rv3132c, Rv3133c (DosR), Rv0080, Rv1737c (NarK2), Rv1735c and Rv1736c (NarX). Said antigens and compositions are capable of inducing an IFNγ response in peripheral blood mononuclear cells (PBMC's) from individuals having a Mycobacterium infection.

In a more preferred embodiment, a composition according to the invention comprises a source of Mycobacterium NRP/dormancy (DosR) regulon sequences that are capable of preferentially eliciting an immune response in individuals having a latent Mycobacterium infection, whereby the antigens are selected from one or more of: Rv1733c, Rv2029c (PfkB), Rv2627c, Rv2628, Rv0080, Rv1737c (NarK2), Rv1735c and Rv1736c (NarK). The Rv1733c, Rv2029c (PfkB), Rv2627c, Rv2628, Rv0080, Rv1737c (NarK2), Rv1735c and Rv1736c (NarX) polypeptides are capable of inducing the strongest response in individuals with latent infections in terms of IFN γ production in PBMC's in comparison to the other 48 Mycobacterium NRP/dormancy (DosR) regulon polypeptides tested. Said antigens are also capable of stimulating IL-10 production in PBMC's from latently infected patients, whereas IL-10 induction is not, or to a much lesser extent, observed in PBMC's from patients having an active Mycobacterium infection and/or TB disease symptoms.

In another embodiment, the composition according to the invention comprises at least a source of the polypeptide or fragments thereof are obtained from the Mycobacterium NRP/dormancy (DosR) regulon sequence Rv1733c and/or Rv2029c and/or Rv2627c, which are the most frequently recognized antigens in latent infected individuals from all NRP/dormancy (DosR) regulon encoded polypeptides assayed.

The composition for immunization according to the invention comprising NRP/dormancy (DosR) regulon encoded polypeptides or fragments thereof preferably comprises at least one excipient. Excipients are well known in the art of pharmacy and may for instance be found in textbooks such as Remmington's pharmaceutical sciences, Mack Publishing, 1995. The composition for immunization according to the invention may preferably comprise at least one adjuvant. Adjuvants may comprise any adjuvant known in the art of vaccination and may be selected using textbooks like Current Protocols in Immunology, Wiley Interscience, 2004.

Adjuvants are most preferably selected from the following list of adjuvants: cationic (antimicrobial) peptides and Toll-like receptor (TLR) ligands such as but not limited to: poly(I:C), CpG motifs, LPS, lipid A, lipopeptide Pam3Cys and bacterial flagellins or parts thereof, and their derivatives having chemical modifications. Other preferred adjuvants for use in the method and in compositions according to the invention are: mixtures with live or killed BCG, immunoglobulin complexes with the said latency antigens or parts thereof, IC31 (from www.intercell.com; in WO03047602), QS21/MPL (US2003095974), DDA/MPL (WO2005004911), DA/TDB (WO2005004911; Holten-Andersen et al, 2004 Infect Immun. 2004 March; 72(3):1608-17.) and soluble LAG3 (CD223) (from www.Immunotep.com; US2002192195).

The method and the composition for immunization according to the current invention may further comprise the use and/or addition of a CD40 binding molecule in order to enhance a CTL response and thereby enhance the therapeutic effects of the methods and compositions of the invention. The use of CD40 binding molecules is described in WO 99/61065, incorporated herein by reference. The CD40 binding molecule is preferably an antibody or fragment thereof or a CD40 Ligand or a variant thereof, and may be added separately or may be comprised within a composition according to the current invention.

The method and the composition for immunization according to the current invention may further comprise the use and/or addition an agonistic anti-4-1BB antibody or a fragment thereof, or another molecule capable of interacting with the 4-1BB receptor. The use of 4-1BB receptor agonistic antibodies and molecules is described in WO 03/084999, incorporated herein by reference. 4-1BB agonistic antibodies may be used with or without the addition of CD40 binding molecules, in order to enhance CTL immunity through triggering/stimulating the 4-1BB and/or CD40 receptors. The 4-1BB binding molecule or antibody may be added separately or may be comprised within a composition according to the current invention

Polypeptides according to the invention may for immunization purposes be fused with proteins such as but not limited to tetanus toxin/toxoid, diphtheria toxin/toxoid or other carrier molecules. The polypeptides according to the invention may also be advantageously fused to heatshock proteins, such as recombinant endogenous (murine) gp96 (GRP94) as a carrier for immunodominant peptides as described in (references: Rapp UK and Kaufmann S H, Int Immunol. 2004 April; 16(4):597-605; Zugel U, Infect Immun. 2001 June; 69(6):4164-7) or fusion proteins with Hsp70 (Triebel et al; WO9954464).

The method and the composition for immunization according to the invention preferably comprises the use of polypeptide fragments obtained from the polypeptides according to the invention comprising dominant CTL or Th epitopes and which peptides are between 18 and 45 amino acids in length. The presence of CTL or Th epitopes in a sequence may be found by the skilled artisan using commonly known bio-informatics tools such as HLA_BIND, SYFPEITHI, NetMHC and TEPITOPE 2000 (refs. 43, 44, 45, 46, 47 and 48) or experimentally using standard experimentation (Current Protocols in Immunology, Wiley Interscience 2004).

Peptides according to the invention having a length between 18 and 45 amino acids have been observed to provide superior immunogenic properties as is described in WO 02/070006. Peptides may advantageously be chemically synthesized and may optionally be (partially) overlapping and/or may also be ligated to other molecules, such as TLR ligands, peptides or proteins. Peptides may also be fused to form synthetic proteins, as in PCT/NL03/00929 and in Welters et al. (Vaccine. 2004 Dec. 2; 23(3):305-11). It may also be advantageous to add to the amino- or carboxy-terminus of the peptide chemical moieties or additional (modified or D-) amino acids in order to increase the stability and/or decrease the biodegradability of the peptide. To improve the immunogenicity/immuno-stimulating moieties may be attached, e.g. lipidation. To enhance the solubility of the peptide, addition of charged or polar amino acids may be used, in order to enhance solubility and increase stability in vivo.

In yet another embodiment, the composition for eliciting an immune response or immunization according to the invention further comprises Mycobacterium antigens that are not specific for the latency stage. Such antigens may advantageously be highly specific for other stages of the infectious process. It may be beneficial for immunization purposes to provide compositions which are not only solely directed at eliciting an immune response to latent Mycobacterium infections but which are also capable of eliciting an immune response directed at active Mycobacterium infections causing symptoms of disease in the infected mammal. In such methods and for such compositions it is useful to combine protective immunity against Mycobacteria at various phases of the infectious process and thereby provide a better overall protection. Compositions according to the invention comprising latency specific antigens capable of eliciting an immune response, may therefore be combined with Mycobacterial antigens known to elicit immune response in active infections, such as but not limited to: M. tuberculosis antigens ESAT-6 (Rv3875), Ag85A (FbpA/MPT59, Rv3804c), Ag85B (Rv1886c), Ag85C(Rv3803c), CFP10 (Rv3874), TB10.3 (Rv3019c), TB10.4 (Rv0288), MPT64 (Rv1980c), MPT32 (Rv1860) and MPT57 (Rv3418c).

In yet another embodiment, the latency specific antigens and the compositions according to the invention are used to provide methods and reagents to diagnose latent Mycobacterium infections. A method of diagnosing latent or persistent Mycobacterium infections, in particular latent infections, in a subject according to the invention comprises the steps of:

a) contacting a sample of body fluid and/or cells (in particular white blood cells) of the subject, optionally isolated, with one or more polypeptides or fragments thereof selected from the group of Mycobacterium NRP/dormancy (DosR) regulon sequences consisting of Rv0079, Rv0569, Rv1733c, Rv1738, Rv1813c, Rv1996, Rv2007c (FdxA), Rv2029c (PfkB), Rv2030c, Rv2031c (HspX, Acr, 16-kDa alpha crystallin homolog), Rv2032, Rv2626c, Rv2627c, Rv2628, Rv3126c, Rv3129, Rv3130c, Rv3132c, Rv3133c (DosR), Rv0080, Rv1737c (NarK2), Rv1735c and Rv1736c (NarX); and,
b) detecting an immune response to said polypeptides(s) as measured by proliferation or cytokine production being indicative of Mycobacterium infection

The diagnostic method most preferably comprises at least one or more of the polypeptides Rv1733c, Rv2029c (PfkB), Rv2627c, Rv2628, Rv0080, Rv1737c (NarK2), Rv1735c and Rv1736c (NarX). In another preferred embodiment the diagnostic method comprises the detection of an immune response against Rv1733c, Rv2029c (PfkB), Rv2627c and Rv0080 polypeptides, or fragments thereof, which are the most frequently detectable antigens in latent Mycobacterium infections of all latency antigens assayed herein.

A body fluid herein is meant to comprise urine, saliva, semen, tears, lymph fluid and most preferably blood, including blood cells. From blood samples, PBMC's may be obtained and cultured using commonly known techniques. To increase the sensitivity and specificity of the diagnostic method, combined detection of several latency specific antigens is highly preferred, and in a particularly preferred embodiment the method comprises the detection of an immune response against Rv1733c, Rv2029c (PfkB), Rv2627c and Rv0080 antigens. The method may also be combined with detection of other antigens known in the art that are not specific for the latency phase of the Mycobacterium infection such as those specific for the active phase of the infectious process. In the case of M. tuberculosis these antigens may comprise of, but are not limited to: ESAT-6 (Rv3875), Ag85A (FbpA/MPT59, Rv3804c), Ag85B (Rv1886c), Ag85C(Rv3803c), CFP10 (Rv3874), TB10.3 (Rv3019c), TB10.4 (Rv0288), MPT64 (Rv1980c), MPT32 (Rv1860) and MPT57 (Rv3418c).

The invention further provides a diagnostic kit for carrying out the diagnostic method described above, the kit comprising one or more polypeptides or fragments thereof according to the invention and optionally comprises reagents to assay and quantify antibody binding to said polypeptides or measure cellular immune responses. Such reagents may preferably comprise reagents required for the detection of antigen binding, such as but not limited to ELISA or multiplex CBA assays, or reagents for detecting an IFN-γ and/or IL-10 response, such as those used in the examples provided herein. The polypeptides or fragments thereof for detection of Mycobacterial infections may be advantageously attached to a solid carrier such as a protein/peptide (micro-) array or a micro-titer/well plate.

The invention further comprises preferred fragments from the latency specific antigen and/or epitopes comprising polypeptides Rv1733c, Rv2029c (PfkB), Rv2627c, Rv2628, Rv0080, Rv1737c (NarK2), Rv1735c and Rv1736c (NarX). These peptides are preferably used in methods and compositions for eliciting an immune response and for diagnostic purposes according to the invention and are preferably between 18 and 45 amino acids in length and comprising or consisting of the following sequences: VDEPAPPARAIADAALAALG (SEQ ID No. 1) or one of the B and T cell epitopes identified and described herein, in FIGS. 13, 14, 15 and 16.

### DEFINITIONS

Amino acid sequence identity means that two (poly)peptide sequences, when optimally aligned, such as by the programs GAP or BESTFIT using default parameters, share at least a certain percentage of sequence identity as defined elsewhere herein. GAP uses the Needleman and Wunsch global alignment algorithm to align two sequences over their entire length, maximizing the number of matches and minimizes the number of gaps. Generally, the GAP default parameters are used, with a gap creation penalty=8 and gap extension penalty=2. For proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, Calif. 92121-3752, USA. Alternatively percent similarity or identity may be determined by searching against databases such as FASTA, BLAST, etc.

Regulons are, in eukaryotes, genetic units consisting of a noncontiguous groups of genes under the control of a single regulator gene. In bacteria, regulons are global regulatory systems involved in the interplay of pleiotropic regulatory domains and may consist of one or several operons. The NRP/dormancy (DosR) regulon in M. tuberculosis is under control of the DosR transcriptional regulator—(Rv3133c) and comprises at least the 48 sequences described in Voskuil et al. (J. Exp. Med. 2003, 198(5):705-13), listed in table 2.

The term subject as used herein refers to living multi-cellular vertebrate organisms, a category that includes both human and non-human mammals. The term subject includes both human and veterinary or laboratory subjects.

Antigen herein is a property of a molecule, or fragment thereof, that is capable of inducing an immune response in a mammal. The term includes immunogens and regions responsible for antigenicity or antigenic determinants or epitopes. An antigen is a chemical or biochemical structure, determinant, antigen or portion thereof that is capable of inducing the formation of an cellular (T-cell) or humoral (antibody) immune response.

An immune response in vivo or in vitro may be determined and/or monitored by one of the methods provided in this specification, but also by many other methods that are known and obvious to the skilled person and which may for instance be found in Current Protocols in Immunology, Wiley Interscience 2004. A cellular immune response can be determined by induction of the release of a relevant cytokine such as IFN-γ or IL-10 from (or the induction of proliferation in) lymphocytes withdrawn from a mammal, currently or previously infected with (virulent) mycobacteria or immunized with, but not limited to, polypeptide(s). For monitoring cell proliferation the cells may be pulsed with radioactive labeled thymidine or counted in a (flow)cytometer or under a microscope. Induction of cytokines can be monitored by various immuno-chemical methods such as, but not limited to ELISA or Elispot assays. An in vitro cellular response may also be determined by the use of T cell lines derived from a healthy subject or an Mycobacterium-infected mammal where the T cell lines have been driven with either live and/or killed, attenuated or recombinant Mycobacteria, latent Mycobacteria or selected antigens derived or obtained thereof.

The terms vaccine or immunogenic composition is used herein to describe a composition useful for stimulating a specific immune response in a mammal, optionally comprising adjuvants and other active components to enhance or to direct a particular type of immune response, preferably a CTL or Th response.

A latency specific polypeptide or antigen is expressed at higher levels (or exclusively) by a Mycobacterium in its dormant or stationary rather than its active or logarithmic phase of growth, and for M. tuberculosis may be encoded by the NRP/dormancy (DosR) regulon (Voskuil et al, 2003).

A TST positive individual is understood to comprise a mammal with a positive Mantoux test (>5 mm induration) or an individual where Purified Protein Derivative PPD (=tuberculin) induces a positive in vitro recall response determined by release of IFN-γ, and thus may have a latent Mycobacterium infection, i.e. a subject, who has been infected by a (virulent) Mycobacterium, e.g. M. tuberculosis, but shows no sign of (active) disease, such as tuberculosis (TB). A TB patient is understood an individual with culture or microscopically proven infection with (virulent) mycobacteria, and/or an individual clinically diagnosed with TB and who is responsive to anti-TB chemotherapy. Culture, microscopy and clinical diagnosis of TB are well known to any medical practitioner skilled in the art.

A nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

A vector as used herein refers to a nucleic acid molecule as introduced into a host cell thereby producing a transformed host cell. A vector may include nucleic acid sequences that permit it to replicate in a host cell, such as an origin of replication. A vector may for instance be a plasmid, phagemid, phage, cosmid, virus, retrovirus, episome or transposable element. A vector may also include one or more selectable (antibiotic resistance) or visual (e.g. GFP, immuno-tag) marker genes and other genetic elements known in the art.

Proteins, peptides and polypeptides are linear polymeric chains of amino acids (typically L-amino acids) whose alpha carbons are linked through peptide bonds formed by a condensation reaction between the carboxyl group of the alpha carbon of one amino acid and the amino group of the alpha carbon of another amino acid. The terminal amino acid at one end of the chain (i.e., the amino terminal) has a free amino group, while the terminal amino acid at the other end of the chain (i.e., the carboxy terminal) has a free carboxyl group. As such, the term amino terminus (N-terminus) refers to the free alpha-amino group on the amino acid at the amino terminal end of the peptide, or to the alpha amino group (imino group when participating in a peptide bond) of an amino acid at any other location within the peptide. The term carboxy terminus (C-terminus) refers to the free carboxyl group on the amino acid at the carboxy terminal end of a peptide, or to the carboxyl group of an amino acid at any other location within the peptide.

A synthetic polypeptide refers to a polypeptide formed, in vitro, by joining amino acids in a particular order, using the tools of organic chemistry to form the peptide bonds. Typically, the amino acids making up a peptide are numbered in order, starting at the amino terminus and increasing in the direction toward the carboxy terminus of the peptide.

### FIGURE LEGENDS

FIG. 1. Number of long-term T cell lines (n=12) responding to M. tuberculosis latency antigens, hypoxic-M. tuberculosis lysate and culture filtrate (CF). An IFN-γ response of ≧50 pg/ml was considered positive. Lines were generated by stimulating PBMC obtained from TST+ individuals (n=2) or TB patients (n=4) with either lysate (n=4) □ or CF (n=4) [symbol] of M. tuberculosis cultures grown under hypoxic conditions or with the lysate of M. tuberculosis cultures grown under standard, aerated conditions (n=4) [symbol] Bars indicate the number of responding lines to each latency antigen and the number at the top of each bar indicates the median IFN-γ production of these responding lines.

FIG. 2. Number of recognized latency antigens by TST positive individuals (TST+) and TB patients. PBMC's from 23 TST+ individuals and 20 TB patients were stimulated with 25 M. tuberculosis latency antigens. A latency antigen was considered to be recognized when it induced an IFN-γ response of ≧50 pg/ml. For each individual the number of recognized latency antigens was calculated. Bars show the number of individuals recognizing a certain number of latency antigens. (a) TST+ individuals. White bars indicate the number of recent TST converters (total n=12) and black bars the number of remote TST converters (total n=11). (a) TB patients. White bars indicate the number of active TB patients (total n=11) and black bars the number of cured TB patients (total n=9).

FIG. 3. Response profiles to the four best recognized M. tuberculosis latency antigens. IFN-γ production by PBMC's from healthy controls (HC), TB patients (TB) and TST positive individuals (TST+) in response to four M. tuberculosis latency antigens, namely Rv1733c (a), Rv2029c (b), Rv2627c (c), Rv2628 (d). PBMC were also assessed for responsiveness to a lysate (e) or culture filtrate (f) of M. tuberculosis grown under hypoxic conditions. Median values of the subject groups are indicated with a horizontal line. *, P<0.05; **, P<0.01; ***, P<0.001.

FIG. 4. Responses of healthy controls (HC) to M. tuberculosis latency antigens. (a) Healthy non-M. tuberculosis infected controls were divided in two groups on the basis of their response to hypoxic-M. tuberculosis lysate. HC with an IFN-γ response of <100 pg/ml were classified as HC_{low} (n=11) (∘), whereas HC with IFN-γ levels of >100 pg/ml were classified as HC_{high} (n=10) (•). Medians are indicated by a horizontal line. (b) Median IFN-γ responses to the 25 latency antigens were calculated for the HC_{high} group (black bars) and the HC_{low} group (white bars). As shown, IFN-γ responses to latency antigens were nearly exclusively observed in the HC_{high} group (black bars) (b). Individuals in this group are most likely exposed to environmental mycobacteria as indicated by their strong response to hypoxic-M. tuberculosis lysate.

FIG. 5. Proliferation of CFSE labelled CD4 lymphocytes following stimulation by peptides pools of M. tuberculosis Rv1733c. PBMC from a TST+ individual known to respond to the recombinant protein of Rv1733c, were labelled with Carboxy-fluorescein diacetate, succinimidyl ester (CFSE) and stimulated with medium (a), PPD (b), Rv1733c recombinant protein (c) or peptide pools of Rv1733c (d-f). Cells were stained for CD4, followed by assessment of proliferation of CD4-positive cells by measurement of CFSE dilution using a flow cytometer.

FIG. 6. Cytokine profile in response to latency antigens. PBMC of TB patients (n=10) and TST positive individuals (n=10) were stimulated with the indicated latency antigens and hypoxic M. tuberculosis lysate. After 6 days the supernatant was harvested and the cytokine profile determined with the Cytometric Bead Array (BD) using a FACSCalibur flowcytometer. The following cytokines were evaluated: TNFα (red), IL-10 (orange), IL-5 (yellow), 11-4 (green) and IL-2 (blue); median cytokine levels per antigen are indicated for each group. Significant IL-10 responses are observed in TST positives compared to TB patients, in particular for Rv1733c, Rv2029c (PtkB), Rv2627c, Rv2628 and Rv3129.

FIG. 7. Immunogenicity screening of all 48 DosR latency antigens

FIG. 8. Frequency of recognition of the preferred TB latency antigens.

FIG. 9. Euler diagram showing shared responses to TB latency antigens.

FIG. 10. Figure D. Immune responses following BCG vaccination in humans. Individual (i) and median (ii) IFNγ responses of BCG vaccinated individuals to TB latency antigens. BCG vaccinated individuals without any exposure to M. tuberculosis show poor IFNγ production to the TB latency antigens (left), whereas BCG vaccinated individuals that have evidence of exposure to TB (i.e. positive in vitro response to TB specific antigens ESAT6 or CFP10), have a significant production of IFNγ to TB latency antigens (right).

FIG. 11. (Upper panel I) Vaccination of HLA-DR3 transgenic mice with BCG induces poor immune responses to TB latency antigen HspX and its HLA-DR3 restricted T cell epitope, whereas significant responses are observed against Hsp65 and Ag85 recombinant proteins and their HLA-DR3 restricted peptides (as described by Geluk et al. PNAS 95:10797-802).

(Lower panel ii) IFNγ responses to latency TB latency antigens in BALB/c mice. Following BCG vaccination murine splenocytes that are stimulated in vitro with TB latency antigens produce low levels of IFNγ. In contrast, cells stimulated with secreted antigens Ag85A produce significant amounts of IFNγ (A). However, mice are capable of generating immune responses to the tested TB latency antigens: after 3× immunizations with plasmid DNA encoding individual TB latency antigens, splenocytes produce significant amounts of IFNγ (B).

FIG. 12. TMHMM (transmembrane) posterior probabilities analysis of TB antigens.

### EXAMPLES

### Materials and Methods

### Study Subjects

The study included 20 TB patients, 23 healthy tuberculin skin test (TST)-positive individuals and 21 healthy uninfected controls. Since our primary goal was screening of T cell responses to novel latency antigens no assumptions were made with respect to the phase of M. tuberculosis infection in which recognition was most likely to occur. Therefore, a heterogeneous set of TB patients and TST converters were chosen as study subjects.

Of the 20 TB patients, 11 had active TB disease, who were treated between 2 weeks and 6 months (mean 2.5 months) and 9 with cured TB who were treated between 4-63 years before blood sampling (mean interval 29 years). Eleven patients had pulmonary and 9 had extra-pulmonary TB. The mean age at the time of blood sampling was 46 years (range 17-75), 14 were male. Nine patients were of Dutch origin, 3 were North Africans, 6 were Africans and 3 were of Asian descent. None of the patients had risk factors for HIV.

The 23 TST-positive persons were all healthy, non-BCG-vaccinated, individuals with a documented TST result of ≧10 mm induration, mostly after contact with a case of smear-positive pulmonary TB (n=14). The mean age was 37 year (range 21 to 63), 14 were male. All were of Dutch origin. From 12 persons, blood was drawn within 6 months after TST conversion, of whom only 5 were treated with isoniazid for latent TB infection. In the remaining 11 TST-positive individuals, the mean interval between conversion and blood sampling was 6 years (range 2 to 12 years). Only 2 of these remote TST converters had received isoniazid. Up to the time of this writing, none of the TST-positive individuals developed active TB disease after a mean period of 4.9 years after TST conversion. Most of the individuals in this group can be regarded as latently infected individuals, who show a certain level of natural protection against the development of active TB disease.

As a control group, 21 healthy, non-BCG vaccinated individuals were studied. None of the healthy controls had any known exposure to TB. They were either TST negative (n=18; in the remaining others no TST was done) and or were tested negative for the M. tuberculosis specific proteins, ESAT-6 and CFP-10 by ELISPOT for IFN-γ (21). All controls were of Dutch origin with an average age of 30 years (range 22-44 year), 7 were male.

Blood samples were obtained from all study subjects by standard venous puncture using heparinized tubes after written informed consent was obtained. Subsequently PBMC were isolated using a Ficoll density gradient and stored in liquid nitrogen, as described previously (22). The study protocol (P207/99) was approved by the institutional review board of the Leiden University Medical Center.

M. tuberculosis Antigens and Peptides

For the present study, M. tuberculosis H37Rv was grown for 24 hours in tubes with tightly screwed caps, harvested and lysed as previously described (16). This lysate, which will be further referred to as hypoxic-lysate, was precipitated with acetone and dialysed against PBS. The culture filtrate of this low oxygen culture, was concentrated with a centriprep-concentrator. The protein concentration of the resultant preparations was determined by BCA test (Pierce, Rockford, Ill.). The hypoxic-lysate and hypoxic-culture filtrate were kindly provided by the Statens Serum Institute (Copenhagen, Denmark). A lysate from M. tuberculosis, cultured under standard aerated laboratory conditions was provided by the National Institute of Public Health and Environment (Bilthoven, the Netherlands).

Recombinant proteins were made of the 25 most upregulated genes from the dormancy regulon of M. tuberculosis (Table I). Genes were amplified by PCR and cloned by Gateway Technology (Invitrogen, San Diego, Calif.) in a bacterial expression vector containing an N-terminal histidine tag. The proteins were overexpressed in Escherichia coli B strain BL21(DE3) and purified as previously described (23). To confirm that the correct sequence was expressed, all inserts were sequenced. Size and purity were checked by gel eletrophoresis and Western blotting with anti-His antibodies. Residual endotoxin levels were less than 50 I.U./mg protein as assessed by Limulus Amebocyte Lysate test (BioWhittaker, Walkersville, Md.).

Twenty synthetic peptides from latency antigens Rv1733c, Rv2029c, Rv2627c and Rv2628 were produced each 20 amino acids (aa) long, with 10 aa overlap and spanning the complete aa sequence of the said latency antigens (22). The sequence of all peptides was elongated with two lysine residues at the C-terminus to improve solubility. For optimal use of PBMC's, we choose to generate 9 pools of peptides, each with 4 to 5 peptides and with each individual peptide being present in 2 different pools. This method enabled the identification of the specific peptide within a pool that was recognized by antigen specific T cells.

### T Cell Lines

Eight long-term T cell lines were generated against either lysate (n=4) or culture filtrate (n=4) of M. tuberculosis grown under low oxygen conditions, using PBMC obtained from two TB patients and two TST positive individuals known to respond to HspX. For comparison, 4 additional M. tuberculosis-specific T cell lines where made by stimulating PBMC from three TB patients and from one TST-positive individual with lysate from M. tuberculosis cultured under standard aerated laboratory conditions. T cell lines were generated as previously described (24). In short, PBMC were incubated at 1×10⁶ cells/well in 24-well plates (Nunc, Roskilde, Denmark) in the presence of 5 μg/ml antigen as specified above. After 6 days, 25 U/ml interleukin-2 (Cetus, Amsterdam, The Netherlands) was added and cultures were continued for another 2 to 3 weeks. T cells were frozen and stored in liquid nitrogen until use.

### T Cell Proliferation Assay

T cells (5×10⁴/well) were cultured with autologous or HLA-DR matched irradiated PBMC as antigen presenting cells (1.5×10⁴/well) in triplicate, in 96-wells flat-bottomed microiter plates (NUNC) in the presence or absence of antigen. Iscoves modified DMEM (Gibco, Paisley, Scotland) supplemented with 10% pooled human serum, 40 U/ml penicillin and 40 μg/ml streptomycin was used as standard culture medium. All 25 recombinant latency antigens, as shown in Table I, were tested at a final concentration of 0.33 μM and the standard M. tuberculosis lysate, the hypoxic-lysate and -culture filtrate at a concentration of 1 μg/ml. The mitogen PHA (2 μg/ml) was used as a positive control. After three days of culture at 37° C. and 5% CO₂, supernatants (50 μl/well, pooled per triplicate) were collected for the detection of IFN-γ and proliferation of T cells was measured by [³H] thymidine incorporation as previously described (22). Proliferation was expressed as stimulation index, calculated as counts per minute in stimulated wells divided by the counts per minute in unstimulated wells. A stimulation index of ≧4 was predefined as a positive response.

### Lymphocyte Stimulation Assay

PBMC (1.5×10⁵/well) were cultured in standard culture medium in triplicate in 96-wells round-bottom microliter plates at 37° C., 5% CO₂, in the absence or presence of latency antigens. The same antigens and the same batches were used throughout all experiments. Due to a shortage of the Rv1733c batch the number of study subjects for this antigen was restricted to 17 healthy controls, 18 TST+ persons and 16 TB patients. At day 6, supernatants were harvested (75 μl/well, pooled per triplicate) for the detection of IFN-γ and proliferation of T cells was measured as described elsewhere (22).

### IFN-γ Detection

IFN-γ concentration in the supernatants was measured by ELISA (U-CyTech, Utrecht, The Netherlands). The detection limit of the assay was 20 pg IFN-γ/ml. ELISA samples were tested in duplicate. The mean value of unstimulated cultures was subtracted from the mean value of the stimulated cultures. A positive response was predefined as an IFN-γ level of ≧50 pg/ml in the supernatants from the stimulated T cell lines and of ≧100 pg/ml from the PBMC cultures.

### Multiplex Cytokine Detection

Levels of IFNγ TNFα, IL-10, IL-5, IL-4 and IL-2 in the culture supernatants were determined using the Cytometric Bead Array (CBA) kit for human Th1/Th2 cytokine (BD Biosciences), which allows simultaneous detection of multiple cytokines in one sample. Assays were performed according to manufacturer's instructions.

### Proliferation of CFSE Labelled PBMC's

PBMC were thawed and resuspended in PBS/0.5% BSA (37° C.) at 10×10⁶ cell/ml. CFSE was added in a final concentration of 5 μM and incubated 10 min at 37° C. in the dark. After incubation, FCS (10%) was added and cells were washed twice in PBS/0.5% BSA. Labelled PBMC (1×10⁶ cell/well) were cultured in 24-wells plates in standard culture medium in the presence of either PPD (5 μg/ml), Rv1733c recombinant protein (20 μg/ml), Rv1733c peptide pools (10 μg/ml per peptide), PHA (2 μg/ml) or medium alone. After 6 days, cells were washed with PBS/0.1% BSA and stained for CD4 followed by assessment of proliferation of CD4 positive cells by measurement of CFSE dilution using a flow cytometer.

### Statistical Analysis

For the comparison of the proportion of responders in each study groups, the chi-square test was used. Median IFN-γ responses were evaluated non-parametrically using a Kruskal-Wallis test for comparison of all three study groups and Mann-Whitney U test was used as post-hoc test when two groups were compared. Since the primary aim of the study was the initial screening of potential immunogenic latency antigens, the Bonferroni correction was not applied. The non-parametric Friedman's test (variance by ranks) was applied to test the hypothesis that TST positive individuals would recognize the 25 latency antigens in general better than TB patients. A P value of <0.05 was considered statistically significant. For the statistical analysis SPSS 10.0 for Windows was used.

### Example 1

### Selection of Immunogenic Latency Antigens

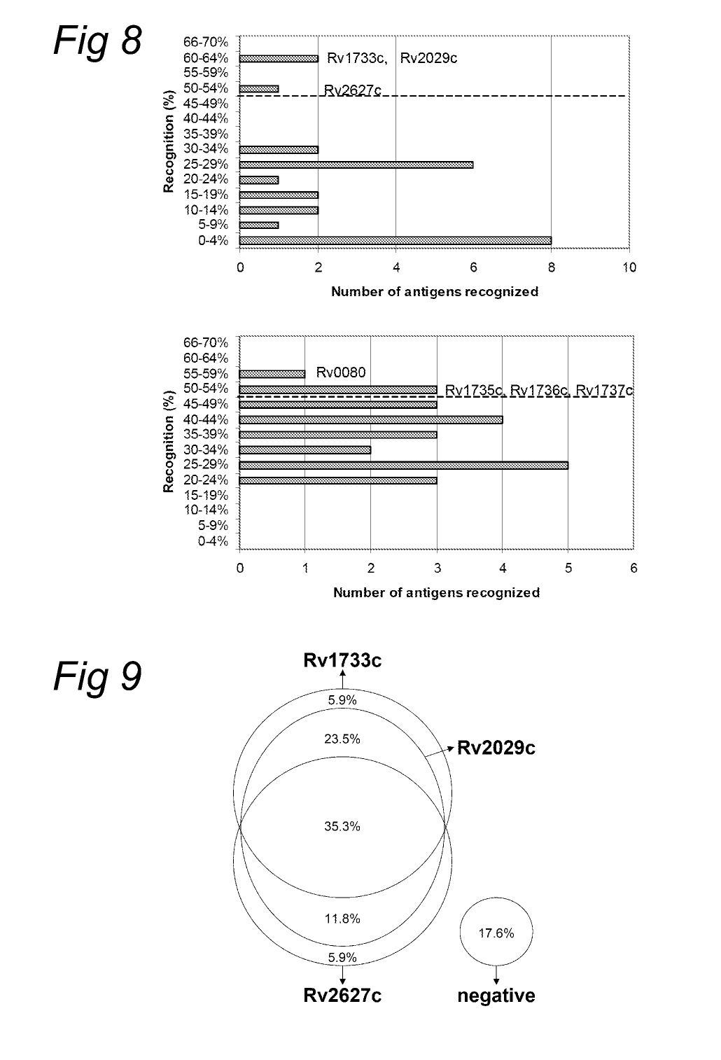

Antigens were selected from the recently-identified dormancy regulon of M. tuberculosis, consisting of 48 genes (table 2) which were found to be induced during NRP, oxygen limitation and during low dose nitric oxide exposure (17). As most of these genes are hypothetical open reading frames with unknown function a selection of the genes for this post-genomic antigen discovery project could not be based on protein function. Therefore, we chose to select the genes on their level of induction. For this purpose, a mean fold induction was calculated for each individual gene, based on the fold inductions as observed by Voskuil et al. in the three different in vitro models of latency (17). Of the data from 48 candidate genes, the 25 most strongly induced genes were selected for cloning and expression of recombinant proteins (Table 1; FIGS. 7i and 8i). These hypothetical proteins were subsequently tested in equal molar concentrations in order to allow for a direct comparison between latency antigens which vary considerably in size (9 to 74 kDa). The 25 antigens are all recognized by short-term T cell lines generated with M. tuberculosis sonicate.

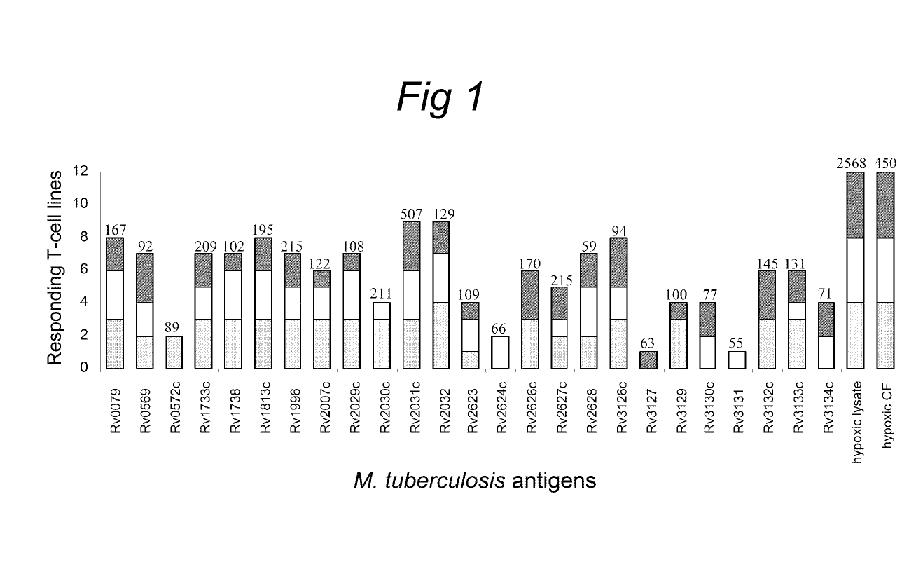

For the initial assessment of the immunogenicity of these hypothetical latency antigens, long-term T cell lines were generated against either lysate (n=4) or culture filtrate (n=4) of M. tuberculosis grown under low oxygen conditions or standard aerated conditions (n=4) and specificity confirmed by T cell proliferation assays. Importantly, all 25 latency antigens were recognized (IFN-γ>50 pg/ml) by at least 1 of the 12 T cell lines tested and 20 antigens by at least 4 T cell lines (FIG. 1). Latency antigens HspX (Rv2031c) and Rv2032 were most frequently recognized, by 75% of tested T cell lines, with median IFN-γ levels of 507 and 129 pg/ml respectively among responding lines. Most latency antigens were recognized by T cell lines raised with hypoxic-lysate as well as by those generated against hypoxic culture filtrate, indicating that latency antigens can also be found extra-cellular, in the culture filtrate. This confirms a previous study showing that Rv0569, Rv2623 and Rv2626c proteins were present in culture filtrate of M. tuberculosis grown under hypoxic conditions (16). The standard-lysate specific T cell lines responded equally well to the latency antigens as did the hypoxic-lysate specific T cell lines (FIG. 1). This finding is in agreement with the recent observation that latency antigens are also expressed by M. tuberculosis in standard aerated cultures when bacteria are harvested during the stationary growth phase, although to a lesser extent than when cultured under defined hypoxic conditions (25). Western blot analysis of the standard-aerated-M. tuberculosis lysate which we used to generate the T cell lines, confirmed the presence of latency antigen HspX in this preparation (data not shown). Similar results from the proliferation data (data not shown) confirmed the responsiveness of T cell lines to latency antigens. This first explorative screening demonstrated that all 25 novel mycobacterial latency antigens were potentially able to elicit a cellular immune response.

### Example 2

### Interferon-γ Production by PBMC in Response to M. tuberculosis Latency Antigens

Subsequently, the 25 latency antigens were studied for the induction of IFN-γ production by PBMC of 20 TB patients, 23 TST positive healthy individuals and 21 uninfected control subjects. For each individual latency antigen, the proportion of responding (IFN-γ≧100 pg/ml) study subjects per group was calculated (Table I). We also calculated the proportion of responders among all 43 M. tuberculosis infected individuals, taking together 20 TB patients and 23 TST positive individuals. The latter analysis showed that 19 latency antigens were recognized by at least 5% of the M. tuberculosis infected individuals, with Rv1733c being recognized by the majority (56%) of the infected individuals. The remaining 6 antigens tested, Rv0572c, Rv2623, Rv2624c, Rv3127, Rv3131, Rv3134c were not or very poorly recognized by M. tuberculosis infected individuals. Similar recognition profiles were found when proliferation data were analysed (not shown).

TABLE 1
Immunogenicity of latency antigens^{a}

| | | | Responders (%)^{b} | | |
|---|---|---|---|---|---|
| | | | HC | TB | TST+ |
| Rv no. | Gene | Product | (n = 21) | (n = 20) | (n = 23) |
| Rv0079 | | HP | 14 | 10 | 26 |
| Rv0569 | | CHP | — | 5 | 22 |
| Rv0572c | | HP | 10 | 5 | — |
| Rv1733c | | possible | 41 | 50 | 61 |
| | | transmembrane protein | | | |
| Rv1738 | | CHP | — | 11 | 13 |
| Rv1813c | | CHP | 14 | 15 | 17 |
| Rv1996 | | CHP | 14 | 11 | 4 |
| Rv2007c | fdxA | probable ferredoxin A | — | — | 9 |
| Rv2029c | pfkB | probable | 29 | 25 | 61* |
| | | phosphofructokinase B | | | |
| Rv2030c | | CHP | 14 | 15 | 26 |
| Rv2031c | hspX | heat shock protein | 5 | 20 | —* |
| | | HspX | | | |
| Rv2032 | acg | CHP Acg | 14 | 20 | 30 |
| Rv2623 | TB31.7 | CHP TB31.7 | — | — | — |
| Rv2624c | | CHP | — | — | — |
| Rv2626c | | CHP | 14 | 10 | 30 |
| Rv2627c | | CHP | 38 | 30 | 52 |
| Rv2628 | | HP | 10 | 16 | 35 |
| Rv3126c | | HP | 19 | 10 | 30 |
| Rv3127 | | CHP | — | — | 4 |
| Rv3129 | | CHP | 19 | 21 | 35 |
| Rv3130c | | CHP | — | — | 13 |
| Rv3131 | | CHP | — | — | 4 |
| Rv3132c | devS | sensor histidine kinase | 24 | 15 | 17 |
| Rv3133c | dosR | transcriptional | 14 | 32 | 30 |
| | | regulatory protein | | | |
| Rv3134c | | CHP | — | — | 4 |

^{a}Abbreviations: HC, healthy controls; TB, TB patients; TST+, tuberculin skin test positive individuals; HP, hypothetical protein; CHP, conserved hypothetical protein. Annotations are from http://genolist.pasteur.fr/TubercuList/
^{b}IFN-γ response of ≧100 pg/ml was considered positive.
—, none of the subjects responded
*P < .05, χ² test comparing TB patients with TST+ individuals.

In addition to the 25 most strongly induced DosR genes, the entire group of 48 DosR genes was tested for INFγ production again, including the remaining 23 TB latency antigens not yet tested. All DosR genes were tested in a similar fashion as described above, results are shown in FIG. 7ii and 8ii. From the lower panel it is clear 4 additional antigens could be selected as potential vaccine candidates: Rv0080, Rv1737c (NarK2), Rv1735c and Rv1736c (NarX), yielding a strong INFγ induction, in addition to Rv1733c, Rv2029c, Rv2627c and Rv2628.

### Example 3

### TB Patients and TST Positive Individuals Respond Differently to Latency Antigens

Median IFN-γ responses in the group of TB patients and TST converters were determined for each latency antigen. Considering the 25 latency antigens as a group, the median IFN-γ responses were consistently and significantly higher in TST positive individuals, who are considered to be latently infected with M. tuberculosis, than in TB patients (P<0.01; Friedman's test). To analyze this difference in antigen recognition further, we calculated the ratio between the IFN-γ response to each latency antigen and the total response to hypoxic-M. tuberculosis-lysate in the same individual. This analysis corrects for a possible inter-individual variation in the general responsiveness of T cells. When the above Friedman analysis was repeated, now comparing the medians of these ratio's, it was confirmed that latency antigens were preferentially recognized by TST positive individuals (P<0.01).

When comparing the proportions of responders in the group of TST positive individuals and the group of TB patients for each individual latency antigen, it was found that nearly all latency antigens were recognized by a larger proportion of TST positive individuals compared to TB patients (Table I). However, this trend was only significant (P=0.02) for Rv2029c, that was recognized by 61% of TST positive individuals and 25% of TB patients.

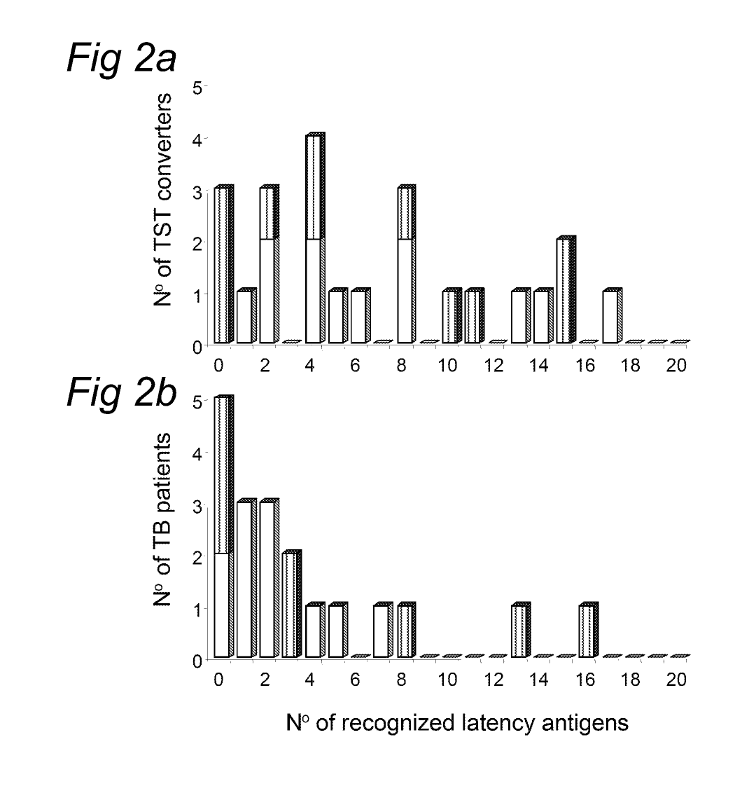

Response profiles differed between individuals, both with regard to the number of latency antigens and the specific antigens that were recognized (FIG. 2). TST positive persons recognized a median of 4 out of the 25 latency antigens tested, while in contrast TB patients recognized a median of only one latency antigen.

In FIG. 8 an overview is given of the frequency of recognition of TB latency antigens by Mantoux positive individuals. For the first series of 25 TB latency antigens, it appeared that not all antigens were equally recognized (Cochran's Q test, P<0.001). Based on that notion we selected the latency antigens with at least 50% recognition in the first series: Rv1733c, Rv2029c (PfkB), Rv2627c and Rv2628 and from the second series of 23 TB latency antigens with the same characteristics: Rv0080, Rv1737c (NarK2), Rv1735c and Rv1736c (NarX) were selected. These 8 antigens provide a specific subset of TB latency antigens that are most suitable for diagnostic and vaccination purposes, as individual antigens, or fragments thereof, but most preferably used in combination of 1, 2, 3, 4, 5, 6, 7 or all 8 DosR gene-products selected from the group consisting of Rv1733c, Rv2029c, Rv2627c, Rv2628, Rv0080, Rv1737c (NarK2), Rv1735c and Rv1736c (NarX).

### Example 4

### Interferon-γ Responses to Frequently Recognized Latency Antigens

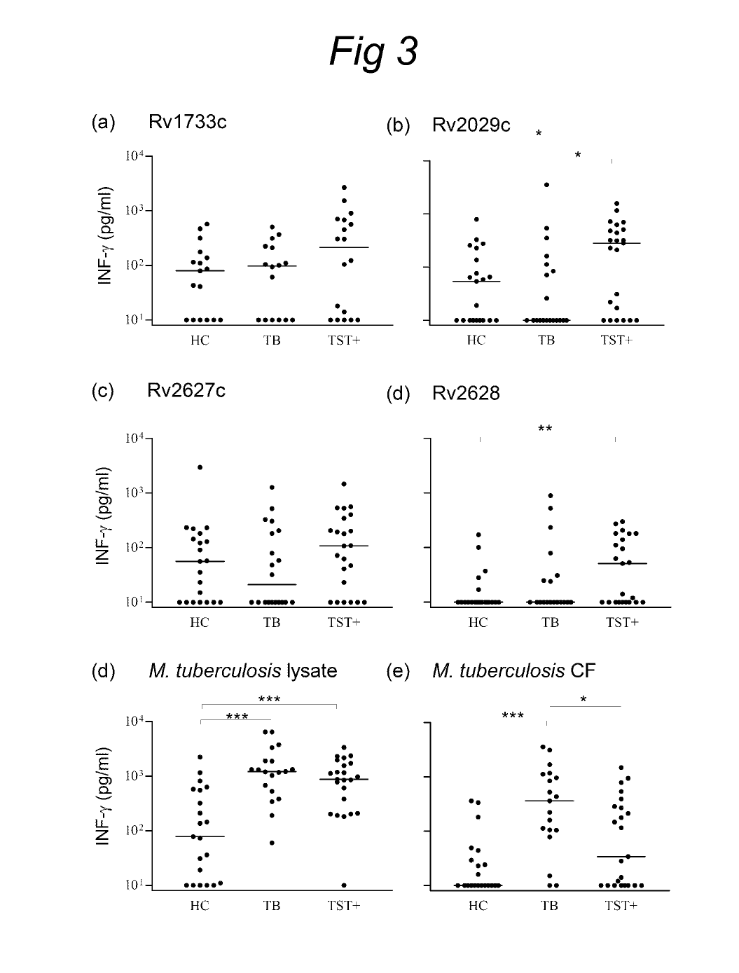

Four latency antigens, namely Rv1733c, Rv2029c, Rv2627c and Rv2628 were broadly recognized by predominantly latently infected individuals and induced the strongest Th1 response, as measured by IFN-γ production. For each study group the IFN-γ responses to these 4 antigens are shown in FIG. 3. The median IFN-γ production in the group of TST positive persons in response to Rv1733c, Rv2029c, Rv2627c and Rv2628 were 213, 281, 107 and 51 pg/ml, respectively. In contrast, much lower IFN-γ responses to these four antigens were found in TB patients, with medians of 98, 16, <10 and <10 pg/ml, respectively. This difference in IFN-γ response was statistically significant (P=0.03) for antigen Rv2029c (FIG. 3).

Surprisingly, one latency antigen, Rv2031c, was recognized (IFN-g>100 pg/ml) significantly more frequently by TB patients than by TST converters (P=0.02) (Table I). However, when a quantitative analysis was done, directly comparing the IFN-γ production between the 2 groups no statistically significant difference could be found. Interestingly, the TST converters who showed at least some response to Rv2031c (IFN-γ levels between 20-100 pg/ml) were all recently TST converted (<6 month) and thus were only recently exposed to M. tuberculosis.

Another interesting latency antigen we studied is Rv3133c/dosR, that was shown to act as a transcription factor mediating the hypoxic response of M. tuberculosis (15,26,27). Recently a Rv3133c/dosR mutant strain was studied; it showed reduced pathological changes and bacterial burden in guinea pigs but did not alter the entry, survival and multiplication of M. tuberculosis in human monocytes in vitro (28). In our study, Rv3133c was recognized by approximately one third of the TST positive individuals and of the TB patients, with median IFN-γ responses among responders of 227 and 145 pg/ml, respectively.

In FIG. 9 (Euler diagram) the overlap in IFNγ responses is represented for the best 4 TB latency antigens of the first series for subjects in which all individual antigens were evaluated: 82.4% of Mantoux positive individuals is responding to one or more of the following latency antigens: Rv1733c, Rv2029c (PfkB), Rv2627c and Rv2628. The combination of Rv1733c and Rv2627c is most frequently recognized, and exclusively responsible for this phenomenon; with no added value of Rv2029c (PfkB) and/or Rv2628. Rv2029c (PtkB) is the most frequently recognized (70.6%) TB latency antigen among Mantoux positives. A combination of these antigens, preferably in combination with Rv0080, Rv1735c, Rv1736c and/or Rv1737c would be highly suitable and preferred for diagnostic testing and for the composition of a latency and/or a multistage vaccine.

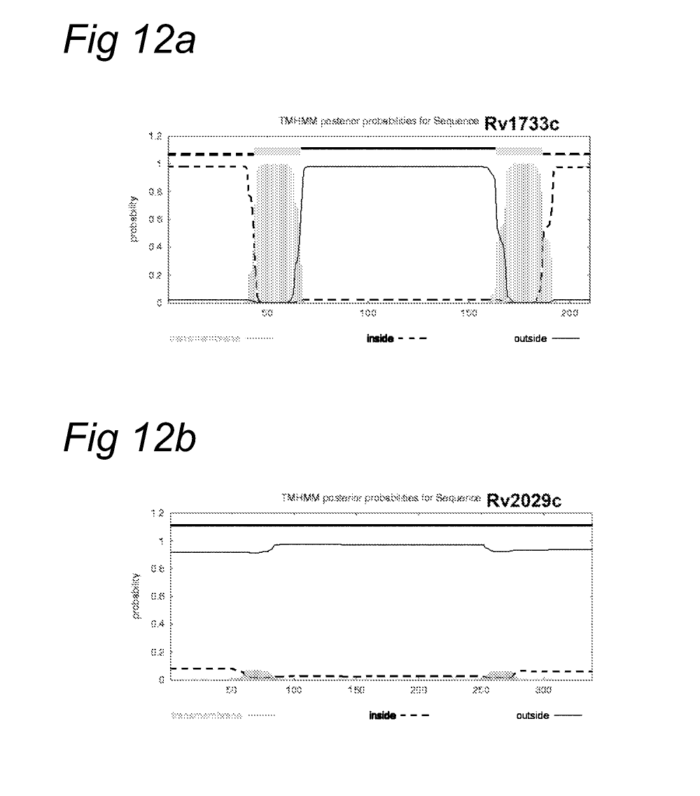

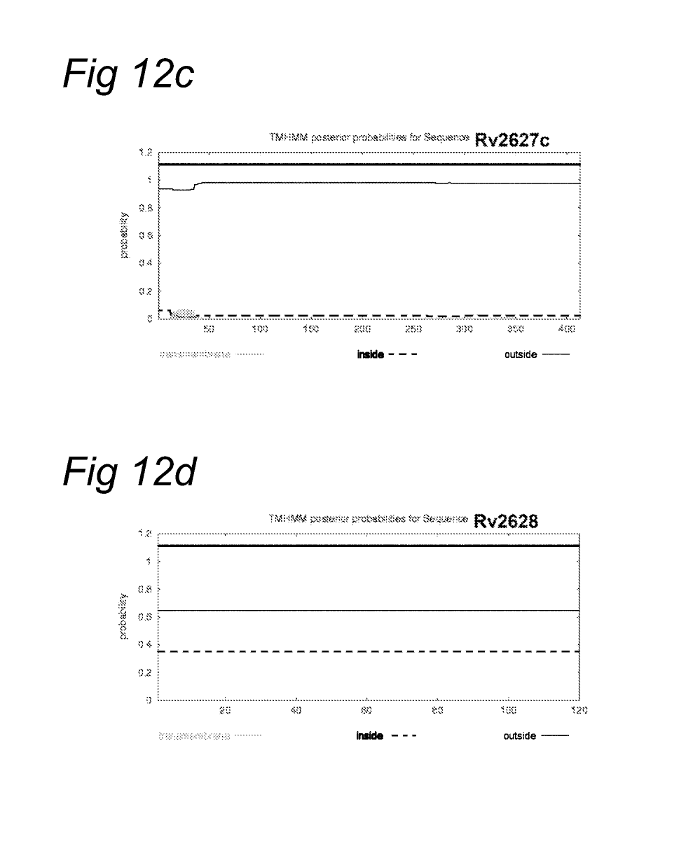

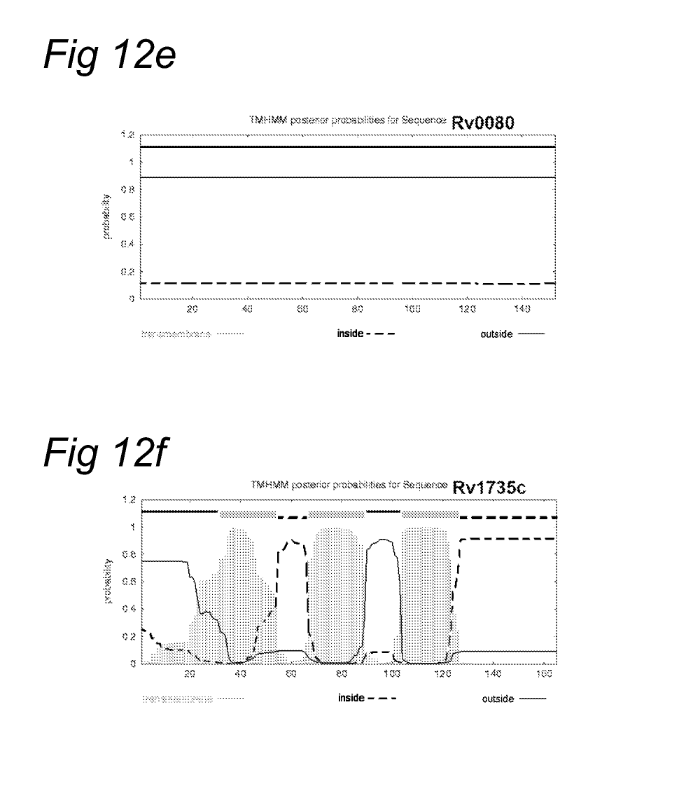

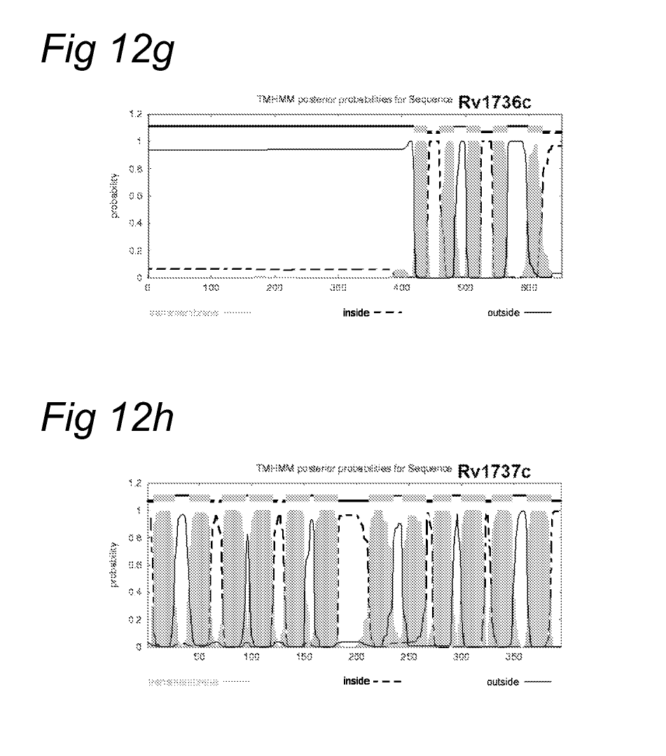

In FIG. 12 the prediction of transmembrane helices are displayed for TB latency antigens Rv1733c, Rv2029c (PfkB), Rv2627c, Rv2628, Rv0080, Rv1737c (NarK2), Rv1735c, and Rv1736c (NarX). The predictions were done using TMHMM 2.0 Server. Krogh A, Larsson B, von Heijne G, Sonnhammer E L. Predicting transmembrane protein topology with a hidden Markov model: application to complete genomes. 2001. J Mol. Biol. 305(3):567-80 (http://www.cbs.dtu.dk/services/TMHMM). In combination with predicted data MHC class I and II epitopes in tables 3 and 4 and in vitro data in Table 5, this analysis demonstrates that CD8 T cell responses to TB antigens can be detected irrespective of the fact that these antigens are secreted or membrane bound (Klein M R et al., HLA-B*35-restricted CD8 T cell epitope in Mycobacterium tuberculosis Rv2903c. 2002 Infect Immun. 70(2):981-4).

### Example 5

### Recognition of Latency Antigens by Healthy Controls

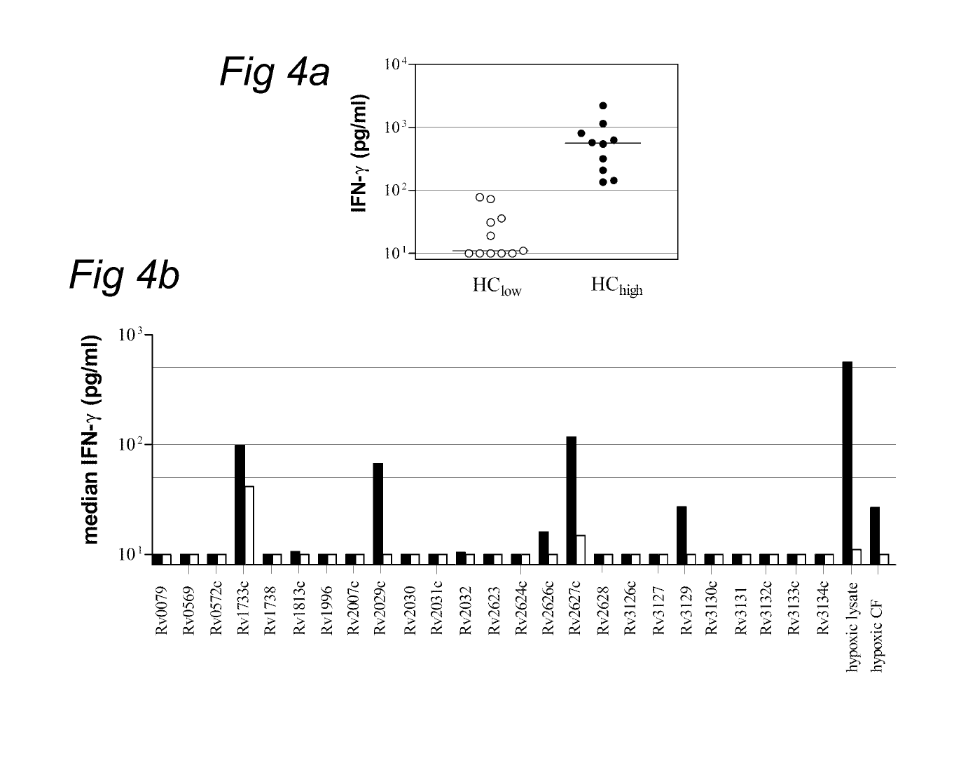

Somewhat unexpectedly, 16 of the 25 latency antigens were recognized by T cells of a minority of healthy individuals (Table I), although the strength of the immune response was generally lower than in M. tuberculosis infected individuals. Since all healthy non-BCG vaccinated controls were TST negative and in vitro responses to the M. tuberculosis-specific immunodominant antigens ESAT6 and CFP10 were also negative in this group (data not shown), we concluded that the observed responses to latency antigens were not caused by infection with M. tuberculosis complex species. However, 10 of 21 (48%) healthy controls did respond significantly to hypoxic-M. tuberculosis lysate, with a median IFN-γ response among responders of 563 pg/ml (FIGS. 3 and 4). This finding is compatible with T-cell cross-reactivity to mycobacterial antigens resulting from previous exposure to environmental mycobacteria. Since responses to latency antigens were predominantly observed in this group of healthy controls who strongly responded to hypoxic-M. tuberculosis lysate (FIG. 4), these responses are most likely also reflecting previous exposure to cross-reactive environmental mycobacteria. Only Rv1733c was also sometimes recognized by healthy controls who did not respond strongly to hypoxic-M. tuberculosis lysate, with a median IFN-γ of 41 pg/ml, suggesting possible cross-reactivity to antigens other than mycobacterial antigens.

### Example 6

### Proliferation of CFSE Labelled PBMC in Response to Peptides of Rv1733c

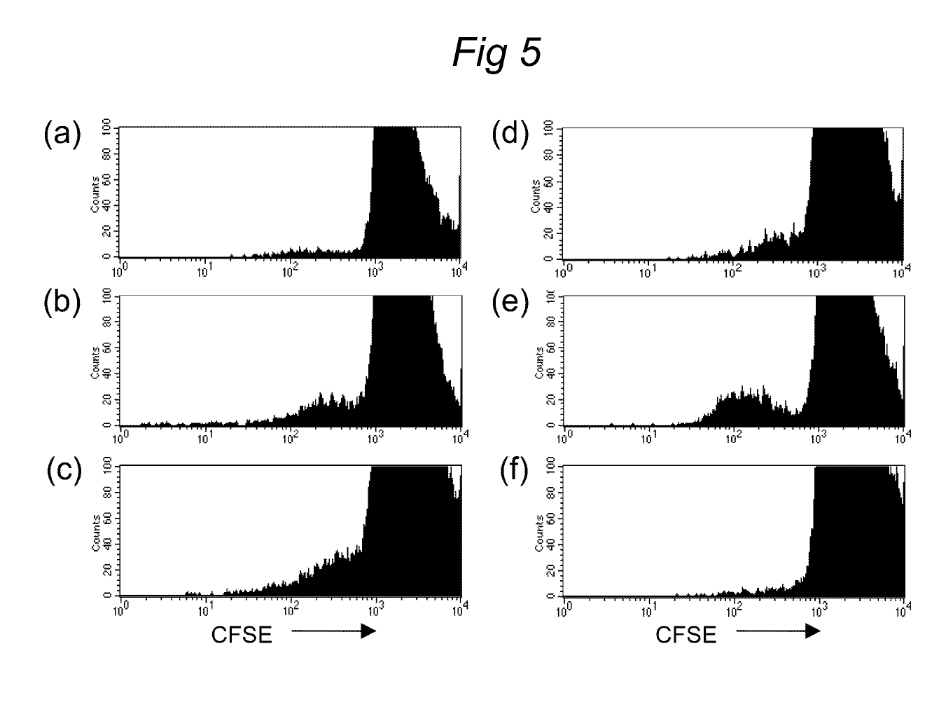

In order to confirm protein specific activation by the latency antigens, we determined peptide specific proliferation of Rv1733c as the most frequently recognized antigen. PBMC from TST positive individuals and a healthy control, known to respond to Rv1733c, were CFSE labelled and stimulated with recombinant protein or peptide pools of Rv1733c. After 6 days cells were stained for CD4 and proliferation of CD4 T cells was assessed using a flow cytometer. Stimulation with PPD and Rv1733c recombinant protein both induced strong proliferation of CD4+ T cells. Also several peptide pools of Rv1733c, in particular the pools containing peptide 16, were able to induce proliferation of CD4+ T cells. This is shown in FIG. 5 for PBMC from a TST positive individual. In silico prediction of epitopes of Rv1733c for the HLA type of this donor (DRB1*15) resulted in the finding of several possible epitopes, of which two are located in peptide 16 (VDEPAPPARAIADAALAALG and VDEPAPPARAIADAALAALG), which is in accordance with the observed proliferation of CD4 T cells in response to peptide 16. Peptides of Rv1733c also induced proliferation of CD4 cells using PBMCs from a healthy control, who responded to the recombinant protein of Rv1733c, which indicates that this response is antigen specific.

In addition to peptide-16 from Rv1733c that was mentioned in the original application as a sequence containing T cell epitopes, the following additional T and B cell epitopes and peptides have now been determined in the selected TB latency antigens: In Table 3 and Table 4 all predicted HLA class-I and -II restricted T cell epitopes are represented for the best 4 TB latency antigens of the first series (i.e. Rv1733c, Rv2029c (PfkB), Rv2627c and Rv2628) and of second series (i.e. Rv0080, Rv1737c (NarK2), Rv1735c and Rv1736c (NarX)) respectively.

Nine-mer amino acid sequences are listed in Table 4 that represent predicted CD8 T cell epitopes restricted by all known HLA class-I supertypes: A1, A2, A3, A24, B7, B8, B27, B44, B58 and B62.

The predictions were made using NetCTL 1.0 Server, which predicts CD8 T epitopes in protein sequences. The method integrates prediction of peptide MHC binding, proteasomal C terminal cleavage and TAP transport efficiency. The server allows for predictions of CTL epitopes restricted to 10 HLA supertypes. MHC binding and proteasomal cleavage is performed using artificial neural networks. TAP transport efficiency is predicted using weight matrix. Reference: Larsen M V et al., 2005. Eur J Immunol 35(8): 2295-303 (www.cbs.dtu.dk/services/NetCTL).

Twenty-mer amino acid sequences are listed (Table 4) that represent predicted CD4 T cell epitopes restricted by 25 HLA class-II alleles: DR1 (DRB1*0101, *0102), DR3 (DRB1*0301), DR4, (DRB1*0401, *0402, *0404, *0405, *0410, *0421), DR7 (DRB1*0701), DR8 (DRB1*0801, *0802, *0804, *0806), DR11(5) (DRB1*1101, *1104, *1106, *1107), DR13(6) (DRB1*1305, *1307, *1307, *1321), DR15(2) (DRB1*1501, *1502), and DRB5*0101. The predictions were made using TEPITOPE 2000 (Vaccinome). TEPITOPE is a T cell epitope prediction model based on HLA class-II peptide binding and allows for rapid identification of promiscuous HLA class-II ligands and epitopes in sets of protein sequences. Reference: Bian H, Hammer J. Discovery of promiscuous HLA-II-restricted T cell epitopes with TEPITOPE. 2004. Methods. 34(4):468-75.

In Table 5 all 20-mer peptides are listed derived from Rv1733c, Rv2029c (PfkB), Rv2627c and Rv2628 that were tested for recognition in twenty PPD positive donors. Cells were labeled with CFSE, stimulated with peptide, recombinant protein or control antigens. Proliferation of CD4 and CD8 T cells was measured by flowcytometry and supernatants were harvested and analyzed by multiplex cytokine assays.

Indicated in red are peptides that gave strong proliferation (>75%) of CD4 or CD8 T cells in multiple donors, in green peptides with >50-75% proliferation, and in light green peptides with >20-50% proliferation (Table 5). Similar data is expected for the other TB latency antigens (i.e. Rv0080, Rv1737c (NarK2), Rv1735c and Rv1736c (NarX)).

In Table 6 the amino acid sequences are listed of Rv1733c, Rv2029c (PfkB), Rv2627c, Rv2628, Rv0080, Rv1737c (NarK2), Rv1735c and Rv1736c (NarX) and indicated in bold and underlined are linear and conformational predicted B cell epitopes respectively. Predictions were made using BepiPred 1.0 Server, which predicts the location of linear B-cell epitopes using a combination of a hidden Markov model and a propensity scale method. Conformational B cell epitopes were only predicted for TB latency antigens with sequence homology to other known proteins with available structure and functional data (i.e. Rv2029c (PfkB), Rv1736c (NarX) and Rv1737c (NarK2). (Ref: Larsen, J E P, Lund O, Nielsen M. 2006, Improved method for predicting linear B-cell epitopes (http://www.cbs.dtu.dk/services/BepiPred).

### Example 7

### Production of Other Cytokines in Response to Latency Antigens

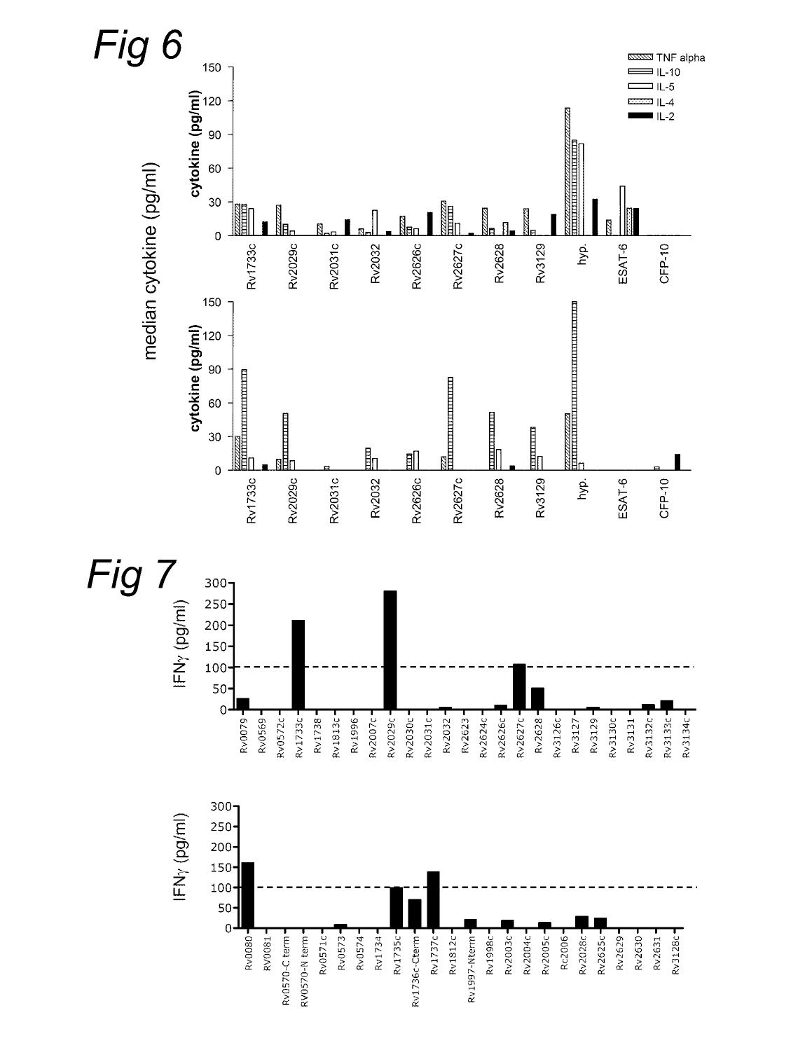

In addition to IFNγ by ELISA (examples 1-6), a series of other cytokines were measured in day-6 supernatants of PBMC stimulated with recombinant antigens. PBMC of TB patients (n=10) and of Mantoux positive individuals (n=10) were stimulated with recombinant proteins Rv1733c, Rv2029c (PtkB), HspX (Rv2031c), Rv2032, Rv2626c, Rv2627c, Rv2628, Rv3129, ESAT-6 and CFP10, and in addition as positive control the hypoxic lysate of M. tuberculosis (FIG. 6). CBA data for IFNγ confirmed observations made by ELISA. TNFα and IL-5 responses were found in both groups without apparent skewing. Very poor responses were detected for IL-2 and IL-4; and if there were any detectable responses they were observed in TB patients. Interestingly, for a number of latency antigens we observed significant IL-10 responses in Mantoux positives and not in TB patients (FIG. 6).

TABLE 2
M. tuberculosis DosR regulon (AL123456)

| | | Size | |
|---|---|---|---|
| H37Rv | gene | (aa.) | Description |
| Rv0079 | | 273 | HYPOTHETICAL PROTEIN |
| Rv0080 | | 152 | CONSERVED HYPOTHETICAL PROTEIN |
| Rv0081 | | 114 | PROBABLE TRANSCRIPTIONAL REGULATORY PROTEIN |
| Rv0569 | | 88 | CONSERVED HYPOTHETICAL PROTEIN |
| Rv0570 | nrdZ | 692 | PROBABLE RIBONUCLEOSIDE-DIPHOSPHATE REDUCTASE |
| Rv0571c | | 443 | CONSERVED HYPOTHETICAL PROTEIN |
| Rv0572c | | 113 | HYPOTHETICAL PROTEIN |
| Rv0573c | | 463 | CONSERVED HYPOTHETICAL PROTEIN |
| Rv574c | | 380 | CONSERVED HYPOTHETICAL PROTEIN |
| Rv1733c | | 210 | PROBABLE CONSERVED TRANSMEMBRANE PROTEIN |
| Rv1734c | | 80 | CONSERVED HYPOTHETICAL PROTEIN |
| Rv1735c | | 165 | HYPOTHETICAL MEMBRANE PROTEIN |
| Rv1736c | narX | 652 | PROBABLE NITRATE REDUCTASE |
| Rv1737c | narK2 | 395 | POSSIBLE NITRATE/NITRITE TRANSPORTER |
| Rv1738 | | 94 | CONSERVED HYPOTHETICAL PROTEIN |
| Rv1812c | | 400 | PROBABLE DEHYDROGENASE |
| Rv1813c | | 143 | CONSERVED HYPOTHETICAL PROTEIN |
| Rv1996 | | 317 | CONSERVED HYPOTHETICAL PROTEIN-USPA |
| Rv1997 | ctpF | 905 | PROBABLE METAL CATION TRANSPORTER P-TYPE ATPASE A |
| Rv1998 | | 258 | CONSERVED HYPOTHETICAL PROTEIN |
| Rv2003c | | 285 | CONSERVED HYPOTHETICAL PROTEIN |
| Rv2004c | | 498 | CONSERVED HYPOTHETICAL PROTEIN |
| Rv2005c | | 295 | CONSERVED HYPOTHETICAL PROTEIN-USPA |
| Rv2006 | otsB1 | 1327 | PROBABLE TREHALOSE-6-PHOSPHATE PHOSPHATASE |
| Rv2007c | fdxA | 114 | PROBABLE FERREDOXIN |
| Rv2028c | | 279 | CONSERVED HYPOTHETICAL PROTEIN-USPA |
| Rv2029c | pfkB | 339 | PROBABLE PHOSPHOHEXOKINASE |
| Rv2030c | | 681 | CONSERVED HYPOTHETICAL PROTEIN |
| Rv2031c | acr | 144 | HEAT SHOCK PROTEIN X (ALPHA-CRSTALLIN HOMOLOG) |
| Rv2032 | acg | 331 | CONSERVED HYPOTHETICAL PROTEIN |
| Rv2623 | TB31.7 | 297 | CONSERVED HYPOTHETICAL PROTEIN-USPA |
| Rv2624c | | 272 | CONSERVED HYPOTHETICAL PROTEIN-USPA |
| Rv2625c | | 393 | PROBABLE CONSERVED TRANSMEMBRANE PROTEIN |
| Rv2626c | | 143 | CONSERVED HYPOTHETICAL PROTEIN |
| Rv2627c | | 413 | CONSERVED HYPOTHETICAL PROTEIN |
| Rv2628 | | 120 | HYPOTHETICAL PROTEIN |
| Rv2629 | | 374 | CONSERVED HYPOTHETICAL PROTEIN |
| Rv2630 | | 179 | HYPOTHETICAL PROTEIN |
| Rv2631 | | 432 | CONSERVED HYPOTHETICAL PROTEIN |
| Rv3126c | | 104 | HYPOTHETICAL PROTEIN |
| Rv3127 | | 344 | CONSERVED HYPOTHETICAL PROTEIN |
| Rv3128c | | 337 | CONSERVED HYPOTHETICAL PROTEIN |
| Rv3129 | | 110 | CONSERVED HYPOTHETICAL PROTEIN |
| Rv3130c | | 463 | CONSERVED HYPOTHETICAL PROTEIN |
| Rv3131 | | 332 | CONSERVED HYPOTHETICAL PROTEIN |
| Rv3132c | | 578 | TWO COMPONENT SENSOR HISTIDINE KINASE |
| Rv3133c | dosR | 217 | TWO COMPONENT TRANSCRIPTIONAL REGULATORY PROTEIN |
| Rv3134c | | 268 | CONSERVED HYPOTHETICAL PROTEIN-USPA |

### Example 8

For pre-exposure (i.e. prophylactic) TB vaccines, it is generally accepted in the TB field that preclinical TB vaccine studies should include progressive screening and testing in relevant mouse, guinea pig and non-human primate models (Brandt et al. Infect. Immun. 2000; 68(2): 791-795; Olsen et al. Infect. Immun. 2004; 72(10):6148-50; Langermans et al. Vaccine 2005; 23(21):2740-50), including low-dose aerosol-infection challenge models (Williams et al. 2005, Tuberculosis (Edinb). 2005; 85(1-2):29-38). For post-exposure (i.e. therapeutic) TB vaccines, the existing animal models mimic human TB disease only partially (McMurray, Clin. Infect. Dis. 2000; 30 Suppl 3:S210-2) and extrapolation to humans is unclear. The present thinking in field of TB vaccine development is to boost immune response induced by BCG with a novel TB vaccine (e.g. McShane et al. Nat. Med. 2004; 10(11):1240-4; Orme Tuberculosis (Edinb). 2005; 85(1-2):13-7). However, boosting of immune responses can only be achieved if BCG has primed the responses in the first place.

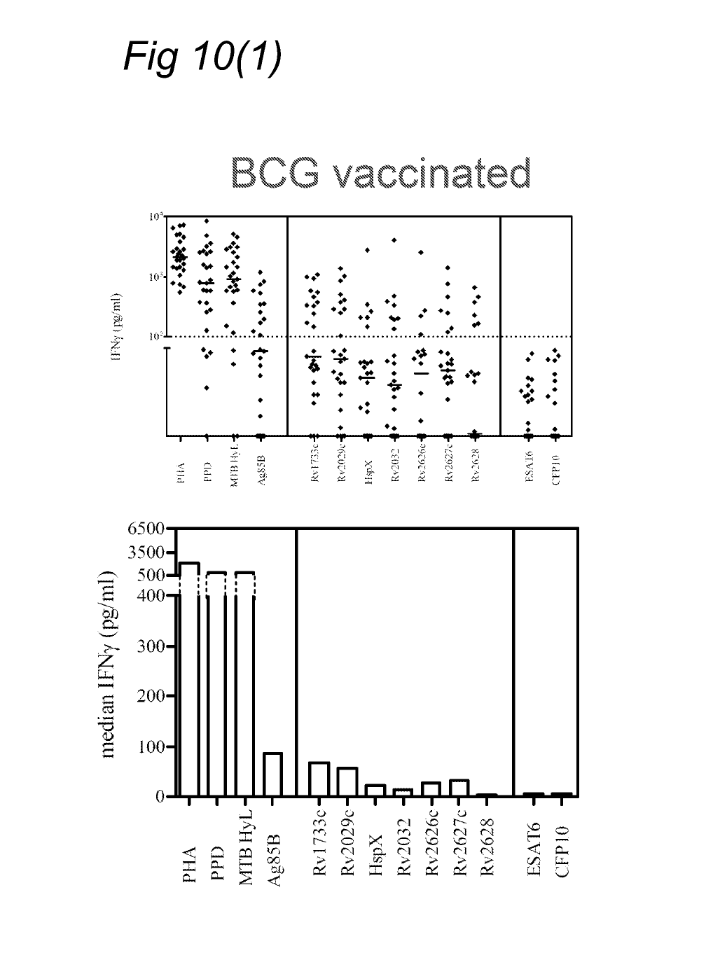

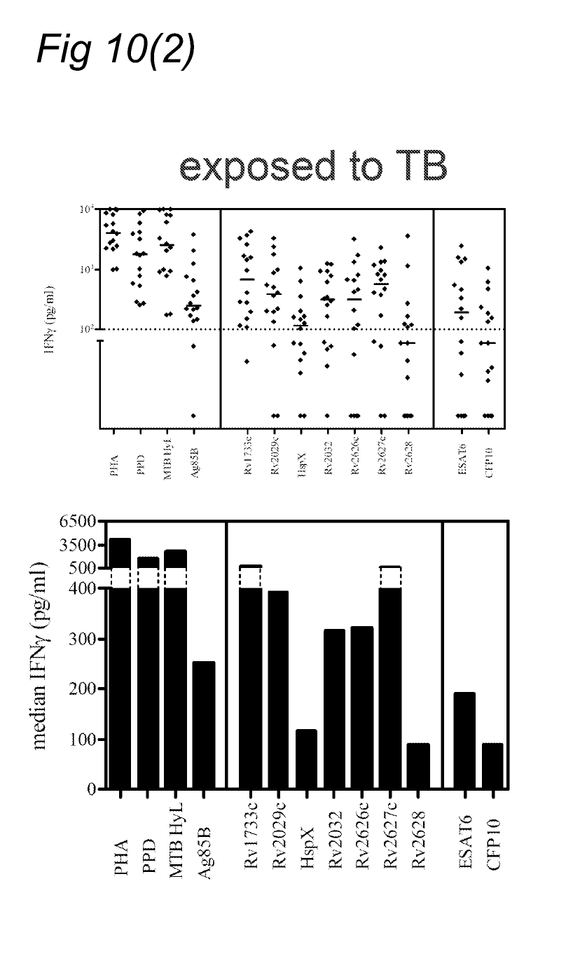

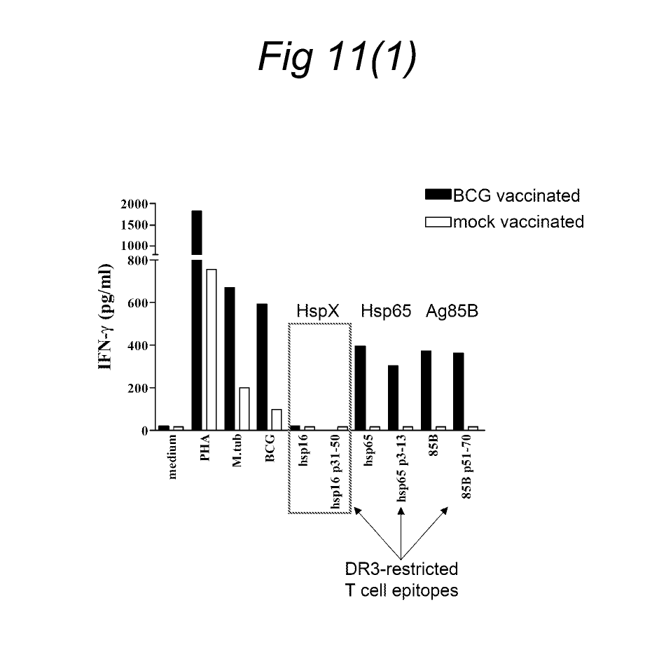

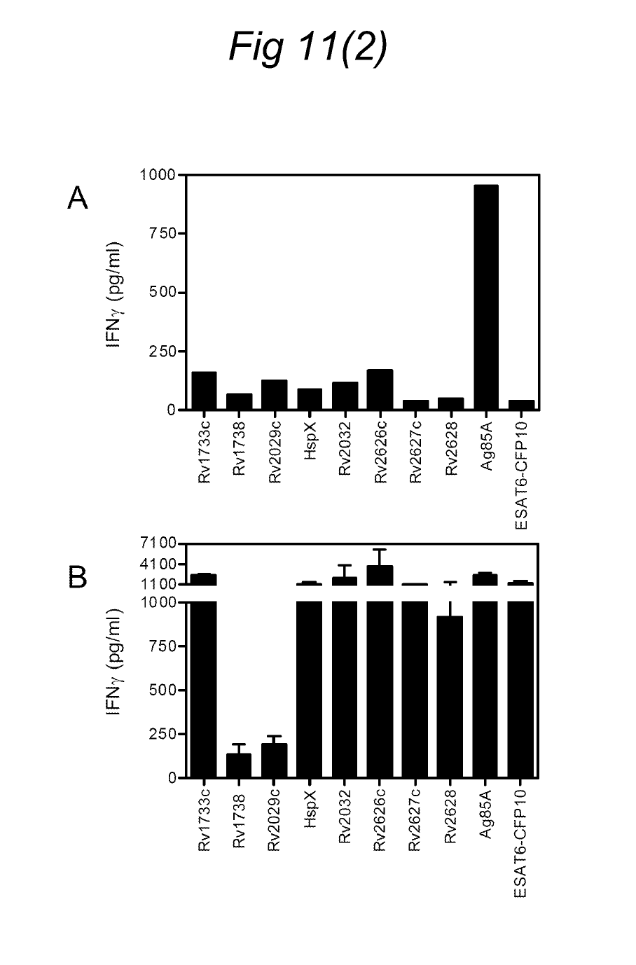

The TB vaccine presently in use is Mycobacterium bovis BCG. BCG protects young children against disseminated and severe forms of TB disease, but fails to protect against the most prevalent and contagious form of pulmonary TB in adults. Many hypotheses have been put forward to explain the failure of BCG, in particular that immune responses to BCG are influenced by exposure to environmental mycobacteria (Fine P E, 1995 Lancet. 346:1339-45). The current invention provides a better alternative. Conditions that BCG encounters during vaccination in the skin are different from the conditions which tubercle bacilli encounter during persistence in immune competent hosts, mainly in immune granulomas in the lungs. Consequently the antigen expression profiles will be different, including the corresponding immune recognition profiles. We found that BCG vaccination does not induce immune responses to TB latency antigens: When blood samples (PBMC) of BCG vaccinated healthy subjects are tested in vitro, very poor immune responses to TB latency antigens are observed, in comparison to individuals who have been exposed to TB (in vitro positive for responses to ESAT-6 and CFP10) (FIG. 10). The observation from this cross-sectional comparison was corroborated in (HLA-A2 and -DR3 transgenic) mice for HspX as well as for other TB latency antigens in ordinary inbred mice upon vaccination with BCG (FIG. 11).

Because immunodominant TB latency antigens are targeted as part of natural immunity against TB in humans, and not targeted by the current BCG vaccination procedures, they represent the most promising candidates for therapeutic TB vaccines. (Of note, none of the TB latency antigens are among the RD antigens, which are missing from BCG (Behr et al. 1999). In vitro expression profiling of BCG reveals that it is capable of expressing all TB latency antigens, except NarX/K2. Thus, although BCG in principle is capable of expressing TB latency antigens in vitro, apparently it does not encounter the proper conditions for the expression of the DosR regulon encoded antigens. The current invention provides DosR latency antigens which are more effective in vivo, selected from the group consisting of Rv1733c, Rv2029c (PfkB), Rv2627c, Rv2628, Rv0080, Rv1737c (NarK2), Rv1735c and Rv1736c (NarX), most preferably selected from Rv1733c, Rv2029c, Rv2627c and Rv0080, for use in compositions and/or vaccines, or alternatively for expression in and/or display on recombinant (BCG) mycobacteria. The invention also provides epitopes for T and B cells within these DosR antigens.

TABLE 3

| | | | | | |
|---|---|---|---|---|---|
| | | | Predicted | | |
| | | | epitope | | Combined |
| Supertype | ID | Pos. | sequence | Affinity | Score |

Predicted supertype HLA
class-I epitopes in Rv1733c

| | | | | | |
|---|---|---|---|---|---|
| A1 | Rv1733c | 67 | [symbol] | 0.30 | 2.30 |
| A1 | Rv1733c | 143 | [symbol] | 0.15 | 1.10 |
| A2 | Rv1733c | 170 | [symbol] | 0.41 | 1.10 |
| A2 | Rv1733c | 165 | [symbol] | 0.41 | 1.07 |
| A2 | Rv1733c | 143 | [symbol] | 0.38 | 1.03 |
| A2 | Rv1733c | 46 | [symbol] | 0.36 | 1.01 |
| A2 | Rv1733c | 181 | [symbol] | 0.39 | 0.97 |
| A2 | Rv1733c | 66 | [symbol] | 0.31 | 0.88 |
| A2 | Rv1733c | 111 | [symbol] | 0.26 | 0.76 |
| A3 | Rv1733c | 11 | [symbol] | 0.55 | 1.72 |
| A3 | Rv1733c | 23 | [symbol] | 0.44 | 1.42 |
| A3 | Rv1733c | 19 | [symbol] | 0.29 | 1.04 |
| A3 | Rv1733c | 127 | [symbol] | 0.24 | 0.82 |
| A3 | Rv1733c | 100 | [symbol] | 0.23 | 0.78 |
| A24 | Rv1733c | 197 | [symbol] | 0.65 | 1.05 |
| A24 | Rv1733c | 198 | [symbol] | 0.58 | 1.03 |
| A24 | Rv1733c | 46 | [symbol] | 0.63 | 1.02 |
| A24 | Rv1733c | 142 | [symbol] | 0.58 | 0.96 |
| A24 | Rv1733c | 51 | [symbol] | 0.47 | 0.87 |
| A24 | Rv1733c | 21 | [symbol] | 0.54 | 0.86 |
| A24 | Rv1733c | 161 | [symbol] | 0.51 | 0.84 |
| A24 | Rv1733c | 12 | [symbol] | 0.49 | 0.83 |
| A24 | Rv1733c | 47 | [symbol] | 0.52 | 0.83 |
| A24 | Rv1733c | 40 | [symbol] | 0.49 | 0.82 |
| A24 | Rv1733c | 8 | [symbol] | 0.43 | 0.78 |
| B7 | Rv1733c | 153 | [symbol] | 0.66 | 1.89 |
| B7 | Rv1733c | 59 | [symbol] | 0.59 | 1.70 |
| B7 | Rv1733c | 17 | [symbol] | 0.49 | 1.43 |
| B7 | Rv1733c | 177 | [symbol] | 0.46 | 1.39 |
| B7 | Rv1733c | 105 | [symbol] | 0.41 | 1.22 |
| B7 | Rv1733c | 175 | [symbol] | 0.38 | 1.19 |
| B7 | Rv1733c | 6 | [symbol] | 0.36 | 1.13 |
| B7 | Rv1733c | 174 | [symbol] | 0.32 | 0.99 |
| B7 | Rv1733c | 103 | [symbol] | 0.30 | 0.96 |
| B7 | Rv1733c | 80 | [symbol] | 0.34 | 0.92 |
| B8 | Rv1733c | 21 | [symbol] | 0.34 | 1.78 |
| B8 | Rv1733c | 114 | [symbol] | 0.34 | 0.92 |
| B27 | Rv1733c | 70 | [symbol] | 0.25 | 1.30 |
| B27 | Rv1733c | 26 | [symbol] | 0.19 | 1.09 |
| B27 | Rv1733c | 158 | [symbol] | 0.17 | 1.00 |
| B44 | Rv1733c | 8 | [symbol] | 0.20 | 1.56 |
| B44 | Rv1733c | 126 | [symbol] | 0.13 | 0.88 |
| B27 | Rv1737c | 322 | [symbol] | 0.17 | 0.96 |
| B27 | Rv1737c | 197 | [symbol] | 0.15 | 0.90 |
| B27 | Rv1737c | 271 | [symbol] | 0.13 | 0.80 |
| B44 | Rv1737c | 295 | [symbol] | 0.34 | 2.42 |
| B44 | Rv1737c | 209 | [symbol] | 0.16 | 1.13 |
| B58 | Rv1737c | 14 | [symbol] | 0.75 | 2.72 |
| B58 | Rv1737c | 207 | [symbol] | 0.62 | 2.40 |
| B58 | Rv1737c | 116 | [symbol] | 0.60 | 2.18 |
| B58 | Rv1737c | 290 | [symbol] | 0.48 | 1.83 |
| B58 | Rv1737c | 256 | [symbol] | 0.46 | 1.73 |
| B58 | Rv1737c | 60 | [symbol] | 0.37 | 1.41 |
| B58 | Rv1737c | 312 | [symbol] | 0.37 | 1.39 |
| B58 | Rv1737c | 39 | [symbol] | 0.35 | 1.32 |
| B58 | Rv1737c | 201 | [symbol] | 0.35 | 1.31 |
| B58 | Rv1737c | 133 | [symbol] | 0.27 | 0.95 |
| B58 | Rv1737c | 30 | [symbol] | 0.22 | 0.90 |
| B58 | Rv1737c | 211 | [symbol] | 0.18 | 0.80 |
| B58 | Rv1737c | 11 | [symbol] | 0.16 | 0.75 |
| B62 | Rv1737c | 24 | [symbol] | 0.55 | 1.66 |
| B62 | Rv1737c | 106 | [symbol] | 0.50 | 1.45 |
| B62 | Rv1737c | 89 | [symbol] | 0.45 | 1.39 |
| B62 | Rv1737c | 179 | [symbol] | 0.41 | 1.31 |
| B62 | Rv1737c | 217 | [symbol] | 0.42 | 1.31 |
| B62 | Rv1737c | 207 | [symbol] | 0.35 | 1.15 |
| B62 | Rv1737c | 124 | [symbol] | 0.37 | 1.13 |
| B62 | Rv1737c | 290 | [symbol] | 0.35 | 1.06 |
| B62 | Rv1737c | 112 | [symbol] | 0.32 | 1.04 |
| B62 | Rv1737c | 98 | [symbol] | 0.29 | 0.94 |
| B62 | Rv1737c | 229 | [symbol] | 0.27 | 0.91 |
| B62 | Rv1737c | 372 | [symbol] | 0.22 | 0.81 |
| B62 | Rv1737c | 367 | [symbol] | 0.26 | 0.81 |
| B62 | Rv1737c | 310 | [symbol] | 0.24 | 0.79 |
| B62 | Rv1737c | 141 | [symbol] | 0.26 | 0.78 |
| B62 | Rv1737c | 220 | [symbol] | 0.22 | 0.78 |
| B62 | Rv1737c | 95 | [symbol] | 0.24 | 0.76 |
| B62 | Rv1737c | 283 | [symbol] | 0.24 | 0.76 |

Predicted supertype HLA
class-I epitopes in Rv2029c

| | | | | | |
|---|---|---|---|---|---|
| A1 | Rv2029c | 319 | [symbol] | 0.52 | 3.69 |
| A1 | Rv2029c | 1 | [symbol] | 0.21 | 1.32 |
| A2 | Rv2029c | 117 | [symbol] | 0.54 | 1.41 |
| A2 | Rv2029c | 182 | [symbol] | 0.46 | 1.19 |
| A2 | Rv2029c | 296 | [symbol] | 0.44 | 1.07 |
| A2 | Rv2029c | 221 | [symbol] | 0.33 | 0.89 |
| A2 | Rv2029c | 82 | [symbol] | 0.26 | 0.83 |
| A2 | Rv2029c | 208 | [symbol] | 0.32 | 0.82 |
| A2 | Rv2029c | 60 | [symbol] | 0.28 | 0.80 |
| A2 | Rv2029c | 67 | [symbol] | 0.32 | 0.75 |
| A3 | Rv2029c | 104 | [symbol] | 0.66 | 2.07 |
| A3 | Rv2029c | 186 | [symbol] | 0.34 | 1.14 |
| A3 | Rv2029c | 101 | [symbol] | 0.25 | 0.92 |
| A3 | Rv2029c | 236 | [symbol] | 0.25 | 0.89 |
| A3 | Rv2029c | 275 | [symbol] | 0.25 | 0.88 |
| A3 | Rv2029c | 154 | [symbol] | 0.25 | 0.80 |
| A3 | Rv2029c | 269 | [symbol] | 0.22 | 0.80 |
| A24 | Rv2029c | 279 | [symbol] | 0.67 | 1.07 |
| A24 | Rv2029c | 143 | [symbol] | 0.65 | 1.05 |
| A24 | Rv2029c | 44 | [symbol] | 0.64 | 1.00 |
| A24 | Rv2029c | 158 | [symbol] | 0.62 | 0.98 |
| A24 | Rv2029c | 267 | [symbol] | 0.57 | 0.94 |
| A24 | Rv2029c | 115 | [symbol] | 0.55 | 0.93 |
| A24 | Rv2029c | 87 | [symbol] | 0.58 | 0.92 |
| A24 | Rv2029c | 192 | [symbol] | 0.52 | 0.85 |
| A24 | Rv2029c | 242 | [symbol] | 0.44 | 0.84 |
| A24 | Rv2029c | 226 | [symbol] | 0.50 | 0.83 |
| A24 | Rv2029c | 82 | [symbol] | 0.45 | 0.83 |
| A24 | Rv2029c | 108 | [symbol] | 0.41 | 0.79 |
| A24 | Rv2029c | 304 | [symbol] | 0.45 | 0.75 |
| B7 | Rv2029c | 11 | [symbol] | 0.66 | 1.93 |
| B7 | Rv2029c | 253 | [symbol] | 0.45 | 1.35 |
| B7 | Rv2029c | 115 | [symbol] | 0.34 | 1.12 |
| B7 | Rv2029c | 95 | [symbol] | 0.33 | 1.06 |
| B7 | Rv2029c | 136 | [symbol] | 0.31 | 1.05 |
| B7 | Rv2029c | 166 | [symbol] | 0.31 | 1.00 |
| B7 | Rv2029c | 226 | [symbol] | 0.29 | 0.97 |
| B7 | Rv2029c | 138 | [symbol] | 0.25 | 0.83 |
| B7 | Rv2029c | 224 | [symbol] | 0.25 | 0.79 |
| B7 | Rv2029c | 196 | [symbol] | 0.24 | 0.79 |
| B7 | Rv2029c | 250 | [symbol] | 0.23 | 0.75 |
| B8 | Rv2029c | 9 | [symbol] | 0.35 | 1.83 |
| B8 | Rv2029c | 192 | [symbol] | 0.31 | 1.63 |
| B8 | Rv2029c | 166 | [symbol] | 0.20 | 1.11 |
| B8 | Rv2029c | 110 | [symbol] | 0.19 | 1.07 |
| B8 | Rv2029c | 242 | [symbol] | 0.16 | 0.98 |
| B8 | Rv2029c | 222 | [symbol] | 0.14 | 0.76 |
| B27 | Rv2029c | 108 | [symbol] | 0.46 | 2.48 |
| B27 | Rv2029c | 168 | [symbol] | 0.28 | 1.57 |
| B27 | Rv2029c | 305 | [symbol] | 0.21 | 1.25 |
| B27 | Rv2029c | 225 | [symbol] | 0.16 | 0.76 |
| B27 | Rv2029c | 161 | [symbol] | 0.12 | 0.75 |
| B44 | Rv2029c | 106 | [symbol] | 0.15 | 1.30 |
| B44 | Rv2029c | 313 | [symbol] | 0.18 | 1.13 |
| B44 | Rv2029c | 100 | [symbol] | 0.16 | 0.98 |
| B44 | Rv2029c | 227 | [symbol] | 0.14 | 0.91 |
| B44 | Rv2029c | 201 | [symbol] | 0.11 | 0.88 |
| B44 | Rv2029c | 320 | [symbol] | 0.10 | 0.77 |
| B58 | Rv2029c | 271 | [symbol] | 0.74 | 2.62 |
| B58 | Rv2029c | 321 | [symbol] | 0.50 | 1.81 |
| B58 | Rv2029c | 109 | [symbol] | 0.25 | 0.90 |
| B58 | Rv2029c | 15 | [symbol] | 0.23 | 0.89 |
| B58 | Rv2029c | 242 | [symbol] | 0.18 | 0.87 |
| B58 | Rv2029c | 232 | [symbol] | 0.19 | 0.79 |
| B62 | Rv2029c | 183 | [symbol] | 0.57 | 1.61 |
| B62 | Rv2029c | 82 | [symbol] | 0.51 | 1.52 |
| B62 | Rv2029c | 240 | [symbol] | 0.34 | 0.94 |
| B62 | Rv2029c | 37 | [symbol] | 0.24 | 0.86 |
| B62 | Rv2029c | 61 | [symbol] | 0.26 | 0.84 |
| B62 | Rv2029c | 136 | [symbol] | 0.25 | 0.83 |
| B62 | Rv2029c | 60 | [symbol] | 0.24 | 0.75 |

Predicted supertype HLA
class-I epitopes in Rv2627c

| | | | | | |
|---|---|---|---|---|---|
| A1 | Rv2627c | 35 | [symbol] | 0.16 | 1.28 |
| A1 | Rv2627c | 127 | [symbol] | 0.13 | 1.14 |
| A1 | Rv2627c | 157 | [symbol] | 0.09 | 0.90 |
| A1 | Rv2627c | 92 | [symbol] | 0.09 | 0.85 |
| A1 | Rv2627c | 2 | [symbol] | 0.11 | 0.77 |
| A2 | Rv2627c | 29 | [symbol] | 0.51 | 1.29 |
| A2 | Rv2627c | 271 | [symbol] | 0.46 | 1.25 |
| A2 | Rv2627c | 320 | [symbol] | 0.40 | 1.10 |
| A2 | Rv2627c | 275 | [symbol] | 0.35 | 0.98 |
| A2 | Rv2627c | 43 | [symbol] | 0.34 | 0.94 |
| A2 | Rv2627c | 286 | [symbol] | 0.36 | 0.94 |
| A2 | Rv2627c | 282 | [symbol] | 0.37 | 0.89 |
| A2 | Rv2627c | 245 | [symbol] | 0.26 | 0.78 |
| A3 | Rv2627c | 398 | [symbol] | 0.42 | 1.35 |
| A3 | Rv2627c | 8 | [symbol] | 0.39 | 1.27 |
| A3 | Rv2627c | 212 | [symbol] | 0.37 | 1.27 |
| A3 | Rv2627c | 243 | [symbol] | 0.38 | 1.22 |
| A3 | Rv2627c | 312 | [symbol] | 0.37 | 1.21 |
| A3 | Rv2627c | 192 | [symbol] | 0.36 | 1.14 |
| A3 | Rv2627c | 69 | [symbol] | 0.32 | 1.05 |
| A3 | Rv2627c | 92 | [symbol] | 0.26 | 0.98 |
| A3 | Rv2627c | 100 | [symbol] | 0.28 | 0.91 |
| A3 | Rv2627c | 194 | [symbol] | 0.30 | 0.90 |
| A3 | Rv2627c | 33 | [symbol] | 0.26 | 0.88 |
| A3 | Rv2627c | 189 | [symbol] | 0.24 | 0.87 |
| A24 | Rv2627c | 374 | [symbol] | 0.87 | 1.43 |
| A24 | Rv2627c | 345 | [symbol] | 0.80 | 1.24 |
| A24 | Rv2627c | 157 | [symbol] | 0.70 | 1.22 |
| A24 | Rv2627c | 104 | [symbol] | 0.70 | 1.21 |
| A24 | Rv2627c | 64 | [symbol] | 0.65 | 1.05 |
| A24 | Rv2627c | 135 | [symbol] | 0.58 | 1.02 |
| A24 | Rv2627c | 105 | [symbol] | 0.58 | 1.02 |
| A24 | Rv2627c | 188 | [symbol] | 0.55 | 1.00 |
| A24 | Rv2627c | 383 | [symbol] | 0.61 | 0.98 |
| A24 | Rv2627c | 329 | [symbol] | 0.60 | 0.95 |
| A24 | Rv2627c | 291 | [symbol] | 0.59 | 0.95 |
| A24 | Rv2627c | 98 | [symbol] | 0.58 | 0.92 |
| A24 | Rv2627c | 199 | [symbol] | 0.57 | 0.90 |
| A24 | Rv2627c | 264 | [symbol] | 0.56 | 0.89 |
| A24 | Rv2627c | 131 | [symbol] | 0.45 | 0.86 |
| A24 | Rv2627c | 130 | [symbol] | 0.46 | 0.86 |
| A24 | Rv2627c | 216 | [symbol] | 0.58 | 0.85 |
| A24 | Rv2627c | 26 | [symbol] | 0.52 | 0.85 |
| A24 | Rv2627c | 193 | [symbol] | 0.51 | 0.85 |
| A24 | Rv2627c | 14 | [symbol] | 0.49 | 0.84 |
| A24 | Rv2627c | 111 | [symbol] | 0.52 | 0.83 |
| A24 | Rv2627c | 275 | [symbol] | 0.49 | 0.82 |
| A24 | Rv2627c | 170 | [symbol] | 0.48 | 0.81 |
| A24 | Rv2627c | 324 | [symbol] | 0.49 | 0.79 |
| A24 | Rv2627c | 57 | [symbol] | 0.48 | 0.78 |
| A24 | Rv2627c | 89 | [symbol] | 0.46 | 0.78 |
| A24 | Rv2627c | 21 | [symbol] | 0.44 | 0.76 |
| A24 | Rv2627c | 366 | [symbol] | 0.46 | 0.76 |
| B7 | Rv2627c | 355 | [symbol] | 0.63 | 1.87 |
| B7 | Rv2627c | 78 | [symbol] | 0.57 | 1.82 |
| B7 | Rv2627c | 313 | [symbol] | 0.53 | 1.56 |
| B7 | Rv2627c | 18 | [symbol] | 0.48 | 1.44 |
| B7 | Rv2627c | 55 | [symbol] | 0.42 | 1.22 |
| B7 | Rv2627c | 170 | [symbol] | 0.36 | 1.17 |
| B7 | Rv2627c | 404 | [symbol] | 0.44 | 1.13 |
| B7 | Rv2627c | 388 | [symbol] | 0.34 | 1.09 |
| B7 | Rv2627c | 338 | [symbol] | 0.29 | 1.04 |
| B7 | Rv2627c | 386 | [symbol] | 0.34 | 1.03 |
| B7 | Rv2627c | 50 | [symbol] | 0.32 | 1.00 |
| B7 | Rv2627c | 393 | [symbol] | 0.31 | 0.97 |
| B7 | Rv2627c | 126 | [symbol] | 0.29 | 0.95 |
| B7 | Rv2627c | 173 | [symbol] | 0.29 | 0.90 |
| B7 | Rv2627c | 275 | [symbol] | 0.26 | 0.88 |
| B7 | Rv2627c | 188 | [symbol] | 0.23 | 0.87 |
| B7 | Rv2627c | 294 | [symbol] | 0.26 | 0.85 |
| B7 | Rv2627c | 36 | [symbol] | 0.25 | 0.82 |
| B7 | Rv2627c | 244 | [symbol] | 0.22 | 0.76 |
| B7 | Rv2627c | 197 | [symbol] | 0.21 | 0.75 |
| B8 | Rv2627c | 355 | [symbol] | 0.25 | 1.34 |
| B8 | Rv2627c | 367 | [symbol] | 0.22 | 1.28 |
| B8 | Rv2627c | 173 | [symbol] | 0.24 | 1.26 |
| B8 | Rv2627c | 64 | [symbol] | 0.22 | 1.24 |
| B8 | Rv2627c | 126 | [symbol] | 0.21 | 1.17 |
| B8 | Rv2627c | 248 | [symbol] | 0.19 | 0.95 |
| B8 | Rv2627c | 130 | [symbol] | 0.15 | 0.93 |
| B8 | Rv2627c | 14 | [symbol] | 0.15 | 0.88 |
| B8 | Rv2627c | 161 | [symbol] | 0.14 | 0.85 |
| B8 | Rv2627c | 387 | [symbol] | 0.17 | 0.83 |
| B8 | Rv2627c | 388 | [symbol] | 0.14 | 0.83 |
| B8 | Rv2627c | 275 | [symbol] | 0.14 | 0.81 |
| B8 | Rv2627c | 105 | [symbol] | 0.12 | 0.78 |
| B8 | Rv2627c | 89 | [symbol] | 0.13 | 0.78 |
| B8 | Rv2627c | 341 | [symbol] | 0.14 | 0.76 |
| B27 | Rv2627c | 131 | [symbol] | 0.51 | 2.75 |
| B27 | Rv2627c | 130 | [symbol] | 0.46 | 2.50 |
| B27 | Rv2627c | 304 | [symbol] | 0.42 | 2.21 |
| B27 | Rv2627c | 76 | [symbol] | 0.31 | 1.74 |
| B27 | Rv2627c | 326 | [symbol] | 0.31 | 1.70 |
| B27 | Rv2627c | 389 | [symbol] | 0.34 | 1.61 |
| B27 | Rv2627c | 388 | [symbol] | 0.29 | 1.60 |
| B27 | Rv2627c | 250 | [symbol] | 0.29 | 1.35 |
| B27 | Rv2627c | 315 | [symbol] | 0.27 | 1.25 |
| B27 | Rv2627c | 135 | [symbol] | 0.17 | 1.06 |
| B27 | Rv2627c | 103 | [symbol] | 0.17 | 0.88 |
| B27 | Rv2627c | 361 | [symbol] | 0.15 | 0.79 |
| B27 | Rv2627c | 187 | [symbol] | 0.14 | 0.79 |
| B44 | Rv2627c | 91 | [symbol] | 0.29 | 2.22 |
| B44 | Rv2627c | 97 | [symbol] | 0.26 | 1.89 |
| B44 | Rv2627c | 284 | [symbol] | 0.16 | 1.20 |
| B44 | Rv2627c | 259 | [symbol] | 0.14 | 1.11 |
| B44 | Rv2627c | 364 | [symbol] | 0.14 | 1.10 |
| B44 | Rv2627c | 86 | [symbol] | 0.13 | 0.91 |
| B44 | Rv2627c | 148 | [symbol] | 0.12 | 0.88 |
| B44 | Rv2627c | 67 | [symbol] | 0.11 | 0.86 |
| B58 | Rv2627c | 32 | [symbol] | 0.39 | 1.42 |
| B58 | Rv2627c | 147 | [symbol] | 0.36 | 1.34 |
| B58 | Rv2627c | 239 | [symbol] | 0.29 | 1.08 |
| B58 | Rv2627c | 366 | [symbol] | 0.26 | 1.02 |
| B58 | Rv2627c | 89 | [symbol] | 0.23 | 0.94 |
| B58 | Rv2627c | 241 | [symbol] | 0.25 | 0.92 |
| B58 | Rv2627c | 97 | [symbol] | 0.21 | 0.86 |
| B58 | Rv2627c | 359 | [symbol] | 0.22 | 0.82 |
| B58 | Rv2627c | 169 | [symbol] | 0.20 | 0.80 |
| B58 | Rv2627c | 247 | [symbol] | 0.18 | 0.75 |
| B62 | Rv2627c | 35 | [symbol] | 0.50 | 1.49 |
| B62 | Rv2627c | 105 | [symbol] | 0.47 | 1.41 |
| B62 | Rv2627c | 126 | [symbol] | 0.42 | 1.24 |
| B62 | Rv2627c | 222 | [symbol] | 0.42 | 1.22 |
| B62 | Rv2627c | 383 | [symbol] | 0.38 | 1.10 |
| B62 | Rv2627c | 367 | [symbol] | 0.31 | 1.02 |
| B62 | Rv2627c | 21 | [symbol] | 0.31 | 0.97 |
| B62 | Rv2627c | 267 | [symbol] | 0.28 | 0.87 |
| B62 | Rv2627c | 57 | [symbol] | 0.28 | 0.85 |
| B62 | Rv2627c | 135 | [symbol] | 0.23 | 0.83 |
| B62 | Rv2627c | 45 | [symbol] | 0.28 | 0.82 |
| B62 | Rv2627c | 374 | [symbol] | 0.21 | 0.78 |

Predicted supertype HLA
class-I epitopes in Rv2628

| | | | | | |
|---|---|---|---|---|---|
| A1 | Rv2628 | 55 | [symbol] | 0.10 | 0.77 |
| A1 | Rv2628 | 98 | [symbol] | 0.07 | 0.76 |
| A2 | Rv2628 | 88 | [symbol] | 0.38 | 0.95 |
| A2 | Rv2628 | 79 | [symbol] | 0.35 | 0.75 |
| A3 | Rv2628 | 58 | [symbol] | 0.47 | 1.57 |
| A3 | Rv2628 | 98 | [symbol] | 0.32 | 1.21 |
| A3 | Rv2628 | 35 | [symbol] | 0.40 | 1.19 |
| A3 | Rv2628 | 50 | [symbol] | 0.32 | 1.11 |
| A3 | Rv2628 | 55 | [symbol] | 0.27 | 0.88 |
| A3 | Rv2628 | 38 | [symbol] | 0.25 | 0.84 |
| A24 | Rv2628 | 31 | [symbol] | 0.61 | 0.99 |
| B7 | Rv2628 | 5 | [symbol] | 0.70 | 1.95 |
| B7 | Rv2628 | 43 | [symbol] | 0.63 | 1.70 |
| B7 | Rv2628 | 64 | [symbol] | 0.31 | 0.89 |
| B8 | Rv2628 | 31 | [symbol] | 0.17 | 0.95 |
| B8 | Rv2628 | 44 | [symbol] | 0.16 | 0.88 |
| B27 | Rv2628 | 24 | [symbol] | 0.43 | 2.13 |
| B27 | Rv2628 | 27 | [symbol] | 0.39 | 2.02 |
| B27 | Rv2628 | 45 | [symbol] | 0.29 | 1.67 |
| B44 | Rv2628 | 109 | [symbol] | 0.17 | 1.19 |
| B44 | Rv2628 | 81 | [symbol] | 0.10 | 0.82 |
| B58 | Rv2628 | 72 | [symbol] | 0.42 | 1.60 |
| B58 | Rv2628 | 11 | [symbol] | 0.38 | 1.46 |
| B62 | Rv2628 | 98 | [symbol] | 0.45 | 1.40 |
| B62 | Rv2628 | 3 | [symbol] | 0.39 | 1.11 |
| B62 | Rv2628 | 45 | [symbol] | 0.26 | 0.91 |

Predicted supertype HLA
class-I epitopes in Rv0080c

| | | | | | |
|---|---|---|---|---|---|
| A2 | Rv0080c | 120 | [symbol] | 0.45 | 1.11 |
| A2 | Rv0080c | 45 | [symbol] | 0.35 | 0.96 |
| A2 | Rv0080c | 100 | [symbol] | 0.30 | 0.85 |
| A3 | Rv0080c | 53 | [symbol] | 0.33 | 1.06 |
| A3 | Rv0080c | 108 | [symbol] | 0.23 | 0.78 |
| A24 | Rv0080c | 96 | [symbol] | 0.77 | 1.20 |
| A24 | Rv0080c | 116 | [symbol] | 0.80 | 1.17 |
| A24 | Rv0080c | 30 | [symbol] | 0.40 | 0.78 |
| B7 | Rv0080c | 35 | [symbol] | 0.61 | 1.79 |
| B7 | Rv0080c | 38 | [symbol] | 0.49 | 1.39 |
| B7 | Rv0080c | 9 | [symbol] | 0.46 | 1.39 |
| B7 | Rv0080c | 2 | [symbol] | 0.30 | 0.83 |
| B7 | Rv0080c | 121 | [symbol] | 0.23 | 0.76 |
| B8 | Rv0080c | 38 | [symbol] | 0.21 | 1.03 |
| B8 | Rv0080c | 19 | [symbol] | 0.16 | 0.88 |
| B27 | Rv0080 | 6 | [symbol] | 0.54 | 2.79 |
| B27 | Rv0080 | 56 | [symbol] | 0.46 | 2.42 |
| B27 | Rv0080 | 92 | [symbol] | 0.43 | 2.25 |
| B27 | Rv0080 | 93 | [symbol] | 0.31 | 1.65 |
| B27 | Rv0080 | 115 | [symbol] | 0.16 | 0.80 |
| B44 | Rv0080 | 14 | [symbol] | 0.15 | 0.98 |
| B44 | Rv0080 | 137 | [symbol] | 0.12 | 0.88 |
| B58 | Rv0080 | 74 | [symbol] | 0.29 | 1.11 |

Predidcted supertype HLA
class-I epitopes in Rv1735c

| | | | | | |
|---|---|---|---|---|---|
| A1 | Rv1735c | 57 | z,899 | 0.08 | 0.83 |
| A2 | Rv1735c | 117 | [symbol] | 0.44 | 1.19 |
| A2 | Rv1735c | 68 | [symbol] | 0.42 | 1.18 |
| A2 | Rv1735c | 43 | [symbol] | 0.46 | 1.15 |
| A2 | Rv1735c | 16 | [symbol] | 0.40 | 1.07 |
| A2 | Rv1735c | 108 | [symbol] | 0.35 | 0.93 |
| A2 | Rv1735c | 87 | [symbol] | 0.35 | 0.93 |
| A2 | Rv1735c | 77 | [symbol] | 0.29 | 0.87 |
| A2 | Rv1735c | 7 | [symbol] | 0.29 | 0.81 |
| A2 | Rv1735c | 28 | [symbol] | 0.29 | 0.80 |
| A2 | Rv1735c | 104 | [symbol] | 0.31 | 0.80 |
| A2 | Rv1735c | 136 | [symbol] | 0.28 | 0.79 |
| A2 | Rv1735c | 106 | [symbol] | 0.22 | 0.76 |
| A3 | Rv1735c | 57 | [symbol] | 0.25 | 0.99 |
| A3 | Rv1735c | 112 | [symbol] | 0.34 | 0.98 |
| A3 | Rv1735c | 77 | [symbol] | 0.27 | 0.98 |
| A3 | Rv1735c | 71 | [symbol] | 0.21 | 0.88 |
| A3 | Rv1735c | 35 | [symbol] | 0.24 | 0.86 |
| A3 | Rv1735c | 47 | [symbol] | 0.26 | 0.85 |
| A24 | Rv1735c | 78 | [symbol] | 0.84 | 1.30 |
| A24 | Rv1735c | 151 | [symbol] | 0.78 | 1.29 |
| A24 | Rv1735c | 32 | [symbol] | 0.71 | 1.19 |
| A24 | Rv1735c | 113 | [symbol] | 0.68 | 1.09 |
| A24 | Rv1735c | 110 | [symbol] | 0.67 | 1.04 |
| A24 | Rv1735c | 64 | [symbol] | 0.67 | 1.03 |
| A24 | Rv1735c | 21 | [symbol] | 0.66 | 1.02 |
| A24 | Rv1735c | 109 | [symbol] | 0.63 | 0.97 |
| A24 | Rv1735c | 104 | [symbol] | 0.66 | 0.96 |
| A24 | Rv1735c | 106 | [symbol] | 0.52 | 0.95 |
| A24 | Rv1735c | 37 | [symbol] | 0.65 | 0.93 |
| A24 | Rv1735c | 93 | [symbol] | 0.52 | 0.92 |
| A24 | Rv1735c | 29 | [symbol] | 0.48 | 0.90 |
| A24 | Rv1735c | 90 | [symbol] | 0.54 | 0.88 |
| A24 | Rv1735c | 36 | [symbol] | 0.55 | 0.86 |
| A24 | Rv1735c | 144 | [symbol] | 0.51 | 0.85 |
| A24 | Rv1735c | 73 | [symbol] | 0.53 | 0.85 |
| A24 | Rv1735c | 53 | [symbol] | 0.49 | 0.80 |
| A24 | Rv1735c | 80 | [symbol] | 0.46 | 0.75 |
| B7 | Rv1735c | 45 | [symbol] | 0.63 | 1.84 |
| B7 | Rv1735c | 61 | [symbol] | 0.39 | 1.23 |
| B7 | Rv1735c | 104 | [symbol] | 0.40 | 1.16 |
| B7 | Rv1735c | 157 | [symbol] | 0.34 | 1.09 |
| B7 | Rv1735c | 60 | [symbol] | 0.29 | 0.78 |
| B7 | Rv1735c | 115 | [symbol] | 0.28 | 0.77 |
| B8 | Rv1735c | 53 | [symbol] | 0.26 | 1.37 |
| B8 | Rv1735c | 110 | [symbol] | 0.13 | 0.75 |
| B27 | Rv1735c | 84 | [symbol] | 0.28 | 1.56 |
| B44 | Rv1735c | 99 | [symbol] | 0.12 | 0.95 |
| B44 | Rv1735c | 15 | [symbol] | 0.13 | 0.89 |
| B44 | Rv1735c | 101 | [symbol] | 0.13 | 0.87 |
| B44 | Rv1735c | 65 | [symbol] | 0.11 | 0.79 |
| B58 | Rv1735c | 30 | [symbol] | 0.59 | 2.16 |
| B58 | Rv1735c | 96 | [symbol] | 0.59 | 2.09 |
| B58 | Rv1735c | 35 | [symbol] | 0.31 | 1.24 |
| B58 | Rv1735c | 102 | [symbol] | 0.31 | 1.12 |
| B58 | Rv1735c | 112 | [symbol] | 0.24 | 0.82 |
| B58 | Rv1735c | 157 | [symbol] | 0.19 | 0.80 |
| B58 | Rv1735c | 127 | [symbol] | 0.19 | 0.77 |
| B62 | Rv1735c | 29 | [symbol] | 0.43 | 1.32 |
| B62 | Rv1735c | 106 | [symbol] | 0.39 | 1.24 |
| B62 | Rv1735c | 76 | [symbol] | 0.31 | 1.01 |
| B62 | Rv1735c | 71 | [symbol] | 0.29 | 1.00 |
| B62 | Rv1735c | 144 | [symbol] | 0.28 | 0.87 |
| B62 | Rv1735c | 43 | [symbol] | 0.28 | 0.76 |

Predicted supertype HLA class-I
epitopes in Rv1736c (NarX)

| | | | | | |
|---|---|---|---|---|---|
| A1 | Rv1736c | 448 | [symbol] | 0.53 | 3.83 |
| A1 | Rv1736c | 591 | [symbol] | 0.23 | 1.81 |
| A1 | Rv1736c | 260 | [symbol] | 0.18 | 1.45 |
| A1 | Rv1736c | 224 | [symbol] | 0.20 | 1.40 |
| A1 | Rv1736c | 223 | [symbol] | 0.17 | 1.37 |
| A1 | Rv1736c | 561 | [symbol] | 0.17 | 1.35 |
| A1 | Rv1736c | 536 | [symbol] | 0.17 | 1.35 |
| A1 | Rv1736c | 622 | [symbol] | 0.16 | 1.21 |
| A1 | Rv1736c | 516 | [symbol] | 0.11 | 1.02 |
| A1 | Rv1736c | 442 | [symbol] | 0.14 | 1.00 |
| A1 | Rv1736c | 620 | [symbol] | 0.09 | 0.89 |
| A1 | Rv1736c | 106 | [symbol] | 0.08 | 0.82 |
| A1 | Rv1736c | 395 | [symbol] | 0.08 | 0.77 |
| A2 | Rv1736c | 416 | [symbol] | 0.61 | 1.56 |
| A2 | Rv1736c | 494 | [symbol] | 0.58 | 1.49 |
| A2 | Rv1736c | 611 | [symbol] | 0.56 | 1.46 |
| A2 | Rv1736c | 365 | [symbol] | 0.43 | 1.15 |
| A2 | Rv1736c | 596 | [symbol] | 0.40 | 1.07 |
| A2 | Rv1736c | 505 | [symbol] | 0.42 | 1.05 |
| A2 | Rv1736c | 204 | [symbol] | 0.41 | 1.04 |
| A2 | Rv1736c | 381 | [symbol] | 0.38 | 1.03 |
| A2 | Rv1736c | 466 | [symbol] | 0.38 | 1.01 |
| A2 | Rv1736c | 515 | [symbol] | 0.36 | 1.00 |
| A2 | Rv1736c | 427 | [symbol] | 0.38 | 0.99 |
| A2 | Rv1736c | 415 | [symbol] | 0.43 | 0.99 |
| A2 | Rv1736c | 603 | [symbol] | 0.41 | 0.95 |
| A2 | Rv1736c | 399 | [symbol] | 0.34 | 0.94 |
| A2 | Rv1736c | 226 | [symbol] | 0.32 | 0.93 |
| A2 | Rv1736c | 304 | [symbol] | 0.35 | 0.92 |
| A2 | Rv1736c | 322 | [symbol] | 0.32 | 0.86 |
| A2 | Rv1736c | 54 | [symbol] | 0.30 | 0.85 |
| A2 | Rv1736c | 362 | [symbol] | 0.34 | 0.83 |
| A2 | Rv1736c | 544 | [symbol] | 0.29 | 0.82 |
| A2 | Rv1736c | 420 | [symbol] | 0.28 | 0.79 |
| A2 | Rv1736c | 283 | [symbol] | 0.26 | 0.78 |
| A2 | Rv1736c | 591 | [symbol] | 0.22 | 0.78 |
| A2 | Rv1736c | 391 | [symbol] | 0.29 | 0.78 |
| A3 | Rv1736c | 454 | [symbol] | 0.51 | 1.65 |
| A3 | Rv1736c | 628 | [symbol] | 0.41 | 1.48 |
| A3 | Rv1736c | 623 | [symbol] | 0.44 | 1.37 |
| A3 | Rv1736c | 128 | [symbol] | 0.42 | 1.36 |
| A3 | Rv1736c | 434 | [symbol] | 0.41 | 1.31 |
| A3 | Rv1736c | 564 | [symbol] | 0.40 | 1.30 |
| A3 | Rv1736c | 90 | [symbol] | 0.32 | 1.19 |
| A3 | Rv1736c | 625 | [symbol] | 0.34 | 1.16 |
| A3 | Rv1736c | 631 | [symbol] | 0.33 | 1.14 |
| A3 | Rv1736c | 432 | [symbol] | 0.35 | 1.13 |
| A3 | Rv1736c | 519 | [symbol] | 0.34 | 1.10 |
| A3 | Rv1736c | 580 | [symbol] | 0.33 | 1.08 |
| A3 | Rv1736c | 419 | [symbol] | 0.28 | 1.08 |
| A3 | Rv1736c | 2 | [symbol] | 0.27 | 0.95 |
| A3 | Rv1736c | 395 | [symbol] | 0.23 | 0.95 |
| A3 | Rv1736c | 32 | [symbol] | 0.29 | 0.95 |
| A3 | Rv1736c | 372 | [symbol] | 0.25 | 0.91 |
| A3 | Rv1736c | 112 | [symbol] | 0.26 | 0.90 |
| A3 | Rv1736c | 275 | [symbol] | 0.31 | 0.89 |
| A3 | Rv1736c | 518 | [symbol] | 0.24 | 0.88 |
| A3 | Rv1736c | 536 | [symbol] | 0.23 | 0.88 |
| A3 | Rv1736c | 451 | [symbol] | 0.26 | 0.87 |
| A3 | Rv1736c | 157 | [symbol] | 0.26 | 0.83 |
| A3 | Rv1736c | 371 | [symbol] | 0.21 | 0.80 |
| A3 | Rv1736c | 310 | [symbol] | 0.21 | 0.77 |
| A3 | Rv1736c | 9 | [symbol] | 0.21 | 0.76 |
| A24 | Rv1736c | 384 | [symbol] | 0.84 | 1.30 |
| A24 | Rv1736c | 499 | [symbol] | 0.82 | 1.28 |
| A24 | Rv1736c | 131 | [symbol] | 0.82 | 1.27 |
| A24 | Rv1736c | 438 | [symbol] | 0.82 | 1.27 |
| A24 | Rv1736c | 361 | [symbol] | 0.76 | 1.26 |
| A24 | Rv1736c | 614 | [symbol] | 0.79 | 1.23 |
| A24 | Rv1736c | 597 | [symbol] | 0.79 | 1.22 |
| A24 | Rv1736c | 543 | [symbol] | 0.81 | 1.22 |
| A24 | Rv1736c | 226 | [symbol] | 0.76 | 1.21 |
| A24 | Rv1736c | 627 | [symbol] | 0.77 | 1.19 |
| A24 | Rv1736c | 570 | [symbol] | 0.74 | 1.17 |
| A24 | Rv1736c | 455 | [symbol] | 0.76 | 1.17 |
| A24 | Rv1736c | 258 | [symbol] | 0.76 | 1.16 |
| A24 | Rv1736c | 447 | [symbol] | 0.71 | 1.11 |
| A24 | Rv1736c | 607 | [symbol] | 0.71 | 1.10 |
| A24 | Rv1736c | 440 | [symbol] | 0.72 | 1.06 |
| A24 | Rv1736c | 602 | [symbol] | 0.60 | 1.06 |
| A24 | Rv1736c | 621 | [symbol] | 0.67 | 1.05 |
| A24 | Rv1736c | 577 | [symbol] | 0.63 | 1.03 |
| A24 | Rv1736c | 402 | [symbol] | 0.64 | 0.98 |
| A24 | Rv1736c | 610 | [symbol] | 0.61 | 0.98 |
| A24 | Rv1736c | 583 | [symbol] | 0.60 | 0.95 |
| A24 | Rv1736c | 571 | [symbol] | 0.54 | 0.95 |
| A24 | Rv1736c | 111 | [symbol] | 0.60 | 0.94 |
| A24 | Rv1736c | 191 | [symbol] | 0.58 | 0.94 |
| A24 | Rv1736c | 219 | [symbol] | 0.53 | 0.93 |
| A24 | Rv1736c | 601 | [symbol] | 0.58 | 0.93 |
| A24 | Rv1736c | 604 | [symbol] | 0.63 | 0.93 |
| A24 | Rv1736c | 599 | [symbol] | 0.56 | 0.91 |
| A24 | Rv1736c | 222 | [symbol] | 0.56 | 0.90 |
| A24 | Rv1736c | 460 | [symbol] | 0.50 | 0.90 |
| A24 | Rv1736c | 266 | [symbol] | 0.54 | 0.89 |
| A24 | Rv1736c | 547 | [symbol] | 0.54 | 0.89 |
| A24 | Rv1736c | 291 | [symbol] | 0.53 | 0.86 |
| A24 | Rv1736c | 596 | [symbol] | 0.53 | 0.84 |
| A24 | Rv1736c | 634 | [symbol] | 0.50 | 0.84 |
| A24 | Rv1736c | 622 | [symbol] | 0.51 | 0.83 |
| A24 | Rv1736c | 467 | [symbol] | 0.51 | 0.82 |
| A24 | Rv1736c | 635 | [symbol] | 0.54 | 0.82 |
| A24 | Rv1736c | 120 | [symbol] | 0.54 | 0.81 |
| A24 | Rv1736c | 13 | [symbol] | 0.44 | 0.80 |
| A24 | Rv1736c | 462 | [symbol] | 0.46 | 0.78 |
| A24 | Rv1736c | 513 | [symbol] | 0.46 | 0.78 |
| A24 | Rv1736c | 468 | [symbol] | 0.47 | 0.77 |
| A24 | Rv1736c | 424 | [symbol] | 0.46 | 0.76 |
| A24 | Rv1736c | 422 | [symbol] | 0.52 | 0.76 |
| A24 | Rv1736c | 73 | [symbol] | 0.44 | 0.76 |
| B7 | Rv1736c | 401 | [symbol] | 0.53 | 1.55 |
| B7 | Rv1736c | 103 | [symbol] | 0.46 | 1.40 |
| B7 | Rv1736c | 485 | [symbol] | 0.46 | 1.33 |
| B7 | Rv1736c | 425 | [symbol] | 0.42 | 1.29 |
| B7 | Rv1736c | 161 | [symbol] | 0.41 | 1.21 |
| B7 | Rv1736c | 200 | [symbol] | 0.39 | 1.19 |
| B7 | Rv1736c | 583 | [symbol] | 0.35 | 1.12 |
| B7 | Rv1736c | 121 | [symbol] | 0.36 | 1.10 |
| B7 | Rv1736c | 530 | [symbol] | 0.36 | 1.09 |
| B7 | Rv1736c | 465 | [symbol] | 0.34 | 1.08 |
| B7 | Rv1736c | 15 | [symbol] | 0.37 | 1.07 |
| B7 | Rv1736c | 180 | [symbol] | 0.33 | 1.05 |
| B7 | Rv1736c | 424 | [symbol] | 0.32 | 1.02 |
| B7 | Rv1736c | 610 | [symbol] | 0.31 | 1.01 |
| B7 | Rv1736c | 552 | [symbol] | 0.29 | 0.96 |
| B7 | Rv1736c | 120 | [symbol] | 0.31 | 0.93 |
| B7 | Rv1736c | 257 | [symbol] | 0.28 | 0.92 |
| B7 | Rv1736c | 152 | [symbol] | 0.33 | 0.92 |
| B7 | Rv1736c | 374 | [symbol] | 0.28 | 0.89 |
| B7 | Rv1736c | 525 | [symbol] | 0.25 | 0.88 |
| B7 | Rv1736c | 202 | [symbol] | 0.32 | 0.87 |
| B7 | Rv1736c | 199 | [symbol] | 0.29 | 0.85 |
| B7 | Rv1736c | 363 | [symbol] | 0.28 | 0.84 |
| B7 | Rv1736c | 47 | [symbol] | 0.27 | 0.83 |
| B7 | Rv1736c | 644 | [symbol] | 0.26 | 0.79 |
| B7 | Rv1736c | 631 | [symbol] | 0.23 | 0.78 |
| B8 | Rv1736c | 123 | [symbol] | 0.27 | 1.43 |
| B8 | Rv1736c | 438 | [symbol] | 0.23 | 1.27 |
| B8 | Rv1736c | 539 | [symbol] | 0.21 | 1.10 |
| B8 | Rv1736c | 163 | [symbol] | 0.19 | 1.05 |
| B8 | Rv1736c | 377 | [symbol] | 0.17 | 0.99 |
| B8 | Rv1736c | 131 | [symbol] | 0.16 | 0.94 |
| B8 | Rv1736c | 614 | [symbol] | 0.16 | 0.91 |
| B8 | Rv1736c | 524 | [symbol] | 0.17 | 0.86 |
| B8 | Rv1736c | 384 | [symbol] | 0.14 | 0.86 |
| B8 | Rv1736c | 596 | [symbol] | 0.15 | 0.83 |
| B8 | Rv1736c | 191 | [symbol] | 0.14 | 0.81 |
| B8 | Rv1736c | 73 | [symbol] | 0.13 | 0.80 |
| B8 | Rv1736c | 599 | [symbol] | 0.13 | 0.78 |
| B8 | Rv1736c | 354 | [symbol] | 0.13 | 0.77 |
| B8 | Rv1736c | 636 | [symbol] | 0.12 | 0.75 |
| B27 | Rv1736c | 157 | [symbol] | 0.63 | 3.16 |
| B27 | Rv1736c | 319 | [symbol] | 0.52 | 2.82 |
| B27 | Rv1736c | 525 | [symbol] | 0.42 | 2.23 |
| B27 | Rv1736c | 155 | [symbol] | 0.39 | 2.02 |
| B27 | Rv1736c | 379 | [symbol] | 0.34 | 1.79 |
| B27 | Rv1736c | 526 | [symbol] | 0.33 | 1.77 |
| B27 | Rv1736c | 293 | [symbol] | 0.29 | 1.59 |
| B27 | Rv1736c | 104 | [symbol] | 0.26 | 1.51 |
| B27 | Rv1736c | 160 | [symbol] | 0.26 | 1.41 |
| B27 | Rv1736c | 19 | [symbol] | 0.25 | 1.34 |
| B27 | Rv1736c | 9 | [symbol] | 0.22 | 1.21 |
| B27 | Rv1736c | 571 | [symbol] | 0.20 | 1.21 |
| B27 | Rv1736c | 378 | [symbol] | 0.22 | 1.19 |
| B27 | Rv1736c | 333 | [symbol] | 0.19 | 1.07 |
| B27 | Rv1736c | 124 | [symbol] | 0.16 | 1.03 |
| B27 | Rv1736c | 309 | [symbol] | 0.17 | 1.03 |
| B27 | Rv1736c | 335 | [symbol] | 0.16 | 0.97 |
| B27 | Rv1736c | 154 | [symbol] | 0.17 | 0.93 |
| B27 | Rv1736c | 153 | [symbol] | 0.14 | 0.83 |
| B27 | Rv1736c | 156 | [symbol] | 0.17 | 0.82 |
| B27 | Rv1736c | 461 | [symbol] | 0.16 | 0.80 |
| B27 | Rv1736c | 300 | [symbol] | 0.14 | 0.80 |
| B27 | Rv1736c | 162 | [symbol] | 0.13 | 0.80 |
| B27 | Rv1736c | 36 | [symbol] | 0.13 | 0.76 |
| B44 | Rv1736c | 566 | [symbol] | 0.19 | 1.27 |
| B44 | Rv1736c | 177 | [symbol] | 0.15 | 1.18 |
| B44 | Rv1736c | 338 | [symbol] | 0.16 | 1.11 |
| B44 | Rv1736c | 572 | [symbol] | 0.14 | 1.06 |
| B44 | Rv1736c | 129 | [symbol] | 0.14 | 1.01 |
| B44 | Rv1736c | 297 | [symbol] | 0.13 | 0.95 |
| B44 | Rv1736c | 12 | [symbol] | 0.13 | 0.94 |
| B44 | Rv1736c | 277 | [symbol] | 0.14 | 0.86 |
| B44 | Rv1736c | 418 | [symbol] | 0.12 | 0.81 |
| B44 | Rv1736c | 81 | [symbol] | 0.12 | 0.81 |
| B44 | Rv1736c | 174 | [symbol] | 0.12 | 0.81 |
| B44 | Rv1736c | 640 | [symbol] | 0.10 | 0.79 |
| B44 | Rv1736c | 367 | [symbol] | 0.11 | 0.78 |
| B58 | Rv1736c | 644 | [symbol] | 0.69 | 2.46 |
| B58 | Rv1736c | 430 | [symbol] | 0.66 | 2.33 |
| B58 | Rv1736c | 481 | [symbol] | 0.61 | 2.20 |
| B58 | Rv1736c | 575 | [symbol] | 0.56 | 2.10 |
| B58 | Rv1736c | 320 | [symbol] | 0.56 | 2.10 |
| B58 | Rv1736c | 622 | [symbol] | 0.54 | 2.00 |
| B58 | Rv1736c | 247 | [symbol] | 0.50 | 1.77 |
| B58 | Rv1736c | 431 | [symbol] | 0.46 | 1.66 |
| B58 | Rv1736c | 165 | [symbol] | 0.43 | 1.61 |
| B58 | Rv1736c | 138 | [symbol] | 0.39 | 1.51 |
| B58 | Rv1736c | 246 | [symbol] | 0.42 | 1.50 |
| B58 | Rv1736c | 620 | [symbol] | 0.32 | 1.37 |
| B58 | Rv1736c | 62 | [symbol] | 0.33 | 1.26 |
| B58 | Rv1736c | 448 | [symbol] | 0.28 | 1.21 |
| B58 | Rv1736c | 222 | [symbol] | 0.27 | 1.06 |
| B58 | Rv1736c | 412 | [symbol] | 0.27 | 0.99 |
| B58 | Rv1736c | 561 | [symbol] | 0.23 | 0.98 |
| B58 | Rv1736c | 591 | [symbol] | 0.21 | 0.97 |
| B58 | Rv1736c | 251 | [symbol] | 0.24 | 0.96 |
| B58 | Rv1736c | 208 | [symbol] | 0.23 | 0.94 |
| B58 | Rv1736c | 513 | [symbol] | 0.23 | 0.94 |
| B58 | Rv1736c | 266 | [symbol] | 0.22 | 0.92 |
| B58 | Rv1736c | 605 | [symbol] | 0.24 | 0.89 |
| B58 | Rv1736c | 414 | [symbol] | 0.25 | 0.88 |
| B58 | Rv1736c | 433 | [symbol] | 0.16 | 0.81 |
| B62 | Rv1736c | 628 | [symbol] | 0.49 | 1.50 |
| B62 | Rv1736c | 339 | [symbol] | 0.45 | 1.41 |
| B62 | Rv1736c | 460 | [symbol] | 0.43 | 1.31 |
| B62 | Rv1736c | 419 | [symbol] | 0.41 | 1.28 |
| B62 | Rv1736c | 591 | [symbol] | 0.40 | 1.27 |
| B62 | Rv1736c | 219 | [symbol] | 0.40 | 1.24 |
| B62 | Rv1736c | 316 | [symbol] | 0.39 | 1.22 |
| B62 | Rv1736c | 448 | [symbol] | 0.38 | 1.20 |
| B62 | Rv1736c | 395 | [symbol] | 0.35 | 1.16 |
| B62 | Rv1736c | 191 | [symbol] | 0.38 | 1.12 |
| B62 | Rv1736c | 138 | [symbol] | 0.37 | 1.11 |
| B62 | Rv1736c | 14 | [symbol] | 0.37 | 1.08 |
| B62 | Rv1736c | 622 | [symbol] | 0.36 | 1.07 |
| B62 | Rv1736c | 599 | [symbol] | 0.35 | 1.03 |
| B62 | Rv1736c | 563 | [symbol] | 0.31 | 1.03 |
| B62 | Rv1736c | 561 | [symbol] | 0.32 | 1.02 |
| B62 | Rv1736c | 90 | [symbol] | 0.28 | 0.95 |
| B62 | Rv1736c | 101 | [symbol] | 0.32 | 0.94 |
| B62 | Rv1736c | 590 | [symbol] | 0.29 | 0.90 |
| B62 | Rv1736c | 106 | [symbol] | 0.24 | 0.87 |
| B62 | Rv1736c | 344 | [symbol] | 0.28 | 0.85 |
| B62 | Rv1736c | 223 | [symbol] | 0.23 | 0.83 |
| B62 | Rv1736c | 234 | [symbol] | 0.24 | 0.83 |
| B62 | Rv1736c | 13 | [symbol] | 0.23 | 0.80 |
| B62 | Rv1736c | 453 | [symbol] | 0.27 | 0.78 |
| B62 | Rv1736c | 220 | [symbol] | 0.25 | 0.78 |
| B62 | Rv1736c | 38 | [symbol] | 0.21 | 0.77 |
| B62 | Rv1736c | 584 | [symbol] | 0.26 | 0.77 |
| B62 | Rv1736c | 73 | [symbol] | 0.24 | 0.76 |
| B62 | Rv1736c | 620 | [symbol] | 0.20 | 0.76 |
| B62 | Rv1736c | 516 | [symbol] | 0.20 | 0.75 |

Predicted supertype HLA class-I
epitopes in RV1737c (NarK2)

| | | | | | |
|---|---|---|---|---|---|
| A1 | Rv1737c | 355 [symbol] | 0.50 | 3.66 | |
| A1 | Rv1737c | 372 | [symbol] | 0.31 | 2.37 |
| A1 | Rv1737c | 207 | [symbol] | 0.20 | 1.63 |
| A1 | Rv1737c | 89 | [symbol] | 0.18 | 1.44 |
| A1 | Rv1737c | 64 | [symbol] | 0.20 | 1.35 |
| A1 | Rv1737c | 265 | [symbol] | 0.16 | 1.06 |
| A1 | Rv1737c | 211 | [symbol] | 0.12 | 0.99 |
| A1 | Rv1737c | 146 | [symbol] | 0.11 | 0.98 |
| A1 | Rv1737c | 298 | [symbol] | 0.09 | 0.77 |
| A1 | Rv1737c | 224 | [symbol] | 0.07 | 0.77 |
| A2 | Rv1737c | 108 | [symbol] | 0.53 | 1.33 |
| A2 | Rv1737c | 228 | [symbol] | 0.50 | 1.33 |
| A2 | Rv1737c | 9 | [symbol] | 0.49 | 1.29 |
| A2 | Rv1737c | 368 | [symbol] | 0.40 | 1.07 |
| A2 | Rv1737c | 283 | [symbol] | 0.36 | 1.01 |
| A2 | Rv1737c | 159 | [symbol] | 0.37 | 1.00 |
| A2 | Rv1737c | 342 | [symbol] | 0.32 | 0.91 |
| A2 | Rv1737c | 351 | [symbol] | 0.37 | 0.90 |
| A2 | Rv1737c | 51 | [symbol] | 0.32 | 0.90 |
| A2 | Rv1737c | 82 | [symbol] | 0.32 | 0.89 |
| A2 | Rv1737c | 93 | [symbol] | 0.31 | 0.86 |
| A2 | Rv1737c | 363 | [symbol] | 0.31 | 0.84 |
| A2 | Rv1737c | 264 | [symbol] | 0.28 | 0.83 |
| A2 | Rv1737c | 101 | [symbol] | 0.28 | 0.81 |
| A2 | Rv1737c | 213 | [symbol] | 0.35 | 0.78 |
| A2 | Rv1737c | 313 | [symbol] | 0.28 | 0.78 |
| A2 | Rv1737c | 140 | [symbol] | 0.29 | 0.77 |
| A2 | Rv1737c | 16 | [symbol] | 0.27 | 0.75 |
| A2 | Rv1737c | 78 | [symbol] | 0.25 | 0.75 |
| A3 | Rv1737c | 316 | [symbol] | 0.43 | 1.45 |
| A3 | Rv1737c | 145 | [symbol] | 0.39 | 1.33 |
| A3 | Rv1737c | 378 | [symbol] | 0.35 | 1.20 |
| A3 | Rv1737c | 372 | [symbol] | 0.31 | 1.17 |
| A3 | Rv1737c | 175 | [symbol] | 0.31 | 1.02 |
| A3 | Rv1737c | 179 | [symbol] | 0.22 | 0.93 |
| A3 | Rv1737c | 217 | [symbol] | 0.21 | 0.88 |
| A3 | Rv1737c | 112 | [symbol] | 0.20 | 0.82 |
| A3 | Rv1737c | 117 | [symbol] | 0.18 | 0.79 |
| A3 | Rv1737c | 24 | [symbol] | 0.17 | 0.76 |
| A24 | Rv1737c | 214 | [symbol] | 0.86 | 1.39 |
| A24 | Rv1737c | 124 | [symbol] | 0.87 | 1.38 |
| A24 | Rv1737c | 231 | [symbol] | 0.83 | 1.37 |
| A24 | Rv1737c | 149 | [symbol] | 0.81 | 1.25 |
| A24 | Rv1737c | 153 | [symbol] | 0.75 | 1.19 |
| A24 | Rv1737c | 355 | [symbol] | 0.62 | 1.11 |
| A24 | Rv1737c | 221 | [symbol] | 0.72 | 1.11 |
| A24 | Rv1737c | 362 | [symbol] | 0.71 | 1.10 |
| A24 | Rv1737c | 97 | [symbol] | 0.69 | 1.10 |
| A24 | Rv1737c | 141 | [symbol] | 0.70 | 1.09 |
| A24 | Rv1737c | 150 | [symbol] | 0.69 | 1.07 |
| A24 | Rv1737c | 98 | [symbol] | 0.62 | 1.05 |
| A24 | Rv1737c | 279 | [symbol] | 0.59 | 1.03 |
| A24 | Rv1737c | 298 | [symbol] | 0.64 | 1.02 |
| A24 | Rv1737c | 302 | [symbol] | 0.65 | 1.02 |
| A24 | Rv1737c | 106 | [symbol] | 0.61 | 1.01 |
| A24 | Rv1737c | 217 | [symbol] | 0.55 | 1.00 |
| A24 | Rv1737c | 131 | [symbol] | 0.62 | 0.97 |
| A24 | Rv1737c | 375 | [symbol] | 0.59 | 0.97 |
| A24 | Rv1737c | 11 | [symbol] | 0.54 | 0.95 |
| A24 | Rv1737c | 14 | [symbol] | 0.59 | 0.94 |
| A24 | Rv1737c | 367 | [symbol] | 0.58 | 0.94 |
| A24 | Rv1737c | 105 | [symbol] | 0.59 | 0.94 |
| A24 | Rv1737c | 16 | [symbol] | 0.58 | 0.90 |
| A24 | Rv1737c | 12 | [symbol] | 0.57 | 0.90 |
| A24 | Rv1737c | 271 | [symbol] | 0.54 | 0.89 |
| A24 | Rv1737c | 95 | [symbol] | 0.54 | 0.88 |
| A24 | Rv1737c | 146 | [symbol] | 0.48 | 0.88 |
| A24 | Rv1737c | 268 | [symbol] | 0.50 | 0.85 |
| A24 | Rv1737c | 224 | [symbol] | 0.42 | 0.84 |
| A24 | Rv1737c | 228 | [symbol] | 0.51 | 0.83 |
| A24 | Rv1737c | 180 | [symbol] | 0.45 | 0.81 |
| A24 | Rv1737c | 71 | [symbol] | 0.46 | 0.79 |
| A24 | Rv1737c | 205 | [symbol] | 0.41 | 0.78 |
| A24 | Rv1737c | 20 | [symbol] | 0.46 | 0.78 |
| A24 | Rv1737c | 67 | [symbol] | 0.51 | 0.77 |
| A24 | Rv1737c | 283 | [symbol] | 0.45 | 0.77 |
| A24 | Rv1737c | 94 | [symbol] | 0.47 | 0.77 |
| A24 | Rv1737c | 211 | [symbol] | 0.44 | 0.76 |
| A24 | Rv1737c | 99 | [symbol] | 0.44 | 0.76 |
| A24 | Rv1737c | 187 | [symbol] | 0.51 | 0.76 |
| A24 | Rv1737c | 48 | [symbol] | 0.48 | 0.76 |
| B7 | Rv1737c | 325 | [symbol] | 0.57 | 1.64 |
| B7 | Rv1737c | 245 | [symbol] | 0.51 | 1.58 |
| B7 | Rv1737c | 276 | [symbol] | 0.49 | 1.48 |
| B7 | Rv1737c | 168 | [symbol] | 0.52 | 1.46 |
| B7 | Rv1737c | 126 | [symbol] | 0.51 | 1.38 |
| B7 | Rv1737c | 257 | [symbol] | 0.45 | 1.35 |
| B7 | Rv1737c | 347 | [symbol] | 0.51 | 1.35 |
| B7 | Rv1737c | 196 | [symbol] | 0.43 | 1.28 |
| B7 | Rv1737c | 375 | [symbol] | 0.39 | 1.26 |
| B7 | Rv1737c | 71 | [symbol] | 0.37 | 1.20 |
| B7 | Rv1737c | 171 | [symbol] | 0.39 | 1.20 |
| B7 | Rv1737c | 205 | [symbol] | 0.35 | 1.19 |
| B7 | Rv1737c | 270 | [symbol] | 0.44 | 1.10 |
| B7 | Rv1737c | 268 | [symbol] | 0.34 | 1.09 |
| B7 | Rv1737c | 83 | [symbol] | 0.36 | 1.04 |
| B7 | Rv1737c | 193 | [symbol] | 0.33 | 0.98 |
| B7 | Rv1737c | 65 | [symbol] | 0.33 | 0.94 |
| B7 | Rv1737c | 56 | [symbol] | 0.27 | 0.90 |
| B7 | Rv1737c | 320 | [symbol] | 0.32 | 0.88 |
| B7 | Rv1737c | 292 | [symbol] | 0.29 | 0.86 |
| B7 | Rv1737c | 2 | [symbol] | 0.26 | 0.85 |
| B7 | Rv1737c | 86 | [symbol] | 0.25 | 0.81 |
| B7 | Rv1737c | 318 | [symbol] | 0.27 | 0.76 |
| B8 | Rv1737c | 66 | [symbol] | 0.16 | 0.97 |
| B8 | Rv1737c | 187 | [symbol] | 0.18 | 0.93 |
| B8 | Rv1737c | 56 | [symbol] | 0.14 | 0.83 |
| B8 | Rv1737c | 318 | [symbol] | 0.14 | 0.80 |
| B27 | Rv1737c | 66 | [symbol] | 0.43 | 2.27 |
| B27 | Rv1737c | 129 | [symbol] | 0.37 | 2.05 |
| B27 | Rv1737c | 203 | [symbol] | 0.23 | 1.27 |
| B27 | Rv1737c | 152 | [symbol] | 0.22 | 1.21 |
| B27 | Rv1737c | 322 | [symbol] | 0.17 | 0.96 |
| B27 | Rv1737c | 197 | [symbol] | 0.15 | 0.90 |
| B27 | Rv1737c | 217 | [symbol] | 0.13 | 0.80 |
| B44 | Rv1737c | 295 | [symbol] | 0.34 | 2.42 |
| B44 | Rv1737c | 209 | [symbol] | 0.16 | 1.13 |
| B58 | Rv1737c | 14 | [symbol] | 0.75 | 2.72 |
| B58 | Rv1737c | 207 | [symbol] | 0.62 | 2.40 |
| B58 | Rv1737c | 116 | [symbol] | 0.60 | 2.18 |
| B58 | Rv1737c | 290 | [symbol] | 0.48 | 1.83 |
| B58 | Rv1737c | 256 | [symbol] | 0.46 | 1.73 |
| B58 | Rv1737c | 60 | [symbol] | 0.37 | 1.41 |
| B58 | Rv1737c | 312 | [symbol] | 0.37 | 1.39 |
| B58 | Rv1737c | 39 | [symbol] | 0.35 | 1.32 |
| B58 | Rv1737c | 201 | [symbol] | 0.35 | 1.31 |
| B58 | Rv1737c | 133 | [symbol] | 0.27 | 0.95 |
| B58 | Rv1737c | 30 | [symbol] | 0.22 | 0.90 |
| B58 | Rv1737c | 211 | [symbol] | 0.18 | 0.80 |
| B58 | Rv1737c | 11 | [symbol] | 0.16 | 0.75 |
| B62 | Rv1737c | 24 | [symbol] | 0.55 | 1.66 |
| B62 | Rv1737c | 106 | [symbol] | 0.50 | 1.45 |
| B62 | Rv1737c | 89 | [symbol] | 0.45 | 1.39 |
| B62 | Rv1737c | 179 | [symbol] | 0.41 | 1.31 |
| B62 | Rv1737c | 217 | [symbol] | 0.42 | 1.31 |
| B62 | Rv1737c | 207 | [symbol] | 0.35 | 1.15 |
| B62 | Rv1737c | 124 | [symbol] | 0.37 | 1.13 |
| B62 | Rv1737c | 290 | [symbol] | 0.35 | 1.06 |
| B62 | Rv1737c | 112 | [symbol] | 0.32 | 1.04 |
| B62 | Rv1737c | 98 | [symbol] | 0.29 | 0.94 |
| B62 | Rv1737c | 229 | [symbol] | 0.27 | 0.91 |
| B62 | Rv1737c | 372 | [symbol] | 0.22 | 0.81 |
| B62 | Rv1737c | 367 | [symbol] | 0.26 | 0.81 |
| B62 | Rv1737c | 310 | [symbol] | 0.24 | 0.79 |
| B62 | Rv1737c | 141 | [symbol] | 0.26 | 0.78 |
| B62 | Rv1737c | 220 | [symbol] | 0.22 | 0.78 |
| B62 | Rv1737c | 95 | [symbol] | 0.24 | 0.76 |
| B62 | Rv1737c | 283 | [symbol] | 0.24 | 0.76 |

[symbol]

indicates data missing or illegible when filed

TABLE 5
Recognition of 20-mer peptides from TD latency antigens

| | | | | CD4 | | | CD8 | | |
|---|---|---|---|---|---|---|---|---|---|
| peptide | [symbol] | start | end | >20-50% | >50-75% | >75% | >20-50% | >50-75% | >75% |

| | | | | | | | | | |
|---|---|---|---|---|---|---|---|---|---|
| 1 | [symbol] | 1 | 20 | | | [symbol] | [symbol] | | [symbol] |
| 2 | [symbol] | 11 | 30 | [symbol] | | [symbol] | | [symbol] | [symbol] |
| 3 | [symbol] | 21 | 40 | [symbol] | | | [symbol] | | |
| 4 | [symbol] | 31 | 50 | [symbol] | | | [symbol] | | |
| 5 | [symbol] | 41 | 60 | | | | | | |
| 6 | [symbol] | 51 | 70 | | | | [symbol] | | |
| 7 | [symbol] | 61 | 80 | | [symbol] | | | [symbol] | |
| 8 | [symbol] | 71 | 90 | | | | | [symbol] | |
| 9 | [symbol] | 81 | 100 | | | | | | |
| 10 | [symbol] | 91 | 110 | | | | | | |
| 11 | [symbol] | 101 | 120 | | | | | [symbol] | |
| 12 | [symbol] | 111 | 130 | [symbol] | | | | | |
| 13 | [symbol] | 121 | 140 | | | | | | |
| 14 | [symbol] | 131 | 150 | | | | | | |
| 15 | [symbol] | 141 | 160 | [symbol] | [symbol] | | [symbol] | | |
| 16 | [symbol] | 151 | 170 | | | | | | |
| 17 | [symbol] | 161 | 180 | | [symbol] | | [symbol] | [symbol] | |
| 18 | [symbol] | 171 | 190 | | | | | | |
| 19 | [symbol] | 181 | 200 | [symbol] | | | [symbol] | [symbol] | |
| 20 | [symbol] | 191 | 210 | | | | | | |

| | | | | CD4 | | | CD8 | | |
|---|---|---|---|---|---|---|---|---|---|
| peptide | [symbol] | start | end | >20-50% | >50-75% | >75% | >20-50% | >50-75% | >75% |

| | | | | | | | | | |
|---|---|---|---|---|---|---|---|---|---|
| 1 | [symbol] | 1 | 20 | | | | | | |
| 2 | [symbol] | 11 | 30 | [symbol] | | | | | |
| 3 | [symbol] | 21 | 40 | | | | | | |
| 4 | [symbol] | 31 | 50 | [symbol] | | | [symbol] | | |
| 5 | [symbol] | 41 | 60 | | | | | | |
| 6 | [symbol] | 51 | 70 | [symbol] | | | | | [symbol] |
| 7 | [symbol] | 61 | 80 | [symbol] | | | | | [symbol] |
| 8 | [symbol] | 71 | 90 | | | | | | |
| 9 | [symbol] | 81 | 100 | [symbol] | | | [symbol] | | |
| 10 | [symbol] | 91 | 110 | | | | | | |
| 11 | [symbol] | 101 | 120 | | [symbol] | | [symbol] | | |
| 12 | [symbol] | 111 | 130 | [symbol] | | | | | |
| 13 | [symbol] | 121 | 140 | | | | | | |
| 14 | [symbol] | 131 | 150 | [symbol] | | | | [symbol] | |
| 15 | [symbol] | 141 | 160 | [symbol] | | | | | |
| 16 | [symbol] | 151 | 170 | | | | [symbol] | | |
| 17 | [symbol] | 161 | 180 | | | | | | |
| 18 | [symbol] | 171 | 190 | | | | | | |
| 19 | [symbol] | 181 | 200 | | | | [symbol] | | |
| 20 | [symbol] | 191 | 210 | | | | [symbol] | | |
| 21 | [symbol] | 201 | 220 | | | | | | |
| 22 | [symbol] | 211 | 230 | [symbol] | | | | | |
| 23 | [symbol] | 221 | 240 | | [symbol] | | | | |
| 24 | [symbol] | 231 | 250 | [symbol] | | | | | |
| 25 | [symbol] | 241 | 260 | | | | | | |
| 26 | [symbol] | 251 | 270 | [symbol] | | | | | |
| 27 | [symbol] | 261 | 280 | | | | | | |
| 28 | [symbol] | 271 | 290 | | [symbol] | [symbol] | | | |
| 29 | [symbol] | 281 | 300 | [symbol] | | | | | |
| 30 | [symbol] | 291 | 310 | | | | | | |
| 31 | [symbol] | 301 | 320 | [symbol] | | | | | |
| 32 | [symbol] | 311 | 330 | [symbol] | | | | | |
| 33 | [symbol] | 318 | 339 | | | | | | |

| | | | | CD4 | | | CD8 | | |
|---|---|---|---|---|---|---|---|---|---|
| peptide | [symbol] | start | end | >20-50% | >50-75% | >75% | >20-50% | >50-75% | >75% |

| | | | | | | |
|---|---|---|---|---|---|---|
| 1 | [symbol] | 1 | 20 | | | |
| 2 | [symbol] | 11 | 30 | | | |
| 3 | [symbol] | 21 | 40 | | | |
| 4 | [symbol] | 31 | 50 | | | |
| 5 | [symbol] | 41 | 60 | | | |
| 6 | [symbol] | 51 | 70 | | | |
| 7 | [symbol] | 61 | 80 | | | [symbol] |
| 8 | [symbol] | 71 | 90 | | | |
| 9 | [symbol] | 81 | 100 | | | [symbol] |
| 10 | [symbol] | 91 | 110 | | | |
| 11 | [symbol] | 101 | 120 | | | |
| 12 | [symbol] | 111 | 130 | [symbol] | | |
| 13 | [symbol] | 121 | 140 | | | |
| 14 | [symbol] | 131 | 150 | | | |
| 15 | [symbol] | 141 | 160 | | | |
| 16 | [symbol] | 151 | 170 | | | [symbol] |
| 17 | [symbol] | 161 | 180 | | | [symbol] |
| 18 | [symbol] | 171 | 190 | | | |
| 19 | [symbol] | 181 | 200 | | [symbol] | |
| 20 | [symbol] | 191 | 210 | | [symbol] | |
| 21 | [symbol] | 201 | 220 | | [symbol] | |
| 22 | [symbol] | 211 | 230 | [symbol] | | |
| 23 | [symbol] | 221 | 240 | [symbol] | [symbol] | [symbol] |
| 24 | [symbol] | 231 | 250 | | | |
| 25 | [symbol] | 241 | 260 | [symbol] | | |
| 26 | [symbol] | 251 | 270 | | | |
| 27 | [symbol] | 261 | 280 | | | |
| 28 | [symbol] | 271 | 290 | | [symbol] | |
| 29 | [symbol] | 281 | 300 | | | |
| 30 | [symbol] | 291 | 310 | | | |
| 31 | [symbol] | 301 | 320 | | | |
| 32 | [symbol] | 311 | 330 | [symbol] | | |
| 33 | [symbol] | 321 | 340 | | | |
| 34 | [symbol] | 331 | 350 | | | |
| 35 | [symbol] | 341 | 360 | [symbol] | [symbol] | [symbol] |
| 36 | [symbol] | 351 | 370 | | [symbol] | |
| 37 | [symbol] | 361 | 380 | | [symbol] | |
| 38 | [symbol] | 371 | 390 | [symbol] | | |
| 39 | [symbol] | 381 | 400 | | [symbol] | |
| 40 | [symbol] | 391 | 410 | | | |
| 41 | [symbol] | 394 | 413 | | | |

| | | | | CD4 | | | CD8 | | |
|---|---|---|---|---|---|---|---|---|---|
| peptide | [symbol] | start | end | >20-50% | >50-75% | >75% | >20-50% | >50-75% | >75% |

| | | | | | |
|---|---|---|---|---|---|
| 1 | [symbol] | 1 | 20 | | |
| 2 | [symbol] | 11 | 30 | | |
| 3 | [symbol] | 21 | 40 | | [symbol] |
| 4 | [symbol] | 31 | 50 | [symbol] | |
| 5 | [symbol] | 41 | 60 | [symbol] | [symbol] |
| 6 | [symbol] | 51 | 70 | | |
| 7 | [symbol] | 61 | 80 | | |
| 8 | [symbol] | 71 | 90 | | |
| 9 | [symbol] | 81 | 100 | [symbol] | |
| 10 | [symbol] | 91 | 110 | | |
| 11 | [symbol] | 101 | 120 | | [symbol] |

[symbol]

indicates data missing or illegible when filed

TABLE 6

Predicted linear and conformational
B-cell epitopes in TB latency antigens

[symbol]

| | |
|---|---|
| 1 | [symbol] |
| 61 | [symbol] |
| 121 | [symbol] |
| 181 | [symbol] |

[symbol]

| | |
|---|---|
| 1 | [symbol] |
| 61 | [symbol] |
| 121 | [symbol] |
| 181 | [symbol] |
| 241 | [symbol] |
| 301 | [symbol] |

[symbol]

| | |
|---|---|
| 1 | [symbol] |
| 61 | [symbol] |
| 121 | [symbol] |
| 181 | [symbol] |
| 241 | [symbol] |
| 301 | [symbol] |
| 361 | [symbol] |

[symbol]

| | |
|---|---|
| 1 | [symbol] |
| 61 | [symbol] |

[symbol]

| | |
|---|---|
| 1 | [symbol] |
| 61 | [symbol] |
| 121 | [symbol] |

[symbol]

| | |
|---|---|
| 1 | [symbol] |
| 61 | [symbol] |
| 121 | [symbol] |

[symbol]

| | |
|---|---|
| 1 | [symbol] |
| 61 | [symbol] |
| 121 | [symbol] |
| 181 | [symbol] |
| 241 | [symbol] |
| 301 | [symbol] |
| 361 | [symbol] |
| 421 | [symbol] |
| 481 | [symbol] |
| 541 | [symbol] |
| 601 | [symbol] |

[symbol]

| | |
|---|---|
| 1 | [symbol] |
| 61 | [symbol] |
| 121 | [symbol] |
| 181 | [symbol] |
| 241 | [symbol] |
| 301 | [symbol] |
| 361 | [symbol] |

[symbol]

indicates data missing or illegible when filed

### REFERENCES

1. Wayne, L. G. and H. A. Sramek. 1994. Metronidazole is bactericidal to dormant cells of Mycobacterium tuberculosis. Antimicrob. Agents Chemother. 38:2054-2058.
2. Wayne, L. G. and K. Y. Lin. 1982. Glyoxylate metabolism and adaptation of Mycobacterium tuberculosis to survival under anaerobic conditions. Infect. Immun. 37:1042-1049.
3. Lillebaek, T., A. Dirksen, E. Vynnycky, I. Baess, V. O. Thomsen, and A. B. Andersen. 2003. Stability of DNA patterns and evidence of Mycobacterium tuberculosis reactivation occurring decades after the initial infection. J. Infect. Dis. 188:1032-1039.
4. Warren, R. M., G. D. van der Spuy, M. Richardson, N. Beyers, M. W. Borgdorff, M. A. Behr, and P. D. van Helden. 2002. Calculation of the stability of the IS6110 banding pattern in patients with persistent Mycobacterium tuberculosis disease. J. Clin. Microbiol. 40:1705-1708.
5. Fine, P. E. 1995. Variation in protection by BCG: implications of and for heterologous immunity. Lancet 346:1339-1345.
6. Turner, J., E. R. Rhoades, M. Keen, J. T. Belisle, A. A. Frank, and I. M. Orme. 2000. Effective preexposure tuberculosis vaccines fail to protect when they are given in an immunotherapeutic mode. Infect. Immun. 68:1706-1709.
7. Moreira, A. L., L. Tsenova, M. H. Aman, L. G. Bekker, S. Freeman, B. Mangaliso, U. Schroder, J. Jagirdar, W. N. Rom, M. G. Tovey, V. H. Freedman, and G. Kaplan. 2002. Mycobacterial antigens exacerbate disease manifestations in Mycobacterium tuberculosis-infected mice. Infect. Immun. 70:2100-2107.
8. Repique, C. J., A. Li, F. M. Collins, and S. L. Morris. 2002. DNA immunization in a mouse model of latent tuberculosis: effect of DNA vaccination on reactivation of disease and on reinfection with a secondary challenge. Infect. Immun. 70:3318-3323.
9. Chan, J. and J. Flynn. 2004. The immunological aspects of latency in tuberculosis. Clin. Immunol. 110:2-12.
10. Cunningham, A. F. and C. L. Spreadbury. 1998. Mycobacterial stationary phase induced by low oxygen tension: cell wall thickening and localization of the 16-kilodalton alpha-crystallin homolog. J. Bacteriol. 180:801-808.
11. Yuan, Y., D. D. Crane, and C. E. Barry, III. 1996. Stationary phase-associated protein expression in Mycobacterium tuberculosis: function of the mycobacterial alpha-crystallin homolog. J. Bacteriol. 178:4484-4492.
12. Wilkinson, R. J., K. A. Wilkinson, K. A. De Smet, K. Haslov, G. Pasvol, M. Singh, I. Svarcova, and J. Ivanyi. 1998. Human T- and B-cell reactivity to the 16 kDa alpha-crystallin protein of Mycobacterium tuberculosis. Scand. J. Immunol. 48:403-409.
13. Oftung, F., E. Borka, and A. S. Mustafa. 1998. Mycobacterium tuberculosis reactive T cell clones from naturally converted PPD-positive healthy subjects: recognition of the M. tuberculosis 16-kDa antigen. FEMS Immunol. Med. Microbiol. 20:319-325.
14. Wayne, L. G. and L. G. Hayes. 1996. An in vitro model for sequential study of shiftdown of Mycobacterium tuberculosis through two stages of nonreplicating persistence. Infect. Immun. 64:2062-2069.
15. Sherman, D. R., M. Voskuil, D. Schnappinger, R. Liao, M. I. Harrell, and G. K. Schoolnik. 2001. Regulation of the Mycobacterium tuberculosis hypoxic response gene encoding alpha-crystallin. Proc. Natl. Acad. Sci. U.S.A 98:7534-7539.
16. Rosenkrands, I., R. A. Slayden, J. Crawford, C. Aagaard, C. E. Barry, 111, and P. Andersen. 2002. Hypoxic response of Mycobacterium tuberculosis studied by metabolic labeling and proteome analysis of cellular and extracellular proteins. J. Bacteriol. 184:3485-3491.
17. Voskuil, M. I., D. Schnappinger, K. C. Visconti, M. I. Harrell, G. M. Dolganov, D. R. Sherman, and G. K. Schoolnik. 2003. Inhibition of respiration by nitric oxide induces a Mycobacterium tuberculosis dormancy program. J. Exp. Med. 198:705-713.
18. Schnappinger, D., S. Ehrt, M. I. Voskuil, Y. Liu, J. A. Mangan, I. M. Monahan, G. Dolganov, B. Efron, P. D. Butcher, C. Nathan, and G. K. Schoolnik. 2003. Transcriptional Adaptation of Mycobacterium tuberculosis within Macrophages: Insights into the Phagosomal Environment. J. Exp. Med. 198:693-704.
19. Shi, L., Y. J. Jung, S. Tyagi, M. L. Gennaro, and R. J. North. 2003. Expression of Th1-mediated immunity in mouse lungs induces a Mycobacterium tuberculosis transcription pattern characteristic of nonreplicating persistence. Proc. Natl. Acad. Sci. U.S.A 100:241-246.
20. Timm, J., F. A. Post, L. G. Bekker, G. B. Walther, H. C. Wainwright, R. Manganelli, W. T. Chan, L. Tsenova, B. Gold, I. Smith, G. Kaplan, and J. D. McKinney. 2003. Differential expression of iron-, carbon-, and oxygen-responsive mycobacterial genes in the lungs of chronically infected mice and tuberculosis patients. Proc. Natl. Acad. Sci. U.S.A 100:14321-14326.
21. Arend, S. M., P. Andersen, K. E. van Meijgaarden, R. L. Skjot, Y. W. Subronto, J. T. van Dissel, and T. H. Ottenhoff. 2000. Detection of active tuberculosis infection by T cell responses to early-secreted antigenic target 6-kDa protein and culture filtrate protein 10. J. Infect. Dis. 181:1850-1854.
22. Arend, S. M., A. Geluk, K. E. van Meijgaarden, J. T. van Dissel, M. Theisen, P. Andersen, and T. H. Ottenhoff. 2000. Antigenic equivalence of human T-cell responses to Mycobacterium tuberculosis-specific RD1-encoded protein antigens ESAT-6 and culture filtrate protein 10 and to mixtures of synthetic peptides. Infect. Immun. 68:3314-3321.
23. Franken, K. L., H. S. Hiemstra, K. E. van Meijgaarden, Y. Subronto, J. den Hartigh, T. H. Ottenhoff, and J. W. Drijfhout. 2000. Purification of his-tagged proteins by immobilized chelate affinity chromatography: the benefits from the use of organic solvent. Protein Expr. Purif. 18:95-99.
24. Ottenhoff, T. H., D. G. Elferink, J. Hermans, and R. R. de Vries. 1985. HLA class II restriction repertoire of antigen-specific T cells. I. The main restriction determinants for antigen presentation are associated with HLA-D/DR and not with DP and DQ. Hum. Immunol. 13:105-116.
25. Voskuil, M. I., K. C. Visconti, and G. K. Schoolnik. 2004. Mycobacterium tuberculosis gene expression during adaptation to stationary phase and low-oxygen dormancy. Tuberculosis. (Edinb.) 84:218-227.
26. Florczyk, M. A., L. A. McCue, A. Purkayastha, E. Currenti, M. J. Wolin, and K. A. McDonough. 2003. A family of acr-coregulated Mycobacterium tuberculosis genes shares a common DNA motif and requires Rv3133c (dosr or devr) for expression. Infect. Immun. 71:5332-5343.
27. Park, H. D., K. M. Guinn, M. I. Harrell, R. Liao, M. I. Voskuil, M. Tompa, G. K. Schoolnik, and D. R. Sherman. 2003. Rv3133c/dosR is a transcription factor that mediates the hypoxic response of Mycobacterium tuberculosis. Mol. Microbiol. 48:833-843.
28. Malhotra, V., D. Sharma, V. D. Ramanathan, H. Shakila, D. K. Saini, S. Chakravorty, T. K. Das, Q. Li, R. F. Silver, P. R. Narayanan, and J. S. Tyagi. 2004. Disruption of response regulator gene, devr, leads to attenuation in virulence of Mycobacterium tuberculosis. FEMS Microbiol. Lett. 231:237-245.
29. Yuan, Y., D. D. Crane, R. M. Simpson, Y. Q. Zhu, M. J. Hickey, D. R. Sherman, and C. E. Barry, III. 1998. The 16-kDa alpha-crystallin (Acr) protein of Mycobacterium tuberculosis is required for growth in macrophages. Proc. Natl. Acad. Sci. U.S.A 95:9578-9583.
30. Monahan, I. M., J. Betts, D. K. Banerjee, and P. D. Butcher. 2001. Differential expression of mycobacterial proteins following phagocytosis by macrophages. Microbiology 147:459-471.
31. Florczyk, M. A., L. A. McCue, R. F. Stack, C. R. Hauer, and K. A. McDonough. 2001. Identification and characterization of mycobacterial proteins differentially expressed under standing and shaking culture conditions, including Rv2623 from a novel class of putative ATP-binding proteins. Infect. Immun. 69:5777-5785.
32. Boon, C., R. Li, R. Qi, and T. Dick. 2001. Proteins of Mycobacterium bovis BCG induced in the Wayne dormancy model. J. Bacteriol. 183:2672-2676.
33. Boon, C. and T. Dick. 2002. Mycobacterium bovis BCG response regulator essential for hypoxic dormancy. J. Bacteriol. 184:6760-6767.
34. Ottenhoff, T. H., F. A. Verreck, E. G. Lichtenauer-Kaligis, M. A. Hoeve, O, Sanal, and J. T. van Dissel. 2002. Genetics, cytokines and human infectious disease: lessons from weakly pathogenic mycobacteria and salmonellae. Nat. Genet. 32:97-105.
35. Flynn, J. L., J. Chan, K. J. Triebold, D. K. Dalton, T. A. Stewart, and B. R. Bloom. 1993. An essential role for interferon gamma in resistance to Mycobacterium tuberculosis infection. J. Exp. Med. 178:2249-2254.
36. Mayuri, G. Bagchi, T. K. Das, and J. S. Tyagi. 2002. Molecular analysis of the dormancy response in Mycobacterium smegmatis: expression analysis of genes encoding the DevR-DevS two-component system, Rv3134c and chaperone alpha-crystallin homologues. FEMS Microbiol. Lett. 211:231-237.
37. Behr, M. A., M. A. Wilson, W. P. Gill, H. Salamon, G. K. Schoolnik, S. Rane, and P. M. Small. 1999. Comparative genomics of BCG vaccines by whole-genome DNA microarray. Science 284:1520-1523.
38. Colditz, G. A., T. F. Brewer, C. S. Berkey, M. E. Wilson, E. Burdick, H. V. Fineberg, and F. Mosteller. 1994. Efficacy of BCG vaccine in the prevention of tuberculosis. Meta-analysis of the published literature. JAMA 271:698-702.
39. Brandt, L., C. J. Feino, O. A. Weinreich, B. Chilima, P. Hirsch, R. Appelberg, and P. Andersen. 2002. Failure of the Mycobacterium bovis BCG vaccine: some species of environmental mycobacteria block multiplication of BCG and induction of protective immunity to tuberculosis. Infect. Immun. 70:672-678.
40. Vekemans, J., M. O. Ota, J. Sillah, K. Fielding, M. R. Alderson, Y. A. Skeiky, W. Dalemans, K. P. McAdam, C. Lienhardt, and A. Marchant. 2004. Immune responses to mycobacterial antigens in the Gambian population: implications for vaccines and immunodiagnostic test design. Infect. Immun. 72:381-388.
41. Ravn, P., H. Boesen, B. K. Pedersen, and P. Andersen. 1997. Human T cell responses induced by vaccination with Mycobacterium bovis bacillus Calmette-Guerin. J. Immunol. 158:1949-1955.
42. Lim, A., M. Eleuterio, B. Hutter, B. Murugasu-Oei, and T. Dick. 1999. Oxygen depletion-induced dormancy in Mycobacterium bovis BCG. J. Bacteriol. 181:2252-2256.
43. Parker, K. C., M. A. Bednarek, and J. E. Coligan. 1994. Scheme for ranking potential HLA-A2 binding peptides based on independent binding of individual peptide side-chains. J. Immunol. 152:163. HLA_BIND (http://bimas.cit.nih.gov/molbio/hla_bind/)
44. Rammensee, Friede, Stevanovic, MHC ligands and peptide motifs: 1st listing, Immunogenetics 41, 178-228, 1995; SYFPEITHI (http://www.syfpeithi.de/) and Rammensee, Bachmann, Stevanovic:MHC ligands and peptide motifs. Landes Bioscience 1997 (International distributor—except North America: Springer Verlag GmbH & Co. KG, Tiergartenstr. 17, D-69121 Heidelberg
45. Buus S, Lauemoller S L, Worning P, Kesmir C, Frimurer T, Corbet S, Fomsgaard A, Hilden J, Holm A, Brunak S. Sensitive quantitative predictions of peptide-MHC binding by a ‘Query by Committee’ artificial neural network approach, in Tissue Antigens., 62:378-84, 2003; NetMHC (http://www.cbs.dtu.dk/services/NetMHC/)
46. Nielsen M, Lundegaard C, Worning P, Lauemoller S L, Lamberth K, Buus S, Brunak S, Lund O., Reliable prediction of T-cell epitopes using neural networks with novel sequence representations. Protein Sci., 12:1007-17, 2003.
47. Improved prediction of MHC class I and class II epitopes using a novel Gibbs sampling approach, Nielsen M, Lundegaard C, Worning P, Hvid C S, Lamberth K, Buus S, Brunak S, Lund O., Bioinformatics, 20(9):1388-97, 2004.
48. Sturniolo, T. et al., Nature Biotechnology 17, 555-562, 1999, Generation of tissue-specific and promiscuous HLA ligand databases using DNA chips and virtual HLA class II matrices; TEPITOPE (http://www.vaccinome.com/pages/597444/).

1. A method for inducing an immune response against a Mycobacterium infection in a vertebrate, the method comprising the step of administering to the vertebrate a composition comprising a source of one or more polypeptides selected from the group consisting of M. tuberculosis dormancy (DosR) regulon sequences: Rv0079, Rv0569, Rv1733c, Rv1738, Rv1813c, Rv1996, Rv2007c, Rv2029c (PfkB), Rv2030c, Rv2031c (HspX), Rv2032, Rv2626c, Rv2627c, Rv2628, Rv3126c, Rv3129, Rv3130c, Rv3132c, Rv3133c (DosR), Rv0080, Rv1737c (NarX), Rv1735c and Rv1736c (NarK2), and analogues, homologues or fragments thereof.

2. The method of claim 1 wherein the polypeptide is selected from the group consisting of Rv1733c, Rv2029c (PfkB), Rv2627c, Rv2628, Rv0080, Rv1737c (NarK2), Rv1735c and Rv1736c (NarX).

3. The method of claim 2 wherein the polypeptide is selected from the group consisting of Rv1733c, Rv2029c and Rv2627c.

4. The method of claim 2 wherein the polypeptide is obtained from the TB dormancy regulon (DosR) sequences Rv1733c, Rv2029c (PfkB) and Rv0080.

5. A composition for immunization against Mycobacterium infections comprising a source of one or more polypeptides selected from the group consisting of TB dormancy (DosR) regulon sequences: Rv0079, Rv0569, Rv1733c, Rv1738, Rv1813c, Rv1996, Rv2007c, Rv2029c (PfkB), Rv2030c, Rv2031c (HspX), Rv2032, Rv2626c, Rv2627c, Rv2628, Rv3126c, Rv3129, Rv3130c, Rv3132c and Rv3133c (DosR), Rv0080, Rv1737c (NarX), Rv1735c and Rv1736c (NarK2), and analogues, homologues or fragments thereof, and optionally comprising an adjuvant.

6. The composition according to claim 5 wherein the polypeptide is selected from the group consisting of Rv1733c, Rv2029c (PfkB), Rv2627c, Rv2628, Rv0080, Rv1737c (NarK2), Rv1735c and Rv1736c (NarX).

7. The composition according to claim 6 wherein the polypeptide is selected from the group consisting of Rv1733c, Rv2029c and Rv2627c.

8. The composition according to claim 6 wherein the polypeptide is obtained from the TB dormancy regulon (DosR) sequences Rv1733c, Rv2029c (PfkB) and Rv0080.

9. The composition for immunization according to claim 5 wherein the adjuvant is selected from the group of adjuvants consisting of polyI:C, CpG, LPS, lipid A and derivatives thereof, IC31, QS21, lipopeptide Pam3Cys, bacterial flagellins, DDA/MPL, DDA/TDB and soluble LAG3.

10. The composition for immunization according to claim 5 wherein the Mycobacterium polypeptide is selected from Mycobacterium TB complex species M. tuberculosis, M. bovis, M. bovis BCG, M. africanum, M. canetti and M. microti.

11. The composition for immunization according to claim 5 wherein the source of the polypeptide is a recombinant DNA molecule encoding the polypeptide, optionally comprised in a vector and/or in a genome.

12. The composition for immunization according to claim 5 wherein the source of the polypeptide is in a recombinant Mycobacterium, preferably Mycobacterium bovis Bacillus Calmette-Guerin (BCG).

13. The composition for immunization according to claim 5 wherein the polypeptide fragments are synthetic peptides, preferably between 18 and 45 amino acids in length, optionally overlapping or ligated, optionally containing additional amino acids, immuno-stimulating moieties and or protective groups to enhance solubility and increase stability in vivo.

14. The composition for immunization according to claim 5, further comprising a CD40 binding molecule selected from an antibody or fragment thereof or a CD40 ligand or a variant thereof.

15. The composition for immunization according to claim 5, further comprising an agonistic anti-4-1BB antibody or a fragment thereof, capable of stimulating the 4-1BB receptor.

16. The composition for immunization according to claim 5 wherein the composition further comprises a Mycobacterium antigen that is not specific for the latency stage.

17. A method for diagnosing Mycobacterium infections in a subject comprising the steps of:
a) contacting a sample of isolated bodily fluid and/or isolated white blood cells of the subject with one or more polypeptides or fragments thereof selected from the group of proteins encoded by the TB dormancy regulon consisting of: Rv1733c, Rv2029c (PfkB), Rv2627c, Rv2628, Rv0080, Rv1737c (NarK2), Rv1735c and Rv1736c (NarX); and,
b) detecting a humoral immune response by binding of antibodies to said polypeptide(s) or fragments thereof being indicative of Mycobacterium infection and/or;
c) detecting a cellular immune response by specific proliferation and/or cytokine production and/or expression of extra- or intracellular activation markers.

18. The method according to claim 17 wherein the polypeptides are selected from the group consisting of Rv1733c, Rv2029c, Rv2627c and Rv0080.

19. A diagnostic kit for diagnosing Mycobacterium infections in a subject comprising one or more polypeptides of claim 1 and reagents to assay and quantify antibody binding to said polypeptides.
